(19) United States
(12) Patent Application Publication
(10) Pub. No.: US 2025/0154115 A1
(43) Pub. Date: May 15, 2025

(54) ARYLBENZOISOXAZOLE COMPOUNDS AS IP6K AND IPMK INHIBITORS AND METHODS OF USE THEREOF

(71) Applicant: THE UNIVERSITY OF NORTH CAROLINA AT CHAPEL HILL, Chapel Hill, NC (US)

(72) Inventors: Xiaodong Wang, Chapel Hill, NC (US); Yubai Zhou, Cary, NC (US)

(21) Appl. No.: 18/835,870

(22) PCT Filed: Feb. 10, 2023
(86) PCT No.: PCT/US23/12814

§ 371 Date: Aug. 5, 2024

Related U.S. Application Data
(60) Provisional application No. 63/309,042, filed on Feb. 11, 2022.

(51) Int. Cl. C07D 261/20; A61K 31/423; A61K 31/4245; A61K 31/4439; A61K 31/454; A61K 31/519; A61K 31/5377; C07D 413/04; C07D 413/10; C07D 413/12; C07D 413/14

(52) U.S. Cl. CPC C07D 261/20; A61K 31/423; A61K 31/4245; A61K 31/4439; A61K 31/454; A61K 31/519; A61K 31/5377; C07D 413/04; C07D 413/10; C07D 413/12; C07D 413/14

(57) ABSTRACT

Arylbenzoisoxazole compounds of Formula II, where R¹ and R² are defined herein, are useful for inhibiting isoforms of IP6K and IPMK, and for treating diseases and disorders such as non-alcoholic fatty liver disease (NAFLD), non-alcoholic steatohepatitis (NASH), cancer, and viral infections. Methods of using compounds of Formula II for prevention or treatment of such diseases and disorders, pharmaceutical compositions comprising the compounds, and methods of preparing the compounds are also disclosed.

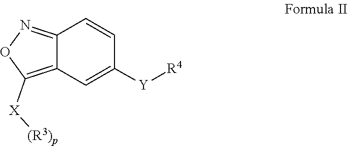

### TECHNICAL FIELD

The present disclosure relates to arylbenzoisoxazole containing compounds for treating diseases and disorders mediated by IP6K and IPMK inhibitors. The disclosure also relates to compositions comprising the compounds and methods of using the same to treat obesity and obesity-related diseases including various forms of non-alcoholic fatty liver disease (NAFLD) and non-alcoholic steatohepatitis (NASH), hyperphosphataemia, cancer, fungi infections, parasite infections, and viral infections.

### BACKGROUND

The incidence of worldwide obesity has more than doubled since 1980.¹-3 In obesity, excess fat accumulation causes adipocyte dysfunction, releases inflammatory cytokines, promotes insulin resistance, and reduces the fat-storing ability of adipocytes. Thus, obesity significantly increases the risk of co-morbidities such as type 2 diabetes mellitus (T2DM), hypertension, dyslipidemia, cardiovascular disease, nonalcoholic fatty liver disease/nonalcoholic steatohepatitis (NAFLD/NASH), reproductive dysfunction, respiratory abnormalities, psychiatric and neurodegenerative diseases, and certain types of cancer.⁴ Consequently, obesity greatly contributes to economic and social burdens.⁶ A combination of lifestyle modification and pharmacology has been shown to have beneficial effects in combating obesity.^{2, 7-8} Even a 5-10% loss in body weight/fat has been shown to decrease liver- and cardio-metabolic diseases in human patients.⁹⁻¹⁰ Yet, limited success has been achieved in reducing body weight for an extended period.¹⁻³

The inositol pyrophosphate (PP—IP) biosynthetic pathway has been identified as a target in metabolic diseases, osteoporosis, thromboembolism, infection, cancer metastasis and aging.¹¹⁻²² Inositol pyrophosphates are conserved eukaryotic messenger molecules with functionally significant and highly “energetic” diphosphate groups.²³⁻²⁴ They play crucial roles in diverse cellular functions, including insulin secretion and signaling,^{12, 25-26} ATP production,²⁷ DNA damage sensing,²⁸ and repair.²⁹ Many of these effects contribute to an over-arching role for PP-IPs in regulating bioenergetic homeostasis.^{23-24, 30} The initial and primary reaction in the PP—IP synthetic pathway is the phosphorylation of inositol hexakisphosphate (InsP₆) to 5-diphosphoinositol pentakisphosphate (5-InsP₇) by a family of small molecule kinases known as IP6K1, IP6K2 and IP6K3 (FIG. 1).

IP6K1 and IP6K2 are expressed in most tissues, while IP6K3 is mainly expressed in the heart, skeletal muscle, and brain.^{16, 20} IP6K1 regulates metabolism in vivo by several mechanisms.^{14, 20} This isoform promotes insulin secretion from pancreatic β cells,²⁵ but attenuates certain aspects of insulin signaling.¹² Moreover, IP6K1 reduces whole-body energy expenditure by inhibiting adipocyte thermogenesis.^{11, 15} Consequently, whole body and adipocyte-specific Ip6k1-KO mice display increased insulin sensitivity and energy expenditure and are protected from high fat diet (HFD)-induced obesity, hyperinsulinemia, and insulin resistance.^{11-12, 15} Furthermore, whole-body- or hepatocyte-specific Ip6k1 deletion ameliorates NAFLD and NASH in mice.²² Ip6k2 deletion in mice decreases cancer cell migration, invasion, and tumor metastasis.²¹ Deletion of Ip6k3 protects mice from age-induced fat accumulation and insulin resistance.¹⁶

These observations suggest that it would be useful to develop IP6K inhibitors to dissect the alternate biological functions of each isoform,³¹ and potentially to further develop drug candidates, particularly to target for obesity and obesity-induced metabolic and other diseases.¹⁴ Thus, the IP6Ks are potential targets for mediation of diseases and disorders associated with IP6K function; for example, inositol hexakisphosphate 5-kinase 1 (IP6K1) is a potential target for NAFLD/NASH treatment due to its pleiotropic effects on metabolic parameters (Zhou et al. J. Med. Chem. 2022, 65, 9,6869).

Recent publications also demonstrated the applications of IP6K inhibitors in hyperphosphataemia and IPMK inhibitors in fungi infection (Moritoh et al. Nature communications 2021, 12 (1), 1-15; Desmarini et al. Biomolecularesm 2022, 12(10), 1526).

The presently disclosed subject matter provides heterocyclic compounds having IP6K-and IPMK-inhibitory action useful as a prophylactic or therapeutic agents for the treatment of diseases such as NAFLD/NASH, hyperphosphataemia, fungi infection, glioblastoma, and coronavirus. These and other aspects are addressed by the disclosures herein.

### SUMMARY

In accordance with the purpose(s) of the currently disclosed subject matter, as embodied and broadly described herein, in one aspect relates to a compound of Formula Ia:

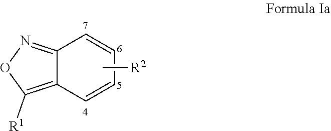

wherein R¹ is substituted or unsubstituted aryl or heteroaryl, and R² is halo, alkyl, alkenyl, carboxylic acid, ester, amide, sulfonamide, sulfonic acid, phosphonate ester, heteroaryl, or heterocycloalkyl.

In an embodiment, the compounds disclosed herein comprise a compound of Formula II:

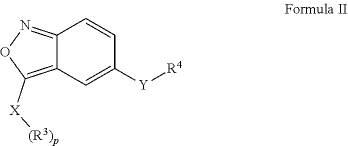

wherein X is aryl or heteroaryl;
R³ is, in each instance, selected from the group consisting of alkyl, cycloalkyl, heterocycloalkyl, aryl, heteroaryl, halo, -D, —OH, —OR⁵, —NH—COR⁶, and —CONR⁷R⁸;
p is an integer from 0 to 5;
Y is present or absent, and, when present, selected from the group consisting of alkyl, cycloalkyl, and alkenyl;
R⁴ is selected from the group consisting of —CO₂H, —CO₂R⁵—CONR⁷R⁸, —SO₂—NHR⁶, —SO₃H, —P(O)(OH)(OR⁹), heteroaryl, and heterocycloalkyl;
wherein R⁵ is selected from alkyl, aryl, heteroaryl, and heterocycloalkyl; R⁶ is selected from alkyl, cycloalkyl, aryl, heterocycloalkyl, and heteroaryl; R⁷ and R⁸ are each independently selected from hydrogen, alkyl, cycloalkyl, heterocycloalkyl, aryl, and heteroaryl; R⁹ is selected from alkyl, aryl, heteroaryl, and heterocycloalkyl; and R¹⁰ and R¹¹ are each independently selected from hydrogen, alkyl, cycloalkyl, heterocycloalkyl, aryl, and heteroaryl.

In another aspect, the subject matter described herein is directed to a pharmaceutical composition comprising a compound of Formula I in a pharmaceutically acceptable carrier or excipient.

In another aspect, the subject matter described herein is directed to a method of preventing or treating a disease or disorder mediated by IP6K and/or IPMK, the method comprising administering a therapeutically effective amount of a compound of Formula I to a subject in need thereof. In an embodiment, the disease or disorder is: NAFLD, NASH, hyperphosphataemia, fungi infection, glioblastoma, or coronavirus.

In another aspect, the subject matter described herein is directed to a method of preparing a compound of Formula I, the method comprising the steps of: 1) contacting a compound with the structure of Formula IV with a Wittig reagent, and 2) saponification of an ester group.

In another aspect, the subject matter described herein is directed to a kit for treating a disease or disorder mediated by IP6K and/or IPMK, the kit comprising: 1) a pharmaceutical composition comprising a compound of Formula I, and 2) instructions for use.

In another aspect, the subject matter described herein includes a compound of Formula I for use as a medicament, and for use in treating a disease or disorder mediated by IP6K and/or IPMK. In another aspect, the subject matter described herein includes the use of a compound of Formula I in the manufacture of a medicament for the treatment of a disease or disorder mediated by IP6K and/or IPMK. In an embodiment, the disease or disorder is: NAFLD, NASH, hyperphosphataemia, fungi infection, glioblastoma, or coronavirus.

These and other aspects are disclosed in further detail below.

### BRIEF DESCRIPTION OF THE FIGURES

FIG. 1 depicts a schematic of the application of an enzyme-coupled assay to determine the potency of TNP against IP6K1. FIG. 1 shows chair conformations of InsP₆ and 5-InsP₇ in a graphical representation of the coupled-enzyme assay; released Pi was assayed using Malachite Green.

FIGS. 2A-E show results from interactions of compound 20 with IP6Ks. FIGS. 2A-2C show dose/response relationships for inhibition by compound 20 of IP6K1, IP6K2 and IP6K3, respectively; data represent means and standard errors from either three or four independent experiments. FIG. 2D shows a thermogram (‘DP’=differential power) for ITC analysis of the interaction of compound 20 with IP6K2. FIG. 2E shows the corresponding Wiseman plot. The ITC data came from a representative experiment, typical of three.

FIGS. 3A-3E show results from the evaluation of the specificity and potency of compound 20. FIG. 3A shows a representative HPLC analysis of InsP₅, InsP₆, InsP₇ and InsP₈ in HCT116 cells after a 3 hour treatment with either vehicle control (black symbols) or 2.5 μM compound 20 (dark grey symbols). FIG. 3B shows mean and standard errors from three independent experiments, performed as in FIG. 3A. FIG. 3C shows the assay of InsP₇ kinase activity of PPIP5K2 in the presence of either 2.5 μM compound 20 (dark grey bar) or vehicle control (black bar). FIGS. 3D and 3E show dose/dependent effects of 3- and 18-hour treatments, respectively, of compound 20 upon [³³P]-Pi efflux from HCT116 cells. The broken lines indicate the 30% of total [³³P]-Pi efflux that is not regulated by InsP₈. Data in FIGS. 3C-3E represent means and standard errors from either three or four independent experiments.

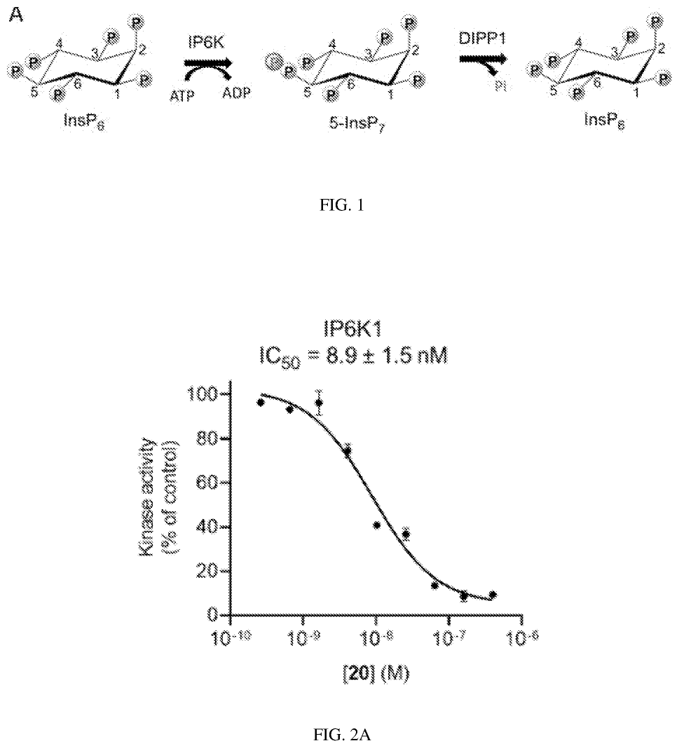

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

N2-(m-Trifluorobenzyl)-N6-(p-nitrobenzyl)purine (TNP) (compound 1) (Scheme 1 below) is the only widely used small molecule pan-IP6K inhibitor, which acts by competing with ATP binding.³² Treatment of diet-induced obese mice with TNP ameliorates obesity, insulin resistance and fatty liver, suggesting that targeting the IP6K pathway is pharmacologically tractable.³³ However, TNP is not a high-quality chemical probe because of its low potency, poor solubility and off-target effects.^{20, 33} In 2019, the Barrow lab reported modified TNP analogs with improved solubility, however, still with only moderate potency—for example, their most active compound (compound 2) exhibited an IC₅₀ value against IP6K1 of 0.75 μM; the IC₅₀ values against IP6K2 and IP6K3 were 20 and 15 μM respectively.³⁴

Others recently described screened compound libraries curated by NCATS and identified LI-2242 (compound 4) as an IP6K inhibitor (IC₅₀: IP6K1 31 nM; IP6K2 42 nM; IP6K3 8.7 nM).³⁸ LI-2242 is structurally very similar to the IP6K inhibitor SC-919 (compound 5), a compound that was initially disclosed by Takeda Pharmaceutical Company Ltd. in 2018.39 More recently, the activity of compound 5 in a chronic kidney disease mice model was described.⁴⁰

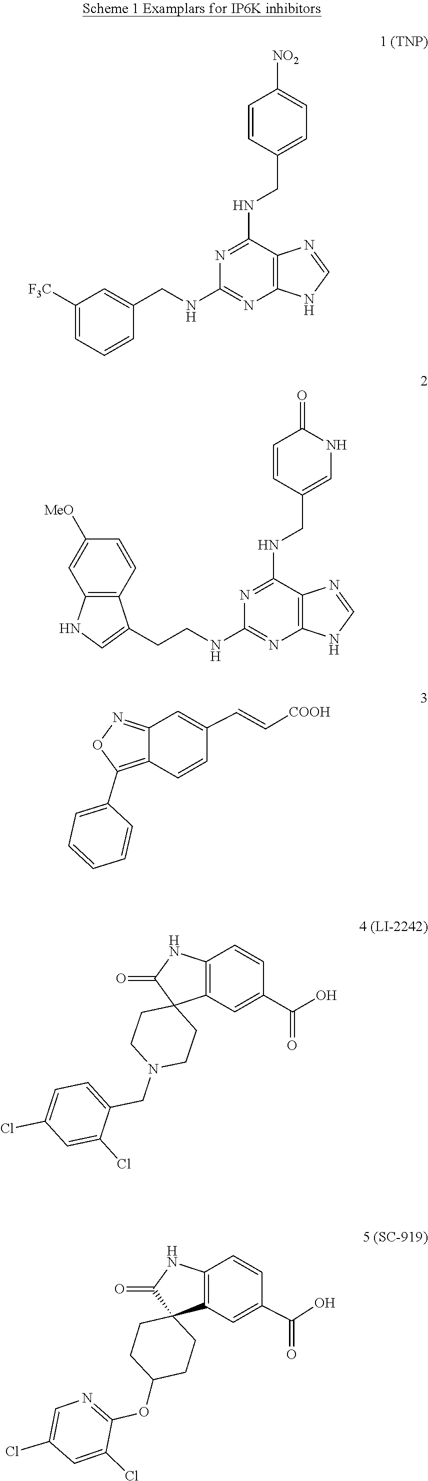

In contrast to methods previously used to identify potential IP6K inhibitors, an alternate approach was used to identify new lead compounds as IP6K inhibitors, based on these enzymes' ATP-binding sites being similar in nature to those of protein kinases, including the relative positions of the nucleotide-binding residues.³⁵ IP6Ks have unique structural elements in their nucleotide-binding sites that restrict ATP affinities to >10-fold lower values than all other inositol phosphate kinases and almost all protein kinases.^{32, 36} This permits inhibitor selectivity towards IP6Ks; therefore, a targeted screen against IP6K2 was performed using a library of protein kinase inhibitors.³⁷ Compound 3, which has a benzoisoxazole scaffold, was one of the hits identified in this screen with micromolar potency against IP6K2 (IC₅₀: 43.8 μM). Once compound 3 was identified, a ligand-based drug design approach was used in which various substituents were introduced into the benzoisoxazole-based compound 3.

The ligand-based drug design approach was necessitated by the fact that to date, none of the full-length human IP6Ks have yielded a crystal structure. Although the core structure of an IP6K ortholog from Entamoeba histolytica has been solved,⁴¹ that construct lacked the G-loop, which is a critical feature of the ATP-binding pocket.³⁵ Since this pharmacophore space within the IP6Ks is not defined, alternate options were required for exploring inhibitor scaffolds.

The presently disclosed subject matter provides heterocyclic compounds having IP6K and/or IPMK inhibitory action useful as a prophylactic or therapeutic agent for the treatment of diseases and disorders mediated by the IP6Ks, such as NAFLD/NASH and hyperphosphataemia, and by IPMK such as fungi infection, glioblastoma or coronavirus. In some embodiments, the heterocyclic compounds disclosed herein have a structure of Formula II, wherein X, Y, R³, R⁴, and p are defined as described herein.

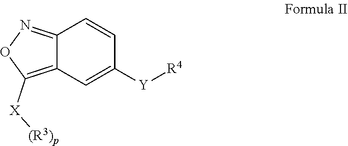

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to arrangement of steps or operational flow, plain meaning derived from grammatical organization or punctuation, or the number or type of aspects described in the specification.

### A. DEFINITIONS

Listed below are definitions of various terms used to describe this invention. These definitions apply to the terms as they are used throughout this specification, unless otherwise limited in specific instances, either individually or as part of a larger group.

As used in the specification and the appended claims, the singular forms “a,” “an” and “the” include plural referents unless the context clearly dictates otherwise. Thus, for example, reference to “an alkyl group” or “a phenyl” includes mixtures of two or more such alkyl groups or phenyls.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed. Further, unless specified by the term “integer,” the number specified includes fractions or numbers with decimals. For example, the range of “from about 1 to about 5” includes numbers such as 1, 1.1, 1.5, 2.0, 2.2, and so on. As used herein, the term “integer” refers to a number that is a whole number, and not a fraction.

References in the specification and concluding claims to parts by weight of a particular element or component in a composition denote the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compositions.

A weight percent (wt %) of a component, unless specifically stated to the contrary, is based on the total weight of the vehicle or composition in which the component is included.

As used herein, the terms “optional” and “optionally” mean that the subsequently described event or circumstance can or cannot occur, and that the description includes instances where said event or circumstance occurs and instances where it does not.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

As used herein, the terms “increase,” “increases,” “increased,” “increasing”, “improve,” “enhance,” and similar terms indicate an elevation in the specified parameter of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more.

As used herein, the terms “reduce,” “reduces,” “reduced,” “reduction,” “inhibit,” and similar terms refer to a decrease in the specified parameter of at least about 5%, 10%, 15%, 20%, 25%, 30%, 35%, 40%, 45%, 50%, 55%, 60%, 65%, 70%, 75%, 80%, 85%, 90%, 95%, 97%, or 100%.

As used herein, the “contacting” refers to reagents in close proximity so that a reaction may occur.

As used herein, “ambient temperature” or “room temperature” refers to a temperature in the range of about 20° C. to about 25° C.

As used herein, the term “alkyl” refers to a straight or branched chain hydrocarbon containing from 1 to 10 carbon atoms. Representative examples of alkyl include, but are not limited to: methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. These groups may be substituted with groups selected from halo (e.g., haloalkyl), alkyl, haloalkyl, alkenyl, alkynyl, cycloalkyl, cycloalkylalkyl, aryl, arylalkyl, heterocyclo, heterocycloalkyl, hydroxyl, alkoxy (thereby creating a polyalkoxy such as polyethylene glycol), alkenyloxy, alkynyloxy, haloalkoxy, cycloalkoxy, cycloalkylalkyloxy, aryloxy, arylalkyloxy, heterocyclooxy, heterocyclolalkyloxy, mercapto carboxy, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, ester, amide, nitro, or cyano.

The term “cycloalkyl” refers to a hydrocarbon 3-8 membered monocyclic or 7-14 membered bicyclic ring system having at least one saturated ring or having at least one non-aromatic ring, wherein the non-aromatic ring may have some degree of unsaturation. Cycloalkyl groups may be optionally substituted with one or more substituents. In one embodiment, 0, 1, 2, 3, or 4 atoms of each ring of a cycloalkyl group may be substituted by a substituent. Representative examples of cycloalkyl group include: cyclopropyl, cyclopentyl, cyclohexyl, cyclobutyl, cycloheptyl, cyclopentenyl, cyclopentadienyl, cyclohexenyl, cyclohexadienyl, and the like.

As used herein, the term “heterocycloalkyl” refers to a nonaromatic 3-8 membered monocyclic, 7-12 membered bicyclic, or 10-14 membered tricyclic ring system comprising 1-3 heteroatoms if monocyclic, 1-6 heteroatoms if bicyclic, or 1-9 heteroatoms if tricyclic, said heteroatoms selected from O, N, S, B, P or Si, wherein the nonaromatic ring system is completely saturated. Heterocycloalkyl groups may be optionally substituted with one or more substituents. In one embodiment, 0, 1, 2, 3, or 4 atoms of each ring of a heterocycloalkyl group may be substituted by a substituent. Representative heterocycloalkyl groups include piperidinyl, piperazinyl, tetrahydropyranyl, morpholinyl, thiomorpholinyl, 1,3-dioxolanyl, tetrahydrofuryl, tetrahydrothienyl, thienyl, and the like.

As used herein, the terms “alkenyl” and “alkene” refer to unsaturated aliphatic groups analogous in length and possible substitution to the alkyls described above, but that contain at least one double bond. For example, the term “alkenyl” includes straight-chain alkenyl groups (e.g., ethylenyl, propenyl, butenyl, pentenyl, hexenyl, heptenyl, octenyl, nonenyl or decenyl), branched-chain alkenyl groups and cycloalkenyl (alicyclic) groups (cyclopropenyl, cyclopentenyl, cyclohexenyl, cycloheptenyl or cyclooctenyl) groups. The term alkenyl further includes alkenyl groups that include oxygen, nitrogen, sulfur or phosphorous atoms replacing one or more carbons of the hydrocarbon backbone. In certain embodiments, a straight chain or branched chain alkenyl group with 10 or fewer carbon atoms in its backbone (e.g., C2-C10 for straight chain, C3-C10 for branched chain) is used. Likewise, cycloalkenyl groups may have from 3-8 carbon atoms in their ring structure, and more preferably have 5 or 6 carbons in the ring structure. The term C2-C10 includes alkenyl groups containing 2 to 10 carbon atoms. Certain alkene compounds of the invention may exist as a mixture of E and Z isomers, predominantly as E isomers, or predominantly Z isomers. In certain embodiments, compounds of the invention may be enriched in either the E or Z isomer. For example, a compound of the invention may have greater than 50%, 60%, 70%, 80%, 90%, or 95% or more of the E or Z isomer.

As used herein, the term “heteroaryl” or “heteroaromatic” refers to a monovalent aromatic radical of 5- or 6-membered rings, and includes fused ring systems (at least one of which is aromatic) of 5-20 atoms, containing one or more heteroatoms independently selected from nitrogen, oxygen, and sulfur. Examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl (including, for example, 3-amino-1,2-4-triazole or 3-mercapto-1,2,4-triazole), pyrazinyl (including, for example, aminopyrazine), tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, oxazol-2(3H)-onyl, and furopyridinyl. The heteroaryl groups are thus, in some embodiments, monocyclic or bicyclic. Heteroaryl groups are optionally substituted independently with one or more substituents described herein.

As used herein, the term “aryl” refers to a hydrocarbon monocyclic, bicyclic or tricyclic aromatic ring system. Aryl groups may be optionally substituted with one or more substituents. In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

As used herein, the term “substituted” refers to a moiety (such as an alkyl group), wherein the moiety is bonded to one or more additional organic radicals. In some embodiments, the substituted moiety comprises 1, 2, 3, 4, or 5 additional substituent groups or radicals. Suitable organic substituent radicals include, but are not limited to, hydroxyl, amino, mono-substituted amino, di-substituted amino, mercapto, alkylthiol, alkoxy, substituted alkoxy or haloalkoxy radicals, wherein the terms are defined herein. Unless otherwise indicated herein, the organic substituents can comprise from 1 to 4 or from 5 to 8 carbon atoms. When a substituted moiety is bonded thereon with more than one substituent radical, then the substituent radicals may be the same or different.

As used herein, the term “unsubstituted” refers to a moiety (such as an alkyl group) that is not bonded to one or more additional organic or inorganic substituent radical as described above, meaning that such a moiety is only substituted with hydrogens.

As used herein, the term “alkoxy”, used alone or as part of another group, means the radical —OR, where R is an alkyl group as defined herein.

As used herein, the terms “halo,” “halogen,” and “halide” refer to any suitable halogen, including —F, —Cl, —Br, and —I.

As used herein, the term “mercapto” refers to an —SH group.

As used herein, the term “cyano” refers to a —CN group.

As used herein, the term “carboxylic acid” refers to a —C(O)OH group.

As used herein, the term “hydroxyl” refers to an —OH group.

As used herein, the term “nitro” refers to an —NO₂ group.

As used herein, the term “sulfonyl” refers to the SO₂ group. The “sulfonyl” may refer to a sulfonyl group, which is, for example, an alkylsulfonyloxy group such as a methylsulfonyloxy or ethylsulfonyloxy group and an aromatic sulfonyloxy group such as a benzenesulfonyloxy or tosyloxy group.

As used herein, the terms “ether” and “alkylether” are represented by the formula Rₐ—O—R_{b}, where Rₐ and R_{b} can be, independently, an alkyl, cycloalkyl, alkenyl, cycloalkenyl, alkynyl, cycloalkynyl, aryl, or heteroaryl group as described herein. The term “polyether” as used herein is represented by the formula —(Rₐ—O—R_{b})ₓ—, where Rₐ and R_{b} can be, independently, an alkyl, cycloalkyl, alkenyl, cycloalkenyl, alkynyl, cycloalkynyl, aryl, or heteroaryl group described herein and “x” is from about 1 to about 500. Examples of polyether groups include polyethylene oxide, polypropylene oxide, and polybutylene oxide.

As used herein, the term “acyl”, used alone or as part of another group, refers to a —C(O)R radical, where R is any suitable substituent such as aryl, alkyl, alkenyl, alkynyl, cycloalkyl or other suitable substituent as described herein.

As used herein, the term “acylsilane,” used alone or as part of another group, refers to a —C(═O)—SiR₃— radical. R is any suitable substituent such as aryl, alkyl, alkenyl, alkynyl, cycloalkyl or other suitable substituent as described herein.

As used herein, the term “silyl” refers to a group of the formula —Si(R¹)(R²)(R³) where each of R¹, R², and R³ are independently hydrogen, alkyl, aryl, phenyl, phenyl-substituted alkyl, cycloalkyl or alkenyl; (R¹ R² R³ Si)ₙO, where R¹, R², and R³ are independently selected from the group comprising hydrogen and methyl; and R₃Si—X where X is a halide, including chlorine, bromine, and iodine, or p-toluenesulfonate.

As used herein, the term “silyl ether” refers to a silicon atom bonded to one or more carbon-containing groups via an oxygen atom (i.e., an ether linkage).

As used herein, the terms “alkylthio” and “thiyl,” used alone or as part of another group, refers to an alkyl group, as defined herein, appended to the parent molecular moiety through a thio moiety, as defined herein. Representative examples of alkylthio include, but are not limited, methylthio, ethylthio, tert-butylthio, hexylthio, and the like.

As used herein, the term “amino” means the radical —NH₂.

As used herein, the term “alkylamino” or “mono-substituted amino”, used alone or as part of another group, means the radical —NHR, where R is an alkyl group.

As used herein, the term “disubstituted amino”, used alone or as part of another group, means the radical —NRₐR_{b}, where Rₐ and R_{b} are independently selected from the groups alkyl, haloalkyl, alkenyl, alkynyl, cycloalkyl, cycloalkylalkyl, aryl, arylalkyl, heterocyclo, and heterocycloalkyl.

As used herein, the term “ester”, used alone or as part of another group, refers to a —C(O)OR radical, where R is any suitable substituent such as alkyl, cycloalkyl, alkenyl, alkynyl or aryl.

As used herein, the term “amide”, used alone or as part of another group, refers to a —C(O)NRₐR_{b} radical, where Rₐ and R_{b} are any suitable substituent such as alkyl, cycloalkyl, alkenyl, alkynyl or aryl.

It will be understood that the structures provided herein and any recitation of “substitution” or “substituted with” includes the implicit proviso that such structures and substitution are in accordance with permitted valence of the substituted atom and the substituent, and that the substitution results in a stable compound, e.g., which does not spontaneously undergo transformation such as by rearrangement, cyclization, elimination, etc.

As used herein, the term “stereoisomer” refers to compounds which have identical chemical constitution, but differ with regards to the arrangement of the atoms or groups in space. These “stereoisomers” have a “stereogenic center” which may be a chiral center.

As used herein, the term “chiral” refers to molecules which have the property of non-superimposability of the mirror image partner, while the term “achiral” refers to molecules which are superimposable on their mirror image partner.

As used herein, the term “diastereomers” refers to a stereoisomer with two or more centers of chirality and whose molecules are not mirror images of one another. Diastereomers have different physical properties, e.g., melting points, boiling points, spectral properties, and reactivity. Mixtures of diastereomers may separate under high resolution analytical procedures such as electrophoresis and chromatography.

As used herein, the term “enantiomers refers to two stereoisomers of a compound which are non-superimposable mirror images of one another. Stereochemical definitions and conventions used herein generally follow S. P. Parker, Ed., McGraw-Hill Dictionary of Chemical Terms (1984) McGraw-Hill Book Company, New York; and Eliel, E. and Wiley, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including, but not limited to, diastereomers, enantiomers and atropisomers, as well as mixtures thereof such as racemic mixtures, form part of the present invention.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder associated with a condition. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier, e.g., a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

Use of the word “inhibitor” herein is meant to mean a molecule that inhibits activity of an enzyme, such as IP6K. By “inhibit” herein is meant to decrease the activity of the target enzyme, as compared to the activity of that enzyme in the absence of the inhibitor. In some embodiments, the term “inhibit” means a decrease in IP6K activity of at least about 5%, at least about 10%, at least about 20%, at least about 25%, at least about 50%, at least about 60%, at least about 70%, at least about 80%, at least about 90%, or at least about 95%. In other embodiments, inhibit means a decrease in IP6K activity of about 5% to about 25%, about 25% to about 50%, about 50% to about 75%, or about 75% to 100%. In some embodiments, inhibit means a decrease in IP6K activity of about 95% to 100%, e.g., a decrease in activity of 95%, 96%, 97%, 98%, 99%, or 100%. Such decreases can be measured using a variety of techniques that would be recognizable by one of skill in the art, including in vitro kinase assays.

The term “administration” or “administering” includes routes of introducing the compound(s) to a subject to perform their intended function. Examples of routes of administration which can be used include injection (subcutaneous, intravenous, parenterally, intraperitoneally, intrathecal), topical, oral, inhalation, rectal and transdermal.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response. An effective amount is also one in which any toxic or detrimental effects (e.g., side effects) of the inhibitor compound are outweighed by the therapeutically beneficial effects.

The phrases “systemic administration,” “administered systemically”, “peripheral administration” and “administered peripherally” as used herein mean the administration of a compound(s), drug or other material, such that it enters the patient's system and, thus, is subject to metabolism and other like processes.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “subject” refers to animals such as mammals, including, but not limited to, primates (e.g., humans), cows, sheep, goats, horses, dogs, cats, rabbits, rats, mice and the like. In certain embodiments, the subject is a human.

### B. ARYLBENZOISOXAZOLE-CONTAINING COMPOUNDS

As described herein are compounds of Formula Ia. In some embodiments, the compounds of Formula I are inhibitors of IP6Ks and/or IPMK. As such, as embodied and broadly described herein, in one aspect relates to a compound of Formula Ia:

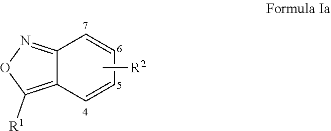

wherein R¹ is substituted or unsubstituted aryl or heteroaryl, and R² is halo, alkyl, alkenyl, carboxylic acid, ester, amide, sulfonamide, sulfonic acid, phosphonate ester, heteroaryl, or heterocycloalkyl.

In an embodiment, the compound of Formula Ia has one, two, three, or four R² groups, where in each instance, R² is selected from halo, alkyl, alkenyl, carboxylic acid, ester, amide, sulfonamide, sulfonic acid, phosphonate ester, heteroaryl, or heterocycloalkyl.

In a further embodiment, the compounds of Formula Ia comprise a compound of Formula Ib:

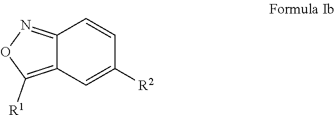

In a separate embodiment, the compounds comprise a compound of Formula Ic:

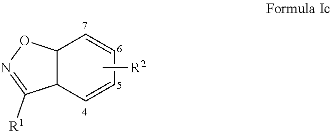

wherein R¹ and R² are as defined herein.

In an embodiment, the compounds disclosed herein comprise a compound of Formula II:

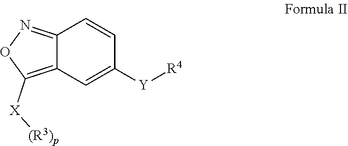

wherein X is aryl or heteroaryl;
R³ is, in each instance, selected from the group consisting of alkyl, cycloalkyl, heterocycloalkyl, aryl, heteroaryl, halo (e.g., —F, —Cl), -D, —OH, —OCF₃, —CF₃, —OR⁵, —NH—COR⁶, —CONR⁷R⁸, —SO₂—NHR⁹, and NR¹⁰R¹¹.
p is an integer from 0 to 5;
Y is present or absent, and when present selected from the group consisting of alkyl, cycloalkyl, and alkenyl;
R⁴ is selected from the group consisting of —CO₂H, —CO₂R⁵, —CONR⁷R⁸, —SO₂—NHR⁶, —SO₃H, —P(O)(OH)(OR⁹), heteroaryl, and heterocycloalkyl;
wherein R⁵ is selected from alkyl, aryl, heteroaryl, and heterocycloalkyl; R⁶ is selected from alkyl, cycloalkyl, aryl, and heteroaryl; R⁷ and R⁸ are each independently selected from hydrogen, alkyl, cycloalkyl, heterocycloalkyl, aryl, and heteroaryl; R⁹ is selected from alkyl, aryl, heteroaryl, and heterocycloalkyl; and R¹⁰ and R¹¹ are each independently selected from hydrogen, alkyl, cycloalkyl, heterocycloalkyl, aryl, and heteroaryl.

As in any embodiment above, a compound wherein Y is alkenyl. In further embodiments, Y is —C═C— (i.e., ethylene).

As in any embodiment above, a compound wherein Y is cycloalkyl. In further embodiments, Y is cyclopropyl.

As in any embodiment above, a compound wherein Y is ethylene or cyclopropyl and R⁴ is —CO₂H.

As in any embodiment above, a compound wherein Y is absent and R⁴ is selected from the group consisting of —CO₂H, —CO₂R⁶, —CONR⁷R⁸, —SO₂—NHR⁶, —SO₃H, —P(O)(OH)(OR⁹), heteroaryl, and heterocycloalkyl. In some embodiments, R⁴ is a heteroaryl or heterocycloalkyl. In some embodiments, R⁴ is selected from the group consisting of

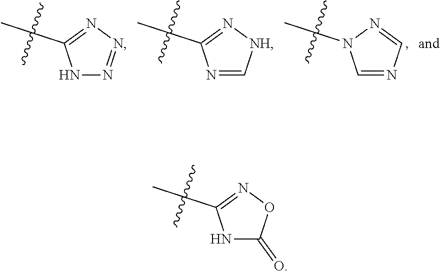 [SMILES: *c1nnn[nH]1 ; *c1nc[nH]n1 ; *n1cncn1 ; "," ; "and" ; *c1noc(=O)[nH]1]

In some embodiments, R⁴ is a tetrazole. In some embodiments, R⁴ is a 1,2,4-oxadiazol-5(4H)-one.

As in any embodiment above, R⁴ is —CONR⁷R⁸, wherein R⁷ and R⁸ are each independently selected from hydrogen, alkyl, cycloalkyl, heterocycloalkyl, aryl, and heteroaryl. In some embodiments, R⁷ is hydrogen and R⁸ is alkyl. In some embodiments, R⁷ is hydrogen and R⁸ is C1-C5 alkyl. In some embodiments, R⁷ is hydrogen and R⁸ is methyl, ethyl, or isopropyl. In some embodiments, R⁷ and R⁸ are both alkyl. In some embodiments, R⁷ and R⁸ are both methyl, ethyl, or isopropyl.

As in any embodiment above, R⁴ is —CO₂R⁵, wherein R⁵ is selected from alkyl, aryl, heteroaryl, and heterocycloalkyl. In some embodiments, R⁵ is alkyl. In some embodiments, R⁵ is methyl, ethyl, or isopropyl.

As in any embodiment above, a compound wherein Y is absent and R⁴ is —CO₂H.

As in any embodiment above, a compound wherein Y is absent and R⁴ is a tetrazole.

As in any embodiment above, a compound wherein Y is absent and R⁴ is a 1,2,4-oxadiazol-5(4H)-one.

As in any embodiment above, a compound wherein R⁵ is alkyl. In some embodiments, R⁵ is methyl.

As in any embodiment above, a compound wherein X is aryl. In some embodiments, X is phenyl. In some embodiments, X is unsubstituted phenyl.

As in any embodiment above, a compound wherein R³ is, in each instance, selected from the group consisting of alkyl, cycloalkyl, heterocycloalkyl, aryl, heteroaryl, halo, -D, —OH, —OR⁵, —NH—COR⁶, —CONR⁷R⁸, —SO₂—NHR⁹, and NR¹⁰R¹¹. In some embodiments, R³ is alkyl. In some embodiments, R³ is C1-C6 alkyl. In some embodiments, R³ is methyl. In some embodiments, R³ is alkyl substituted with halo, aryl, or heteroaryl. In some embodiments, R³ is —CF₃ or —CH₂— phenyl.

In some embodiments, R³ is a heteroaryl. In some embodiments, the heteroaryl is pyridyl or pyrimidyl. In some embodiments, the heteroaryl is substituted with at least one substituent (e.g., halo or hydroxyl), for example, —OCH₂(C₆H₅OCH₃). In some embodiments, R³ is a heterocycloalkyl. In some embodiments, R³ is piperidinyl, piperazinyl, morpholino, or pyrrolidinone. In some embodiments, R₃ is a beta-lactam moiety. In some embodiments, the heterocycloalkyl (e.g., piperidinyl) is substituted with cycloalkyl, heterocycloalkyl, —CH₂-(cycloalkyl), —CO-alkyl, —CO-cycloalkyl, or —CO-heterocycloalkyl. In some embodiments, the heterocycloalkyl (e.g., piperidinyl) is substituted with (C1-C6) cycloalkyl (e.g., cyclobutyl), heterocycloalkyl (e.g., trimethylene oxide moiety, beta-lactam moiety), —CH₂—((C1-C6)-cycloalkyl) (e.g., cyclopropyl), —CO—((C1-C6) alkyl) (e.g., -methyl, -isopropyl), —CO—((C1-C6) cycloalkyl) (e.g., butyl), or —CO-heterocycloalkyl. In some embodiments, the heterocycloalkyl is substituted with —CH₂CH₂CH₂— to afford a spirocyclic ligand, e.g.,

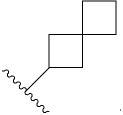 [SMILES: *C1CC2(CCC2)C1]

In some embodiments, the (C1-C6) cycloalkyl (e.g., cyclobutyl, cyclopentyl), —CH₂—((C1-C6)-cycloalkyl) (e.g., cycloalkyl is cyclopropyl), —CO—((C1-C6) alkyl) (e.g., alkyl is -methyl, -isopropyl), —CO—((C1-C6) cycloalkyl) (e.g., alkyl is butyl), or —CO-heterocycloalkyl are substituted with halo, alkyl, and/or ═O, for example,

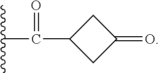 [SMILES: *C(=O)C1CC(=O)C1]

In some embodiments, R³ is aryl. In some embodiments, R³ is substituted with halo, alkyl, aryl, or heteroaryl. In some embodiments, R³ is phenyl. In some embodiments, R³ is phenyl substituted with at least one of methyl, —OCH₃, —OCF₃, —NH₂, —OC₆H₅, —C₆H₅, or halo (such as chloro).

In some embodiments, R³ is —OR⁵, wherein R⁵ is selected from alkyl, aryl, heteroaryl, and heterocycloalkyl. In some embodiments, R⁵ is aryl, such as phenyl. In some embodiments, R⁵ is heteroaryl, such as pyridyl. In some embodiments, the phenyl or pyridyl are substituted by one or two groups, such as a halogen or alkyl. In some embodiments, R⁵ is heterocycloalkyl, such as piperidinyl. In some embodiments, the heterocycloalkyl (e.g., piperidinyl) is substituted with an alkyl (e.g., methyl, ethyl, isopropyl), cycloalkyl (e.g., cyclopropyl, cyclobutyl, cyclopentyl), hetercycloalkyl, —CH₂-cycloalkyl (e.g., cycloalkyl is cyclopropyl, cyclobutyl, cyclopentyl), —CO— alkyl (e.g., alkyl is methyl, ethyl, isopropyl), —CO-cycloalkyl (e.g., alkyl is cyclopropyl, cyclobuty, cyclopentyl), —CO-heterocycloalkyl, or —CH₂CH₂OH. In some embodiments, the alkyl, cycloalkyl, hetercycloalkyl, —CH₂-cycloalkyl, —CO-alkyl, —CO-cycloalkyl, or —CO-heterocycloalkyl group can be further substituted. Exemplary substituents include alkyl, halogen, cycloalkyl, or —COOC(CH₃)₃ substituent(s).

In some embodiments, R⁵ is alkyl. In some embodiments, R⁵ is C1-C6 alkyl. In some embodiments, R⁵ is methyl or ethyl. In some embodiments, R⁵ is alkyl substituted with halo, aryl, heteroaryl, amino, cycloalkyl, or heterocycloalkyl. In some embodiments, R⁵ is —CF₃, —CH₂— phenyl, —CH₂—CH₂—N(CH₃)₂, —CH₂-heterocycloalkyl, or —CH₂—CH₂-heterocycloalkyl. In some embodiments, —CH₂—CH₂-heterocycloalkyl is —CH₂—CH₂-morpholino. In some embodiments, R⁵ is heterocycloalkyl, which may be further substituted. In some embodiments, R⁵ is a substituted or unsubstituted piperidinyl. In some embodiments, R⁵ is heterocycloalkyl substituted with alkyl, cycloalkyl, heteroarlkyl, —CO-cycloalkyl, CO-alkyl, —CH₂-cycloalkyl, or CH₂CH₂OH.

In some embodiments, R³ is —NH—COR⁶, wherein R⁶ is selected from alkyl, cycloalkyl, aryl, heterocycloalkyl, and heteroaryl. In some embodiments, R⁶ is an alkyl which is C1-C6 alkyl. In some embodiments, the C1-C6 alkyl is methyl, ethyl, or isopropyl. In some embodiments, the alkyl is substituted with a heterocycloalkyl such as morpholino, tetrahydropyranyl, or cyclopropyl. In some embodiments, the alkyl is —CH₂—CH₂-morpholino, —CH₂—CH₂-tetrahydropyran, or —CH₂—CH₂-cyclopropyl. In some embodiments, R⁶ is a cycloalkyl selected from cyclopropyl, cyclobutyl, cyclopentyl, and cyclohexyl. In some embodiments, R⁶ is an aryl which is a phenyl. In some embodiments, the phenyl is substituted with alkyl or halo (such as chloro). In some embodiments, R⁶ is a heteroaryl selected from pyridyl, pyrimidyl, and triazolyl. In some embodiments, R⁶ is heterocycloalkyl which is selected from piperidinone, piperidinyl, morpholino, azetidinyl, and pyrrolidinyl, any one of which may be substituted, e.g., with an alkyl substituent (i.e., methyl). In some embodiments, R6 is a heteroaryl (e.g., pyridyl) substituted with —OCH₂(C₆H₅)OCH₃ or —OH.

In some embodiments, R³ is —CONR⁷R⁸, wherein R⁷ and R⁸ are each independently selected from hydrogen, alkyl, cycloalkyl, heterocycloalkyl, aryl, and heteroaryl. In some embodiments, R⁷ is hydrogen and R⁸ is alkyl. In some embodiments, R⁷ is hydrogen and R⁸ is C1-C5 alkyl. In some embodiments, R⁷ is hydrogen and R¹ is methyl, ethyl, or isopropyl. In some embodiments, R⁷ is hydrogen and R⁸ is heterocycloalkyl. In some embodiments, R⁸ is heterocycloalkyl which is selected from piperidinone, piperidinyl, morpholino, azetidinyl, and pyrrolidinyl, any one of which may be substituted.

In some embodiments, R³ is —SO₂—NHR⁹, wherein R⁹ is selected from alkyl, aryl, heteroaryl, and heterocycloalkyl. In some embodiments, R⁹ is aryl, such as phenyl. In some embodiments, R⁹ is heteroaryl, such as pyridyl. In some embodiments, the phenyl or pyridyl are substituted by one or two groups, such as a halogen or alkyl.

In some embodiments, R⁹ is alkyl. In some embodiments, R⁹ is C1-C6 alkyl. In some embodiments, R⁹ is methyl or ethyl. In some embodiments, R⁹ is alkyl substituted with halo, aryl, heteroaryl, amino, or heterocycloalkyl. In some embodiments, R⁹ is —CF₃, —CH₂-phenyl, —CH₂—CH₂—N(CH₃)₂, or —CH₂—CH₂-heterocycloalkyl. In some embodiments, —CH₂—CH₂-heterocycloalkyl is —CH₂—CH₂-morpholino. In some embodiments, R⁹ is heterocycloalkyl, which may be further substituted. In some embodiments, R⁹ is a substituted or unsubstituted piperidinyl.

In some embodiments, R³ is —NR¹⁰R¹¹, wherein R¹⁰ and R¹¹ are each independently selected from hydrogen, alkyl, cycloalkyl, heterocycloalkyl, aryl, and heteroaryl. In some embodiments, R¹⁰ is hydrogen and R¹¹ is alkyl. In some embodiments, R¹⁰ is hydrogen and R¹¹ is C1-C5 alkyl. In some embodiments, R¹⁰ is hydrogen and R¹¹ is methyl, ethyl, or isopropyl. In some embodiments, R¹⁰ is hydrogen and R¹¹ is heterocycloalkyl. In some embodiments, R¹⁰ is heterocycloalkyl which is selected from piperidinone, piperidinyl, morpholino, azetidinyl, and pyrrolidinyl, any one of which may be substituted.

As in any embodiment above, a compound wherein the alkyl, alkenyl, cycloalkyl, heterocycloalkyl, aryl, and heteroaryl are optionally substituted with one or more groups independently selected from alkyl, hydroxyl, cyano, F, Cl, Br, —CH₃, —CH₂CH₃, —CH(CH₃)₂, —CH₂CH(CH₃)₂, —CH₂NH₂, —CH₂NHCH₃, —CH₂N(CH₃)₂, —CH₂CH₂NH₂, —CH₂CH₂CH₂NH₂, —CH₂CH₂CH₂CH₂NH₂, —CH₂CH(CH₃)NH₂, —CH₂CONH₂, —CH₂OH, —CH₂CH₂OH, —C(CH₃)₂OH, —CH(OH)CH(CH₃)₂, —C(CH₃)₂CH₂OH, —CH₂C(CH₃)₂OH, —CH₂CH₂SO₂CH₃, —CN, —CF₃, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NO₂, —NH₂, —NHCH₃, —N(CH₃)₂, —NHCOCH₃, —N(CH₃)COCH₃, —NHS(O)₂CH₃, —NHCH₂CH₂NH₂, —NHCH₂CH₂CH₂NH₂, —NHCH₂CH₂CH₂CH₂NH₂, —N(CH₃)C(CH₃)₂CONH₂, —N(CH₃)CH₂CH₂S(O)₂CH₃, —O, —OH, —OCH₃, —OCH₂CH₂OCH₃, —OCH₂CH₂NH₂, —OCH₂[(C₆H₄)OCH₃], —S(O)₂N(CH₃)₂, —SCH₃, —CH₂OCH₃, —S(O)₂CH₃, cyclopropyl, cyclobutyl, cyclopentyl, cyclohexyl, cycloheptyl, azetidinyl, azepanyl, oxetanyl, pyrrolidinyl, piperazinyl, piperidinyl, (piperidin-4-yl)ethyl, pyranyl, (piperidin-4-ylmethyl), morpholinomethyl, and morpholino.

As in any embodiment above, a compound wherein the —CO-alkyl, —CO-cycloalkyl, or —CO-heterocycloalkyl are optionally substituted with one or more groups independently selected from alkyl, hydroxyl, cyano, F, Cl, Br, —CH₃, —CH₂CH₃, —CH(CH₃)₂, —CH₂CH(CH₃)₂, —CH₂CH₂CH₂— —CH₂NH₂, —CH₂NHCH₃, —CH₂N(CH₃)₂, —CH₂CH₂NH₂, —CH₂CH₂CH₂NH₂, —CH₂CH₂CH₂CH₂NH₂, —CH₂CH(CH₃)NH₂, —CH₂CONH₂, —CH₂OH, —CH₂CH₂OH, —C(CH₃)₂OH, —CH(OH)CH(CH₃)₂, —C(CH₃)₂CH₂OH, —CH₂C(CH₃)₂OH, —CH₂CH₂SO₂CH₃, —CN, —CF₃, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NO₂, —NH₂, —NHCH₃, —N(CH₃)₂, —NHCOCH₃, —N(CH₃)COCH₃, —COOC(CH₃)₃, —NHS(O)₂CH₃, —NHCH₂CH₂NH₂, —NHCH₂CH₂CH₂NH₂, —NHCH₂CH₂CH₂CH₂NH₂, —N(CH₃)C(CH₃)₂CONH₂, —N(CH₃)CH₂CH₂S(O)₂CH₃, ═O, —OH, —OCH₃, —OCH₂CH₂OCH₃, —OCH₂CH₂NH₂, —OCH₂[(C₆H₄)OCH₃], —S(O)₂N(CH₃)₂, —SCH₃, —CH₂OCH₃, —S(O)₂CH₃, cyclopropyl, cyclobutyl, cyclopentyl, cyclohexyl, cycloheptyl, azetidinyl, azepanyl, oxetanyl, pyrrolidinyl, piperazinyl, piperidinyl, (piperidin-4-yl)ethyl, pyranyl, (piperidin-4-ylmethyl), morpholinomethyl, and morpholino.

In some embodiments, the compounds disclosed herein comprise a compound of Formula IIIa:

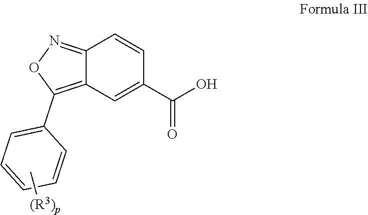

wherein R³ is, in each instance, selected from the group consisting of alkyl, cycloalkyl, heterocycloalkyl, aryl, heteroaryl, halo, —OH, —OR⁵, —NH—COR⁶, —CONR⁷R⁸—SO₂—NHR⁹, and NR¹⁰R¹¹; wherein R⁶ is selected from alkyl, cycloalkyl, aryl, heterocycloalkyl, and heteroaryl; R⁷ and R⁸ are each independently selected from hydrogen, alkyl, cycloalkyl, heterocycloalkyl, aryl, and heteroaryl; and p is an integer from 0 to 5.

As in any embodiment above, a compound wherein p is 1, 2, 3, 4, or 5. In some embodiments, p is 1.

As in any embodiment above, a compound where R³ is phenyl. In some embodiments, R³ is 4-phenyl.

In some embodiments, p is 1 and the compounds have the structure of Formulae IIIa, IIIb, or IIIc.

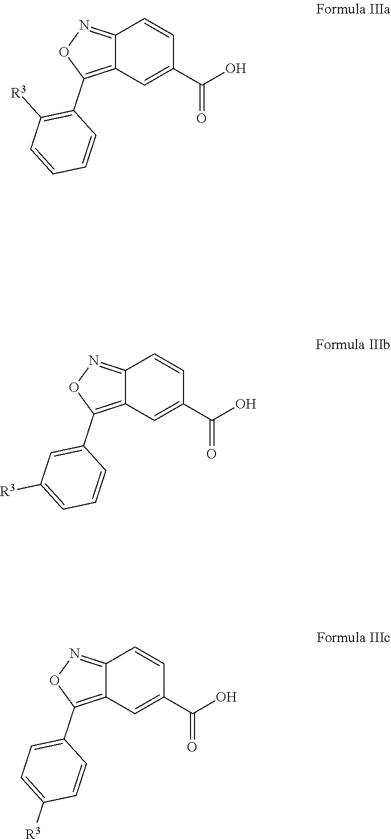

In some embodiments, the compound is a compound of Formula IIIc wherein R³ is phenyl. In a further embodiment, R³ is unsubstituted phenyl.

In some embodiments, when the compounds of Formula I have R²=COOH, R¹ is not phenyl, 4-methylphenyl, 4-fluorophenyl, 4-bromophenyl, 4-methoxyphenyl, or 2-methoxyphenyl. In an embodiment, when the compound of Formula I has R²=CONHCH₃, R¹ is not phenyl. In an embodiment, when the compound of Formula I has R²=—CH═CHCOOH, R¹ is not phenyl.

In some embodiments, the compounds disclosed herein comprise a compound of Formula IV or Formula V:

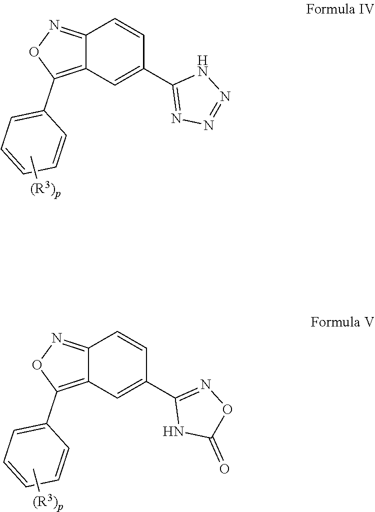

In some embodiments, the compounds have one of the following structures:

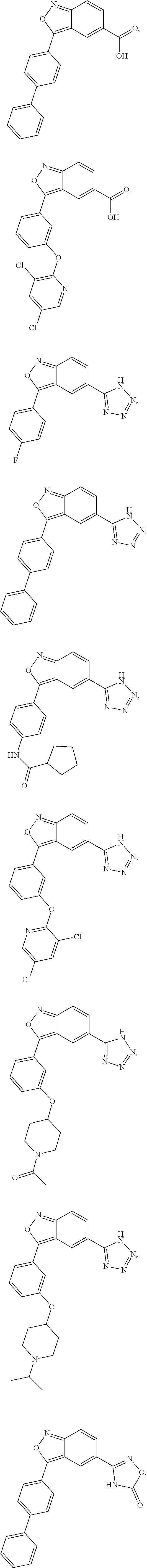

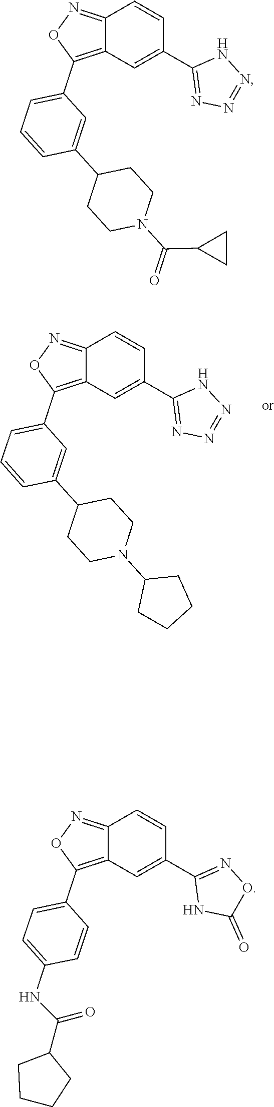 [SMILES: O=C(C1CC1)N1CCC(c2cccc(-c3onc4ccc(-c5nnn[nH]5)cc34)c2)CC1 ; c1cc(-c2onc3ccc(-c4nnn[nH]4)cc23)cc(C2CCN(C3CCCC3)CC2)c1 ; O=C(Nc1ccc(-c2onc3ccc(-c4noc(=O)[nH]4)cc23)cc1)C1CCCC1]

A compound from any one of Formulae I-V may be selected from the compounds listed in the tables disclosed herein. Compounds of Formulae I-V that are not listed in the disclosed tables are also within the scope of Formulae I-V.

The compounds disclosed herein may be in the form of a salt. Non-limiting examples thereof include metal salts, an ammonium salt, salts with organic base, salts with inorganic acid, salts with organic acid, salts with basic or acidic amino acid, and the like. Preferable examples of the metal salt include alkali metal salts such as sodium salt, potassium salt and the like; alkaline earth metal salts such as calcium salt, magnesium salt, barium salt and the like; an aluminum salt, and the like. Preferable examples of the salt with organic base include salts with trimethylamine, triethylamine, pyridine, picoline, 2,6-lutidine, ethanolamine, diethanolamine, triethanolamine, cyclohexylamine, dicyclohexylamine, tris(hydroxymethyl)aminomethane, N,N′-dibenzylethylenediamine and the like. Preferable examples of the salt with inorganic acid include salts with hydrochloric acid, hydrobromic acid, nitric acid, sulfuric acid, phosphoric acid and the like. Preferable examples of the salt with organic acid include salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like. Preferable examples of the salt with basic amino acid include salts with arginine, lysine, ornithine and the like. Preferable examples of the salt with acidic amino acid include salts with aspartic acid, glutamic acid and the like.

Among the salts, a pharmaceutically acceptable salt is preferable. For example, when a compound has an acidic functional group, examples thereof include inorganic salts such as alkali metal salts (e.g., sodium salt, potassium salt etc.), alkaline earth metal salts (e.g., calcium salt, magnesium salt etc.) and the like, ammonium salt etc., and when a compound has a basic functional group, examples thereof include salts with inorganic acid such as hydrochloric acid, hydrobromic acid, nitric acid, sulfuric acid, phosphoric acid and the like, and salts with organic acid such as acetic acid, phthalic acid, fumaric acid, oxalic acid, tartaric acid, maleic acid, citric acid, succinic acid, methanesulfonic acid, benzenesulfonic acid, p-toluenesulfonic acid and the like.

In an embodiment, the compound of Formula I is in salt form. In a further embodiment, the salt is a hydrochloride salt.

In some embodiments, the compounds disclosed herein are selective inhibitors. As used herein, the term “selective inhibitor” refers to compounds which display an increased selectivity for inhibiting a particular IP6K (such as IP6K1) when compared to inhibiting other IP6Ks or IPMK. In some embodiments, the compounds disclosed herein are pan-inhibitors. As used herein, the term “pan-inhibitor” refers to compounds that inhibit two or more IP6Ks in a similar potency range.

### C. PHARMACEUTICAL COMPOSITIONS

The presently disclosed compounds can be formulated into pharmaceutical compositions along with a pharmaceutically acceptable carrier. Compounds as disclosed herein can be formulated in accordance with standard pharmaceutical practice as a pharmaceutical composition. According to this aspect, there is provided a pharmaceutical composition comprising a compound as disclosed herein in association with a pharmaceutically acceptable diluent, carrier, or excipient.

A typical formulation is prepared by mixing a compound as disclosed herein and a carrier, diluent, or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound as disclosed herein or pharmaceutical composition thereof) or aid in the manufacturing of the pharmaceutical product (i.e., medicament).

The formulations may be prepared using conventional dissolution and mixing procedures. For example, the bulk drug substance (i.e., compound as disclosed herein or stabilized form of the compound), such as a complex with a cyclodextrin derivative or other known complexation agent, is dissolved in a suitable solvent in the presence of one or more of the excipients described above. The compound is typically formulated into pharmaceutical dosage forms to provide an easily controllable dosage of the drug and to enable patient compliance with the prescribed regimen.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical formulations may be prepared for various routes and types of administration. For example, a compound as disclosed herein having the desired degree of purity may optionally be mixed with pharmaceutically acceptable diluents, carriers, excipients or stabilizers (Remington's Pharmaceutical Sciences (1980) 16th edition, Osol, A. Ed.), in the form of a lyophilized formulation, milled powder, or an aqueous solution. Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment.

The compounds can be sterile. In particular, formulations to be used for in vivo administration should be sterile. Such sterilization is readily accomplished by filtration through sterile filtration membranes. The compound ordinarily can be stored as a solid composition, a lyophilized formulation or as an aqueous solution.

The pharmaceutical compositions comprising a compound as disclosed herein can be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the compound to be administered will be governed by such considerations, and is the minimum amount necessary to prevent, ameliorate, or treat the coagulation factor mediated disorder. Such amount is preferably below the amount that is toxic to the host or renders the host significantly more susceptible to bleeding.

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations of compounds may be prepared. Suitable examples of sustained-release preparations include semipermeable matrices of solid hydrophobic polymers containing a compound as disclosed herein, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate) and poly-D-(−)-3-hydroxybutyric acid.

The formulations include those suitable for the administration routes detailed herein. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of a compound as disclosed herein suitable for oral administration may be prepared as discrete units such as pills, capsules, cachets or tablets each containing a predetermined amount of a compound.

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Tablets, troches, lozenges, aqueous or oil suspensions, dispersible powders or granules, emulsions, hard or soft capsules, e.g., gelatin capsules, syrups or elixirs may be prepared for oral use. Formulations of compounds as disclosed herein intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

For treatment of the eye or other external tissues, e.g., mouth and skin, the formulations may be applied as a topical ointment or cream containing the active ingredient(s) in an amount of, for example, 0.075 to 20% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include a polyhydric alcohol, i.e., an alcohol having two or more hydroxyl groups such as propylene glycol, butane 1,3-diol, mannitol, sorbitol, glycerol and polyethylene glycol (including PEG 400), and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of the emulsions may be constituted from known ingredients in a known manner. While the phase may comprise solely an emulsifier, it may also comprise a mixture of at least one emulsifier and a fat or oil, or both a fat and an oil. A hydrophilic emulsifier included together with a lipophilic emulsifier may act as a stabilizer. Together, the emulsifier(s) with or without stabilizer(s) make up the so-called emulsifying wax, and the wax together with the oil and fat make up the so-called emulsifying ointment base which forms the oily dispersed phase of the cream formulations. Emulsifiers and emulsion stabilizers suitable for use in the formulation include TWEEN™ 60, Span® 80, cetostearyl alcohol, benzyl alcohol, myristyl alcohol, glyceryl mono-stearate and sodium lauryl sulfate.

Aqueous suspensions of compounds contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxybenzoate, one or more coloring agents, one or more flavoring agents and one or more sweetening agents, such as sucrose or saccharin.

The pharmaceutical compositions of compounds may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such 1,3-butanediol. The sterile injectable preparation may also be prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 mg to 1000 mg of active material compounded with an appropriate and convenient amount of carrier material which may vary from about 5% to about 95% of the total compositions (weight:weight). The pharmaceutical composition can be prepared to provide easily measurable amounts for administration. For example, an aqueous solution intended for intravenous infusion may contain from about 1 to 500 g of the active ingredient per milliliter of solution in order that infusion of a suitable volume at a rate of about 10 mL/hr to about 50 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of about 0.5 to 20% w/w, for example about 0.5 to 10% w/w, for example about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis disorders as described below.

Formulations suitable for vaginal administration may be presented as pessaries, tampons, creams, gels, pastes, foams or spray formulations containing in addition to the active ingredient such carriers as are known in the art to be appropriate.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The subject matter further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

In particular embodiments, the pharmaceutical composition comprising the presently disclosed compounds further comprise a chemotherapeutic agent. In some of these embodiments, the chemotherapeutic agent is an immunotherapeutic agent.

Administration of the compounds of the present invention can be effected by any method that enables delivery of the compounds to the site of action. These methods include oral routes, intraduodenal routes, parenteral injection (including intravenous, subcutaneous, intramuscular, intravascular or infusion), topical, inhalation and rectal administration.

The amount of the active compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

A dose may be administered once a day (QID), twice per day (BID), or more frequently, depending on the pharmacokinetic and pharmacodynamics properties, including absorption, distribution, metabolism, and excretion of the particular compound. In addition, toxicity factors may influence the dosage and administration regimen. When administered orally, the pill, capsule, or tablet may be ingested daily or less frequently for a specified period of time. The regimen may be repeated for a number of cycles of therapy.

The active compound may be applied as a sole therapy or in combination with one or more therapeutic agents. Such treatment may be achieved by way of the simultaneous, sequential or separate dosing of the individual components of treatment.

### D. ARTICLES OF MANUFACTURE

In another embodiment of the presently disclosed subject matter is an article of manufacture, or “kit,” containing materials useful for the treatment of the diseases and disorders described herein. The kit comprises a container comprising a compound of Formula I. The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister pack, etc. The container may be formed from a variety of materials such as glass or plastic. The container may hold a compound of Formula I or a formulation thereof which is effective for treating the condition and may have a sterile access port (for example, the container may be an intravenous solution bag or a vial having a stopper able to be pierced by a hypodermic injection needle). At least one active agent in the composition is a compound of Formula I. The label or package insert indicates that the composition is used for treating the condition of choice, such as cancer. In addition, the label or package insert may indicate that the patient to be treated is one having a disorder such as NAFLD or NASH. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the compound of Formula I and, if present, the second pharmaceutical formulation. For example, if the kit comprises a first composition comprising a compound of Formula I, and a second pharmaceutical formulation, the kit may further comprise directions for the simultaneous, sequential or separate administration of the first and second pharmaceutical compositions to a patient in need thereof.

In another embodiment, the kits are suitable for the delivery of solid oral forms of a compound of Formula I, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to one embodiment, a kit may comprise (a) a first container with a compound of Formula I contained therein; and optionally (b) a second container with a second pharmaceutical formulation contained therein, wherein the second pharmaceutical formulation comprises a second compound. Alternatively, or additionally, the kit may further comprise a third container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In certain other embodiments wherein the kit comprises a composition of Formula I and a second therapeutic agent, the kit may comprise a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), arc administered at different dosage intervals, or when titration of the individual components of the combination is desired by the prescribing physician.

### E. METHODS OF PREPARING

Compounds disclosed herein may be synthesized by synthetic routes that include processes analogous to those well-known in the chemical arts, particularly in light of the description contained herein, and those for other heterocycles described in: Comprehensive Heterocyclic Chemistry II, Editors Katritzky and Rees, Elsevier, 1997, e.g., Volume 3; Liebigs Annalen der Chemie, (9):1910-16, (1985); Helvetica Chimica Acta, 41:1052-60, (1958); Arzneimittel-Forschung, 40(12):1328-31, (1990), each of which are expressly incorporated by reference. Starting materials are generally available from commercial sources or are readily prepared using method well known to those skilled in the art (e.g., prepared by methods generally described in Louis F. Fieser and Mary Fieser, Reagents/or Organic Synthesis, v. 1-23, Wiley, N.Y. (1967-2006 ed.), or Beilsteins Handbuch der organischen Chemie, 4, Aufl. ed. Springer-Verlag, Berlin, including supplements (also available via the Beilstein online database)).

Synthetic chemistry transformations and protecting group methodologies (protection and deprotection) useful in synthesizing Formula I compounds and necessary reagents and intermediates are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); and L. Paquette, ed., Encyclopedia of Reagents for Organic Synthesis, John Wiley and Sons (1995) and subsequent editions thereof.

The General Procedures and Examples provide exemplary methods for preparing Formula I compounds. Those skilled in the art will appreciate that other synthetic routes may be used to synthesize the compounds. Although specific starting materials and reagents are depicted and discussed in the Figures, General Procedures, and Examples, other starting materials and reagents can be easily substituted to provide a variety of derivatives and/or reaction conditions. In addition, many of the exemplary compounds prepared by the described methods can be further modified in light of this disclosure using conventional chemistry well known to those skilled in the art.

In preparing compounds, protection of remote functionality (e.g., primary or secondary amine) of intermediates may be necessary. The need for such protection will vary depending on the nature of the remote functionality and the conditions of the preparation methods. Suitable amino-protecting groups include acetyl, trifluoroacetyl, t-butoxycarbonyl (BOC), benzyloxycarbonyl (CBz) and 9-fluorenylmethyleneoxycarbonyl (Fmoc). The need for such protection is readily determined by one skilled in the art. For a general description of protecting groups and their use, see T. W. Greene, Protective Groups in Organic Synthesis, John Wiley & Sons, New York, 1991.

### F. METHODS OF USE

The compounds and compositions disclosed herein can also be used in methods for treating various diseases and/or disorders that have been identified as being associated with any members of the IP6K family or IPMK.

The diseases and disorders to be treated include, but are not limited to, degenerative disorders, cancer, diabetes, autoimmune disorders, cardiovascular disorders, clotting disorders, diseases of the eye, infectious disease, and diseases caused by mutations in one or more genes.

More particularly, the diseases and disorders may include diabetes (e.g., type 1 diabetes, type 2 diabetes, gestational diabetes, obese diabetes), cardiovascular disease (e.g., cardiac failure, arrhythmia, ischemic cardiac diseases, heart valvular disease, arteriosclerosis), obesity (e.g., malignant mastocytosis, exogenous obesity, hyperinsulinar obesity, hyperplasmic obesity, hypophyseal adiposity, hypoplasmic obesity, hypothyroid obesity, hypothalamic obesity, symptomatic obesity, infantile obesity, upper body obesity, alimentary obesity, hypogonadal obesity, systemic mastocytosis, simple obesity, central obesity etc.), non-alcoholic fatty liver diseases (NAFLD), non-alcoholic steatohepatitis (NASH), hyperphagia, hyperlipidemia/dyslipidemia (e.g., hypertriglyceridemia, hypercholesterolemia, high LDL-cholesterolemia, hypo HDL-cholesterolemia, postprandial hyperlipemia), Hyperphosphatemia, hypophosphatemia, hyperkalemia, hypertension, diabetic complications (e.g., neuropathy, nephropathy, retinopathy, diabetic cardiomyopathy, cataract, macroangiopathy, osteopenia, hyperosmolar diabetic coma, infections (e.g., respiratory infection, urinary tract infection, gastrointestinal infection, dermal soft tissue infections, inferior limb infection), diabetic gangrene, xerostomia hypacusis, hypacusis, cerebrovascular diseases, peripheral blood circulation disorder), metabolic syndrome (pathology with not less than 3 selected from hyper-triglycerid(TG)emia, hypo HDL cholesterol(HDL-C)emia, hyper tension, abdomen obesity and impaired glucose tolerance), sarcopenia, emotional disorder, sexual dysfunction, depression, anxiety, neurosis, arteriosclerosis, gonitis, acute renopathy, glaucoma, ischemic disease, myocardial infarction, cerebral apoplexy, dementia, neurodegenerative diseases (e.g., amyotrophic lateral sclerosis), mitochondria disease, retinitis pigmentosa, glaucoma, Osteoporosis, fungi infections (i.e., yeast strains such as, but not limited to, Cryptococcus neoformans and/or Candida albicans), parasitic infections (i.e., Toxoplasma gondii) and the like.

In accordance with the purpose(s) of the currently disclosed subject matter, as embodied and broadly described herein, in one aspect relates to a method of preventing or treating a disease or disorder mediated by IP6K, the method comprising administering a therapeutically effective amount of a compound of Formula II to a subject in need thereof, wherein the compound of Formula II is:

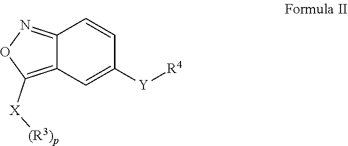

wherein X is aryl or heteroaryl; R³ is, in each instance, selected from the group consisting of alkyl, cycloalkyl, heterocycloalkyl, aryl, heteroaryl, halo, -D, —OH, —OR⁶, —N—COR⁶, —CONR⁷R⁸, —SO₂—NHR⁹, and NR¹⁰R¹¹;
p is an integer from 0 to 5;
Y is present or absent, and when present selected from the group consisting of alkyl, cycloalkyl, and alkenyl;
R⁴ is selected from the group consisting of —CO₂H, —CO₂R⁶, —CONR⁷R⁸, —SO₂—NHR⁶, —SO₃H, —P(O)(OH)(OR⁹), tetrazolyl, triazolyl, and oxazol-2(3H)-only; and
R⁵ is halo or alkyl;
wherein R⁶ is selected from alkyl, cycloalkyl, aryl, and heteroaryl, and
R⁷ and R⁸ are each independently selected from hydrogen, alkyl, cycloalkyl, heterocycloalkyl, aryl, and heteroaryl,
wherein alkyl, alkenyl, cycloalkyl, heterocycloalkyl, aryl, and heteroaryl are optionally substituted, and a pharmaceutically acceptable salt thereof.
In an embodiment, the alkyl, alkenyl, cycloalkyl, heterocycloalkyl, aryl, and heteroaryl are optionally substituted with one or more groups independently selected from halo, alkyl, hydroxyl, cyano, F, Cl, Br, —CH₃, —CH₂CH₃, —CH(CH₃)₂, —CH₂CH(CH₃)₂, —CH₂NH₂, —CH₂NHCH₃, —CH₂N(CH₃)₂, —CH₂CH₂NH₂, —CH₂CH₂CH₂NH₂, —CH₂CH₂CH₂CH₂NH₂, —CH₂CH(CH₃)NH₂, —CH₂CONH₂, —CH₂OH, —CH₂CH₂OH, —C(CH₃)₂OH, —CH(OH)CH(CH₃)₂, —C(CH₃)₂CH₂OH, —CH₂C(CH₃)₂OH, —CH₂CH₂SO₂CH₃, —CN, —CF₃, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NO₂, —NH₂, —NHCH₃, —N(CH₃)₂, —NHCOCH₃, —N(CH₃)COCH₃, —NHS(O)₂CH₃, —NHCH₂CH₂NH₂, —NHCH₂CH₂CH₂NH₂, —NHCH₂CH₂CH₂CH₂NH₂, —N(CH₃)C(CH₃)₂CONH₂, —N(CH₃)CH₂CH₂S(O)₂CH₃, ═O, —OH, —OCH₃, —OCH₂CH₂OCH₃, —OCH₂CH₂NH₂, —S(O)₂N(CH₃)₂, —SCH₃, —CH₂OCH₃, —S(O)₂CH₃, cyclopropyl, cyclobutyl, cyclopentyl, cyclohexyl, cycloheptyl, azetidinyl, azepanyl, oxetanyl, pyrrolidinyl, piperazinyl, piperidinyl, (piperidin-4-yl)ethyl, pyranyl, (piperidin-4-ylmethyl), morpholinomethyl, and morpholino;
and a pharmaceutically acceptable salt thereof.

Particular embodiments of the subject matter described herein include:

Embodiment 1: A compound of Formula II:

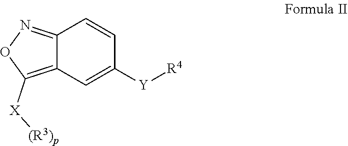

or a pharmaceutically acceptable salt thereof, wherein
X is aryl or heteroaryl;
R³ is, in each instance, selected from the group consisting of alkyl, cycloalkyl, heterocycloalkyl, aryl, heteroaryl, halo, -D, —OH, —OR⁵, —NH—COR⁶, —CONR⁷R⁸, —SO₂—NHR⁹, and NR¹⁰R¹¹;
p is an integer from 0 to 5;
Y is present or absent, and when present selected from the group consisting of alkyl, cycloalkyl, and alkenyl; and
R⁴ is selected from the group consisting of —CO₂H, —CO₂R⁵, —CONR⁷R⁸, —SO₂—NHR⁶, —SO₃H, —P(O)(OH)(OR⁹), heteroaryl, and heterocycloalkyl; wherein
R⁵ is selected from alkyl, aryl, heteroaryl, and heterocycloalkyl; R⁶ is selected from alkyl, cycloalkyl, aryl, and heteroaryl; R⁷ and R⁸ are each independently selected from hydrogen, alkyl, cycloalkyl, heterocycloalkyl, aryl, and heteroaryl; R⁹ is selected from alkyl, aryl, heteroaryl, and heterocycloalkyl; and R¹⁰ and R¹¹ are each independently selected from hydrogen, alkyl, cycloalkyl, heterocycloalkyl, aryl, and heteroaryl.

Embodiment 2: The compound of embodiment 1, wherein Y is absent.

Embodiment 3: The compound of embodiment 1 or 2, wherein R⁴ is selected from the group consisting of —CO₂H, heteroaryl, and heterocycloalkyl.

Embodiment 4: The compound of any one of the preceding embodiments, wherein R⁴ is selected from the group consisting of

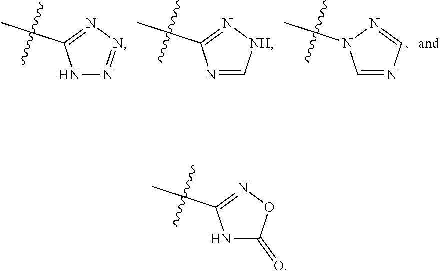 [SMILES: *c1nnn[nH]1 ; *c1nc[nH]n1 ; *n1cncn1 ; "," ; "and" ; *c1noc(=O)[nH]1]

Embodiment 5: The compound of embodiment 4, wherein R⁴ is

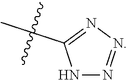 [SMILES: *c1nnn[nH]1]

Embodiment 6: The compound of embodiment 4, wherein R⁴ is

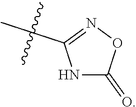 [SMILES: *c1noc(=O)[nH]1]

Embodiment 7: The compound of any one of embodiments 1 to 3, wherein R⁴ is —CO₂H.

Embodiment 8: The compound of any one of the preceding embodiments, wherein X is aryl.

Embodiment 9: The compound of embodiment 1, wherein the compound is selected from a compound of Formula III, IV, and V:

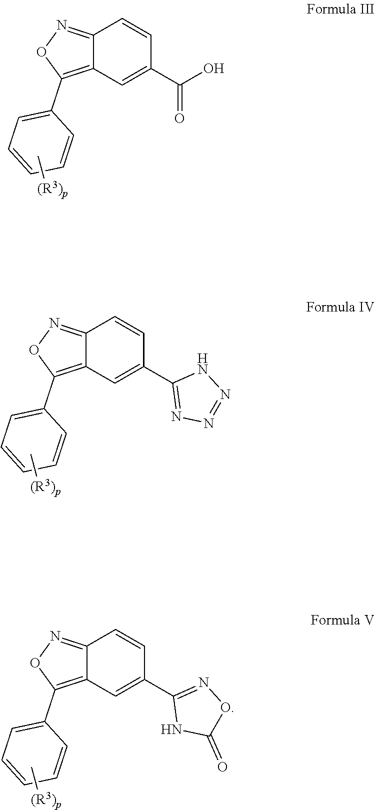

Embodiment 10: The compound of any one of embodiments 1 to 9, wherein R³ is C1-C6 alkyl, aryl or halo.

Embodiment 11: The compound of any one of embodiments 1 to 10, wherein R³ is methyl.

Embodiment 12: The compound of any one of embodiments 1 to 10, wherein R³ is 4-phenyl.

Embodiment 13: The compound of any one of embodiments 1 to 10, wherein R³ is selected from alkyl, aryl, heteroaryl, and heterocycloalkyl.

Embodiment 14: The compound of any one of the preceding embodiments, wherein p is 1.

Embodiment 15: The compound of any one of the preceding embodiments, wherein R³ is heterocycloalkyl.

Embodiment 16: The compound of any one of the preceding embodiments, wherein R³ is piperidinyl.

Embodiment 17: The compound of any one of the preceding embodiments, wherein R³ is —OR⁵.

Embodiments 18: The compound of any one of the preceding embodiments, wherein R⁵ is selected from alkyl, aryl, heteroaryl, and heterocycloalkyl.

Embodiments 19: The compound of any one of the preceding embodiments, wherein R⁵ is heteroaryl or heterocycloalkyl.

Embodiment 20: The compound of any one of the preceding embodiments, wherein R⁵ is pyridinyl or piperidinyl.

Embodiment 21: The compound of embodiment 19, wherein R⁵ is heterocycloalkyl.

Embodiment 22: The compound of embodiment 1, wherein X is phenyl, R³ is —NH—COR⁶, and p is 1, wherein R⁶ is selected from alkyl, cycloalkyl, aryl, heterocycloalkyl, and heteroaryl.

Embodiment 23: The compound of embodiment 22, wherein R⁶ is cycloalkyl.

Embodiment 24: The compound of embodiment 22, wherein R⁶ is heterocycloalkyl.

Embodiment 25: The compound of embodiment 22, wherein R⁶ is cyclopropyl or cyclobutyl.

Embodiment 26: A compound having one of the following structures:

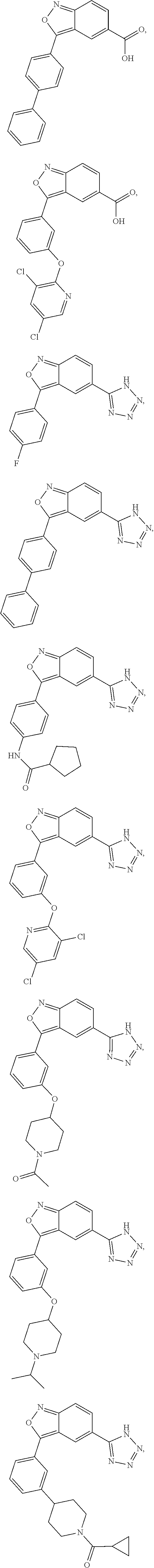

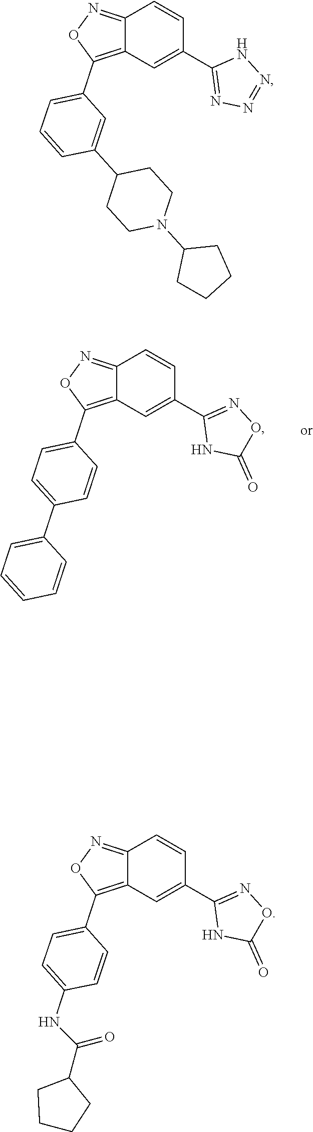 [SMILES: c1cc(-c2onc3ccc(-c4nnn[nH]4)cc23)cc(C2CCN(C3CCCC3)CC2)c1 ; O=c1[nH]c(-c2ccc3noc(-c4ccc(-c5ccccc5)cc4)c3c2)no1 ; O=C(Nc1ccc(-c2onc3ccc(-c4noc(=O)[nH]4)cc23)cc1)C1CCCC1]

Embodiment 27: A pharmaceutical composition comprising a compound according to any one of embodiments 1 to 26 or a pharmaceutically acceptable salt thereof, and one or more pharmaceutically acceptable carrier or excipient.

Embodiment 28: A method of preventing or treating a disease or disorder mediated by IP6K and/or IPMK, the method comprising administering a therapeutically effective amount of a compound of any one of embodiments 1 to 26 to a subject in need thereof.

Embodiment 29: The method of embodiment 28, wherein the disease or disorder is selected from obesity, obesity-related diseases, cancer, and viral infections.

Embodiment 30: The method of embodiment 28, wherein the obesity-related diseases are non-alcoholic fatty liver disease (NAFLD) or non-alcoholic steatohepatitis (NASH).

Embodiment 31: The method of embodiment 28, wherein the cancer is glioblastoma.

Embodiment 32: The method of embodiment 28, wherein the viral infection is coronavirus.

Embodiment 33: A kit for treating a disease or disorder mediated by IP6K and/or IPMK, the kit comprising: 1) a pharmaceutical composition comprising a compound of Formula II, and 2) instructions for use.

Embodiment 34: A compound of embodiment 1 for use as a medicament, and for use in treating a disease or disorder mediated by IP6K and/or IPMK.

### G. EXAMPLES

The following preparations and examples are given to enable those skilled in the art to more clearly understand and to practice the present invention. They should not be considered as limiting the scope of the invention, but merely as being illustrative and representative.

General Information: Microwave reactions were carried out using a CEM Discover-S reactor with a vertically focused IR external temperature sensor and an Explorer 72 autosampler. The dynamic mode was used to set up the desired temperature and hold time with the following fixed parameters: PreStirring, 1 min; Pressure, 200 psi; Power, 200 W; PowerMax, off; Stirring, high. Sonication was carried out on Branson 3510 Ultrasonic Cell. Centrifugation was carried out on Eppendorf Centrifuge 5418. Flash chromatography was carried out on Teledyne ISCO Combi Flash® Rf 200 with pre-packed silica gel disposable columns or pre-packed reverse phase C18 columns. Analytical HPLC was performed with prominence diode array detector (SPD-M20A). Samples were injected onto a 3.6 μm PEPTIDE XB—C18 100 Å, 150×4.6 mm LC column at room temperature. The flow rate was 1.0 mL/min. Various linear gradients were used with A being H₂O+0.1% TFA and B being acetonitrile+0.1% TFA. Analytical thin-layer chromatography (TLC) was performed with silica gel 60 F₂₅₄, 0.25 mm pre-coated TLC plates. TLC plates were visualized using UV₂₅₄ and phosphomolybdic acid with charring. All ¹H NMR spectra were obtained with a 400 or 500 MHz spectrometer using CDCl₃ (7.26 ppm), DMSO-d⁶ (2.50 ppm, quintet) or CD₃OD (3.31 ppm, quintet) as an internal reference. Signals are reported as m (multiplet), s (singlet), d (doublet), t (triplet), q (quartet), and bs (broad singlet); and coupling constants are reported in hertz (Hz). ¹³C NMR spectra were obtained with a 100 or 125 MHz spectrometer using CDCl₃ (77.2 ppm, triplet), DMSO-d⁶ (39.5 ppm, septet), or CD₃OD (49.3 ppm, septet) as the internal standard. LC/MS was performed using an analytical instrument with the UV detector set to 220 nm, 254 nm, and 280 nm, and a single quadrupole mass spectrometer using electrospray ionization (ESI) source. Samples were injected (2 L) onto a 4.6×50 mm, 1.8 μM, C18 column at room temperature. A linear gradient from 10% to 100% B (MeOH+0.1% acetic Acid) in 5.0 min was followed by pumping 100% B for another 2 or 4 min with A being H₂O+0.1% acetic acid. The flow rate was 1.0 mL/min. High-resolution (positive ion) mass spectra (HRMS) were acquired using a LCMS-TOF mass spectrometer. Purity of all final compounds (>95%) was determined by LC-MS.

### Example 1: (E)-N-Methyl-3-(3-phenylbenzo[c]isoxazol-5-yl)acrylamide and Ethyl (1R,2R)-2-(3-phenylbenzo[c]isoxazol-5-yl)cyclopropane-1-carboxylate

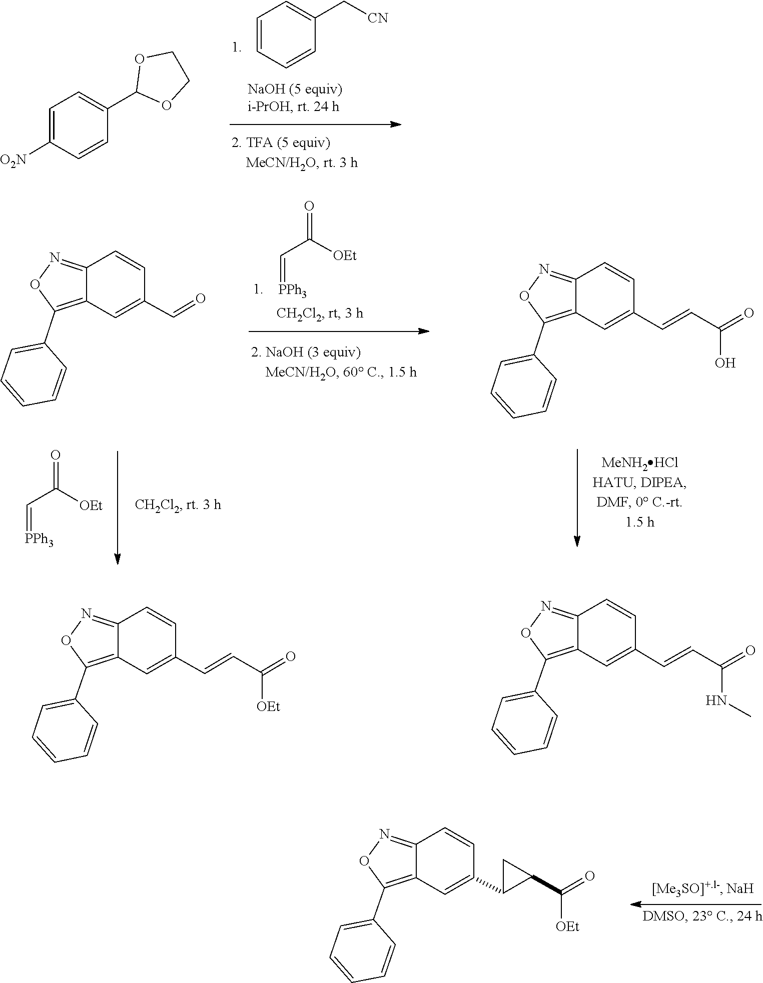

NaOH pellets (40.0 mg, 1.00 mmol) were granulated and added to i-PrOH (1.00 mL) to give a suspension. To the resulting mixture was added 2-(4-nitrophenyl)-1,3-dioxolane (39.0 mg, 0.200 mmol) and benzyl cyanide (57.7 μL, 0.500 mmol). The reaction mixture was stirred at room temperature overnight. Upon the completion of the reaction (monitored by LC-MS), the brownish precipitate was filtered and washed by a small amount of i-PrOH followed by plenty of water. The filter cake was collected and dried under reduced pressure to afford the 2,1-benzoisoxaole. Without further purification, the solid was added MeCN (1.5 mL) and water (0.5 mL). To the resulting suspension was added TFA (100 μL, 1.31 mmol). The reaction mixture was stirred at room temperature for 3 h, then neutralized by sat. NaHCO₃ solution and extracted with CH₂Cl₂ (3×). The combined organic phase was dried (Na₂SO₄) and concentrated under reduced pressure. The residue was purified by a silica gel column (hexane/EtOAc gradient) to afford 3-phenylbenzo[c]isoxazole-5-carbaldehyde as a yellow solid (36.7 mg, 0.164 mmol, 82%). ¹H NMR (400 MHz, CDCl₃) δ 10.01 (s, 1H), 8.40 (s, 1H), 8.12-8.04 (m, 2H), 7.84 (dd, J=9.3, 1.3 Hz, 1H), 7.69 (dt, J=8.7, 0.9 Hz, 1H), 7.65-7.59 (m, 3H); ¹³C NMR (126 MHz, CDCl₃) δ 190.52, 168.66, 158.61, 134.08, 131.74, 130.63, 129.75, 127.58, 127.48, 127.27, 116.82, 114.18; MS (ESI) for [M+H⁺] (C₁₄H₁₀NO₂⁺): calcd m/z 224.07; found m/z 224.10; LC-MS: 97% purity.

To a solution of 3-phenylbenzo[c]isoxazole-5-carbaldehyde (137 mg, 0.612 mmol) in CH₂Cl₂ (3.0 mL) was added ethyl (triphenylphosphoranylidene)acetate (224 mg, 0.642 mmol). The reaction mixture was stirred at room temperature for 3 hours and concentrated under reduced pressure. The residue was dissolved in MeCN (0.62 mL). Then, a solution of NaOH (73 mg, 1.8 mmol) in water (0.16 mL) was added. The reaction mixture was heated at 60° C. for 1.5 h, then the reaction was cooled down to room temperature and acidified by 1 M HCl to pH=1. The yellow precipitate was filtered and sonicated in minimum amount of 1:1 MeOH/H₂O to remove the trace amount of cis-isomer. The crude product was collected from centrifugation and further purified by a reverse phase column (MeCN/H₂O gradient) to afford (E)-3-(3-phenylbenzo[c]isoxazol-5-yl)acrylic acid as a yellow solid (141 mg, 0.532 mmol, 87%). ¹H NMR (500 MHz, DMSO-d₆) δ 8.43 (s, 1H), 8.17 (d, J=7.4 Hz, 2H), 7.85 (d, J=9.4 Hz, 1H), 7.75 (d, J=16.0 Hz, 1H), 7.71-7.58 (m, 4H), 6.62 (d, J=16.0 Hz, 1H); ¹³C NMR (214 MHz, DMSO-d₆) δ 167.73, 165.20, 157.27, 143.30, 131.70, 131.22, 129.74, 129.61, 127.13, 126.79, 124.09, 119.74, 115.73, 114.11; MS (ESI) for [M+H]⁺ (C₁₆H₁₂NO₃⁺): calcd. m/z 266.08; found m/z 266.10; LC-MS: 98% purity.

To a solution of (E)-3-(3-phenylbenzo[c]isoxazol-5-yl)acrylic acid (21.6 mg, 81.4 μmol) in DMF (0.81 mL) was added to DIPEA (43 μL, 0.24 mmol) and HATU (34.1 mg, 89.6 μmol) at 0° C. The mixture was stirred at 0° C. for 30 min, then methyl amine hydrochloride (8.2 mg, 0.12 mmol) was added. The resulting reaction mixture was stirred at room temperature for one hour, then quenched with water (2.5 mL). The product was crushed out and isolated by centrifugation. The crude product was washed with water (2×) and dried under lyophilization to afford (E)-N-methyl-3-(3-phenylbenzo[c]isoxazol-5-yl)acrylamide as a yellow solid (22.6 mg, 81.2 μmol, quantitative yield). ¹H NMR (400 MHz, CDCl₃) δ 8.04-7.97 (m, 2H), 7.89 (s, 1H), 7.69 (d, J=15.5 Hz, 1H), 7.63-7.49 (m, 5H), 6.40 (d, J=15.5 Hz, 1H), 5.80 (s, 1H), 2.97 (d, J=4.9 Hz, 3H); ¹³C NMR (126 MHz, CDCl₃) δ 166.44, 165.72, 157.83, 140.17, 131.74, 130.90, 129.56, 128.65, 128.12, 126.89, 122.98, 120.89, 116.41, 114.78, 26.78; MS (ESI) for [M+H]+(C₁₇H₁₅N₂O₂⁺): calcd. m/z 279.11; found m/z 279.10; LC-MS: 99% purity.

To an oven-dried round bottom flask was added a mixture of trimethylsulfonoxium iodide (139 mg, 0.630 mmol) and NaH (25.2 mg, 0.630 mmol, 60% wt suspension in mineral oil) in anhydrous DMSO (0.5 mL) at 0° C. The resulting suspension was stirred for additional 20 min. Then a solution of ethyl (E)-3-(3-(3,5-dimethylphenyl)benzo[c]isoxazol-5-yl)acrylate (96.4 mg, 0.300 mmol) in anhydrous DMSO (1.0 mL) was added to this suspension in dropwise. The reaction solution was then stirred at room temperature for 24 hours. Upon complete consumption of room temperature material, the resulting mixture was quenched by sat. NH₄C1. The mixture was extracted with EtOAc for three times, dried (Na₂SO₄) and concentrated under reduced pressure. The residue was purified by silica column chromatography to afford ethyl (1R,2R)-2-(3-phenylbenzo[c]isoxazol-5-yl)cyclopropane-1-carboxylate as a yellow solid (35.3 mg, 0.105 mmol, 35%). ¹H NMR (400 MHz, CDCl₃) δ 7.62-7.52 (m, 4H), 7.14 (d, J=2.0 Hz, 1H), 7.02 (dd, J=9.3, 1.6 Hz, 1H), 4.20 (q, J=7.1 Hz, 2H), 2.65-2.57 (m, 1H), 2.44 (s, 6H), 1.96 (ddd, J=8.5, 5.3, 4.2 Hz, 1H), 1.64 (dt, J=9.1, 5.0 Hz, 1H), 1.43-1.36 (m, 1H), 1.30 (t, J=7.1 Hz, 3H); MS (ESI) for [M+H]⁺ (C₂₁H₂₂NO₃⁺): calcd. m/z 336.16; found m/z 336.20; LC-MS: 95% purity.

Table 1 describes compounds prepared following procedures described in Example 1 using appropriate reagents. (Note: IC₅₀ (determined from enzyme-coupled assays): ++++ means <10 nM; +++ means between 10-100 nM, ++ means between 100 nM-1 μM; + means between 1-100 M; − means inactive).

| | | | | | | Physical Data |
|---|---|---|---|---|---|---|
| | | | | | | MS m/z |
| | | IP6K1 | IP6K2 | IP6K3 | IPMK | (M + 1) or and |
| Cmpd | Structure | IC₅₀ | IC₅₀ | IC₅₀ | IC₅₀ | ¹HNMR |
| 6 | 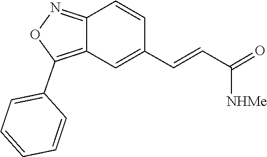 [SMILES: CNC(=O)/C=C/c1ccc2noc(-c3ccccc3)c2c1] | + | + | + | | ¹H NMR (400 MHz, CDCl₃) δ 8.04-7.97 (m, 2H), 7.89 (s, 1H), 7.69 (d, J = 15.5 Hz, 1H), 7.63-7.49 (m, 5H), 6.40 (d, J = 15.5 Hz, 1H), 5.80 (s, 1H), 2.97 (d, J = 4.9 Hz, 3H); ¹³C NMR (126 MHz, CDCl₃) δ 166.44, 165.72, 157.83, 140.17, 131.74, 130.90, 129.56, 128.65, |
| | | | | | | 128.12, 126.89, 122.98, |
| | | | | | | 120.89, 116.41, 114.78, |
| | | | | | | 26.78; MS (ESI) for [M + H]⁺ |
| | | | | | | (C₁₇H₁₅N₂O₂⁺): calcd. m/z |
| | | | | | | 279.11; found m/z 279.10. |
| 7 | 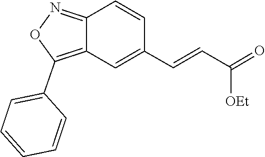 [SMILES: CCOC(=O)/C=C/c1ccc2noc(-c3ccccc3)c2c1] | + | + | | | ¹H NMR (400 MHz, DMSO-d₆) δ 8.52 (s, 1H), 8.23-8.18 (m, 2H), 7.91 (dd, J = 9.5, 1.3 Hz, 1H), 7.83 (d, J = 16.1 Hz, 1H), 7.70 (d, J = 9.5 Hz, 1H), 7.67-7.63 (m, 2H), 6.75 (d, J = 16.0 Hz, 1H), 6.52 (s, 1H), 4.22 (q, J = 7.1 Hz, 2H), 1.28 (t, J = 7.1 Hz, 3H). MS (ESI) for |
| | | | | | | [M + H]⁺ (C₁₈H₁₆NO₃⁺): |
| | | | | | | calcd. m/z 294.11; found |
| | | | | | | m/z 294.10. |
| 8 | 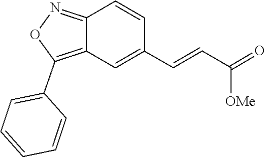 [SMILES: COC(=O)/C=C/c1ccc2noc(-c3ccccc3)c2c1] | + | + | + | | ¹H NMR (400 MHz, DMSO-d₆) δ 8.52 (s, 1H), 8.20 (dt, J = 8.5, 2.2 Hz, 2H), 7.94-7.80 (m, 2H), 7.71 (d, J = 9.4 Hz, 1H), 7.69- 7.61 (m, 3H), 6.76 (d, J = 16.0 Hz, 1H), 3.75 (s, 3H). MS (ESI) for [M + H]⁺ (C₁₇H₁₄NO₃⁺): calcd. m/z 280.10; found m/z 280.10. |
| 9 | 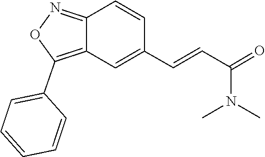 [SMILES: CN(C)C(=O)/C=C/c1ccc2noc(-c3ccccc3)c2c1] | + | + | − | | ¹H NMR (400 MHz, DMSO-d₆) δ 8.39 (d, J = 1.3 Hz, 1H), 8.19 (dd, J = 8.2, 1.6 Hz, 2H), 7.99 (dd, J = 9.4, 1.4 Hz, 1H), 7.71 (d, J = 9.3 Hz, 1H), 7.680-7.60 (m, 4H), 7.28 (d, J = 15.4 Hz, 1H), 3.20 (s, 3H), 2.95 (s, 3H). MS (ESI) for [M + H]⁺ (C₁₈H₁₇N₂O₂⁺): calcd. m/z |
| | | | | | | 293.13; found m/z 293.10. |
| 10 | 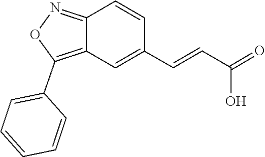 [SMILES: O=C(O)/C=C/c1ccc2noc(-c3ccccc3)c2c1] | ++ | ++ | + | | ¹H NMR (400 MHz, DMSO-d₆) δ 8.44 (s, 1H), 7.96 (d, J = 9.0 Hz, 2H), 7.85 (dd, J = 9.5, 1.4 Hz, 1H), 7.78 (d, J = 16.0 Hz, 1H), 7.67 (d, J = 9.4 Hz, 1H), 7.52 (t, J = 7.6 Hz, 1H), 7.41 (d, J = 7.6 Hz, 1H), 6.61 (d, J = 16.0 Hz, 1H), 2.46 (s, 3H); ¹³C NMR |
| | | | | | | (101 MHz, DMSO-d₆) δ 167.70, |
| | | | | | | 165.23, 157.19, 143.27, |
| | | | | | | 139.23, 131.83, 131.56, |
| | | | | | | 129.54, 129.38, 127.05, |
| | | | | | | 126.93, 124.28, 123.89, |
| | | | | | | 119.60, 115.63, 113.98, |
| | | | | | | 20.94; MS (ESI) for [M + H]⁺ |
| | | | | | | (C₁₇H₁₄NO₃⁺): calcd. m/z |
| | | | | | | 280.10; found m/z 280.10. |
| 11 | 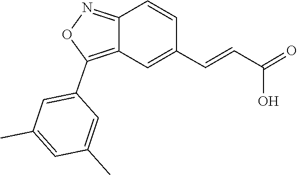 [SMILES: Cc1cc(C)cc(-c2onc3ccc(/C=C/C(=O)O)cc23)c1] | ++ | ++ | ++ | | ¹H NMR (400 MHz, DMSO-d₆) δ 8.46 (s, 1H), 7.88-7.82 (m, 1H), 7.78 (d, J = 2.5 Hz, 2H), 7.67 (d, J = 9.4 Hz, 1H), 7.24 (s, 1H), 6.61 (d, J = 16.0 Hz, 1H), 2.42 (s, 6H). ¹³C NMR (101 MHz, DMSO-d₆) δ 167.72, 165.40, 157.19, 143.38, 139.07, 132.67, 131.48, 129.25, 127.00, 124.55, |
| | | | | | | 124.20, 119.44, 115.63, |
| | | | | | | 113.91, 20.84. MS (ESI) for |
| | | | | | | [M + H]⁺ (C₁₈H₁₆NO₃⁺): |
| | | | | | | calcd. m/z 294.11; found |
| | | | | | | m/z 294.10. |
| 12 | 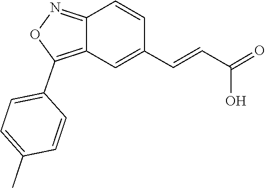 [SMILES: Cc1ccc(-c2onc3ccc(/C=C/C(=O)O)cc23)cc1] | ++ | ++ | | | ¹H NMR (400 MHz, DMSO-d₆) δ 8.43 (d, J = 1.5 Hz, 1H), 8.09 (d, J = 8.2 Hz, 2H), 7.85 (dd, J = 9.5, 1.3 Hz, 1H), 7.75 (d, J = 16.0 Hz, 1H), 7.67 (d, J = 9.4 Hz, 1H), 7.46 (d, J = 8.0 Hz, 2H), 6.62 (d, J = 16.0 Hz, 1H), 2.43 (s, 3H); MS (ESI) for [M + H]⁺ (C₁₇H₁₄NO₃⁺): calcd. m/z 280.10; found m/z 280.10. |
| 13 | 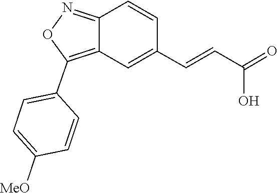 [SMILES: COc1ccc(-c2onc3ccc(/C=C/C(=O)O)cc23)cc1] | ++ | + | | | ¹H NMR (400 MHz, DMSO-d₆) δ 8.41 (s, 1H), 8.15 (d, J = 8.6 Hz, 2H), 7.83 (d, J = 9.4 Hz, 1H), 7.74 (d, J = 16.0 Hz, 1H), 7.64 (d, J = 9.4 Hz, 1H), 7.18 (d, J = 8.8 Hz, 2H), 6.60 (d, J = 16.0 Hz, 1H), 3.89 (s, 3H). ¹³C NMR (101 MHz, DMSO-d₆) δ 167.71, 165.31, 161.46, 157.14, 143.25, 130.99, 129.46, |
| | | | | | | 128.56, 124.33, 119.79, |
| | | | | | | 119.35, 115.40, 115.14, |
| | | | | | | 113.14, 55.57. MS (ESI) for |
| | | | | | | [M + H]⁺ (C₁₇H₁₄NO₄⁺): |
| | | | | | | calcd. m/z 296.09; found |
| | | | | | | m/z 296.10. |
| 14 | 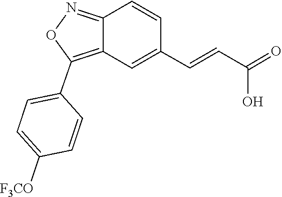 [SMILES: O=C(O)/C=C/c1ccc2noc(-c3ccc(OC(F)(F)F)cc3)c2c1] | ++ | ++ | + | | ¹H NMR (400 MHz, DMSO-d₆) δ 8.43 (s, 1H), 8.34-8.27 (m, 2H), 7.86 (dd, J = 9.5, 1.4 Hz, 1H), 7.75- 7.67 (m, 2H), 7.61 (dt, J = 7.9, 1.1 Hz, 2H), 6.65 (d, J = 16.0 Hz, 1H); ¹³C NMR (101 MHz, DMSO-d₆) δ 167.72, 163.62, 157.23, 149.80 (q, J = 1.7 Hz), 142.74, 132.09, 129.79, 128.96, 126.18, 123.44, 121.98, 120.43, |
| | | | | | | 120.04 (q, J = 259 Hz), |
| | | | | | | 115.70, 114.36; MS (ESI) for |
| | | | | | | [M + H]⁺ (C₁₇H₁₁F₃NO₄⁺): |
| | | | | | | calcd. m/z 350.06; |
| | | | | | | found m/z 350.05. |
| 15 | 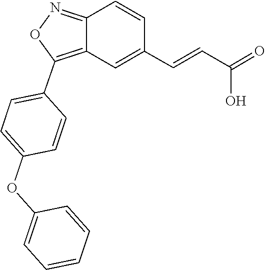 [SMILES: O=C(O)/C=C/c1ccc2noc(-c3ccc(Oc4ccccc4)cc3)c2c1] | ++ | +++ | ++ | | ¹H NMR (400 MHz, DMSO-d₆) δ 12.42 (s, 1H), 8.42 (s, 1H), 8.21 (d, J = 8.9 Hz, 2H), 7.85 (dd, J = 9.5, 1.4 Hz, 1H), 7.77-7.63 (m, 2H), 7.49 (dd, J = 8.6, 7.3 Hz, 2H), 7.31-7.23 (m, 1H), 7.24-7.12 (m, 4H), 6.62 (d, J = 16.0 Hz, 1H); ¹³C NMR (101 MHz, DMSO-d₆) δ 167.63, 164.76, 159.49, 157.14, 155.16, 143.19, 131.30, 130.37, 129.58, 128.87, 124.73, 124.03, 121.83, 119.95, 119.50, 118.45, 115.49, 113.54; MS (ESI) for [M + H]⁺ |
| | | | | | | (C₂₂H₁₆NO₄⁺): calcd. m/z |
| | | | | | | 358.11; found m/z 358.10. |
| 16 | 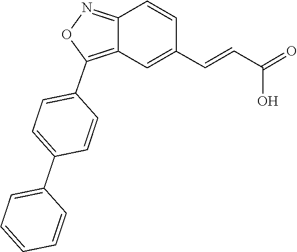 [SMILES: O=C(O)/C=C/c1ccc2noc(-c3ccc(-c4ccccc4)cc3)c2c1] | +++ | +++ | ++ | | ¹H NMR (400 MHz, DMSO-d₆) δ 8.51 (s, 1H), 8.29 (d, J = 8.2 Hz, 2H), 7.94 (d, J = 8.3 Hz, 2H), 7.88 (d, J = 9.5 Hz, 1H), 7.84-7.67 (m, 4H), 7.54 (t, J = 7.5 Hz, 2H), 7.45 (t, J = 7.3 Hz, 1H), 6.65 (d, J = 16.0 Hz, 1H); ¹³C NMR (101 MHz, DMSO-d₆) δ 167.64, 164.77, 157.22, 143.19, 142.43, 138.84, 131.66, 129.60, 129.19, 128.35, 127.72, 127.28, 126.86, 126.02, 124.05, 119.74, 115.65, 114.16; MS |
| | | | | | | (ESI) for [M + H]⁺ |
| | | | | | | (C₂₂H₁₆NO₃⁺): calcd. m/z |
| | | | | | | 342.11; found m/z 342.10. |
| 17 | 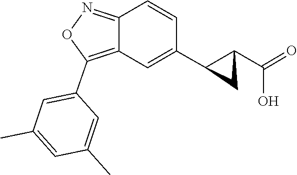 [SMILES: Cc1cc(C)cc(-c2onc3ccc([C@@H]4C[C@H]4C(=O)O)cc23)c1] | +++ | +++ | + | | ¹H NMR (400 MHz, CDCl₃) δ 7.63-7.52 (m, 4H), 7.14 (dt, J = 2.0, 1.0 Hz, 1H), 7.04 (dd, J = 9.1, 1.6 Hz, 1H), 2.75-2.66 (m, 1H), 2.45 (s, 6H), 1.98 (ddd, J = 8.4, 5.4, 4.2 Hz, 1H), 1.72 (dt, J = 9.6, 5.0 Hz, 1H), 1.49 (ddd, J = 8.4, 6.7, 4.8 Hz, 1H). ¹³C NMR (101 MHz, CDCl₃) δ 179.00, |
| | | | | | | 164.60, 157.51, 139.13, |
| | | | | | | 135.52, 132.28, 130.26, |
| | | | | | | 128.32, 124.52, 117.70, |
| | | | | | | 116.19, 114.35, 27.47, |
| | | | | | | 23.14, 21.58, 16.87. MS |
| | | | | | | (ESI) for [M + H]⁺ |
| | | | | | | (C₁₉H₁₈NO₃⁺): calcd. m/z |
| | | | | | | 308.13; found m/z 308.10. |

### Example 2: 3-(m-Tolyl)benzo[c]isoxazole-5-carboxylic Acid

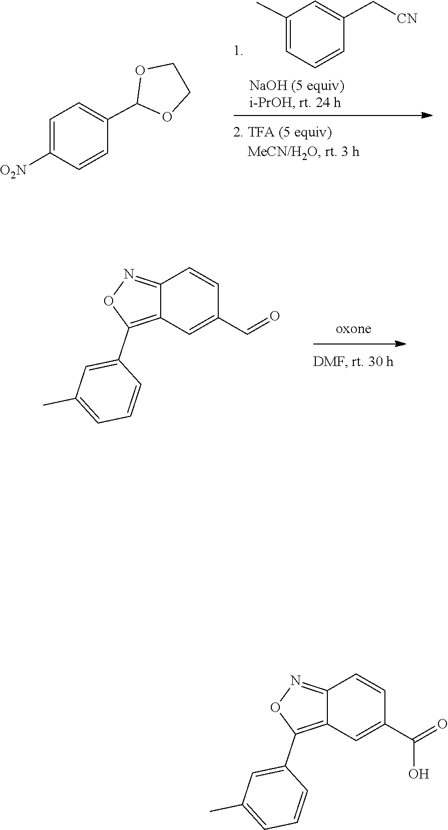

NaOH pellets (60.0 mg, 1.50 mmol) were granulated and added to i-PrOH (3.00 mL) to give a suspension. To the resulting mixture was added 2-(4-nitrophenyl)-1,3-dioxolane (58.6 mg, 0.300 mmol) and 2-(m-tolyl)acetonitrile (98 μL, 0.75 mmol). The reaction mixture was stirred at room temperature overnight. Upon the completion of the reaction (monitored by LC-MS), the brownish precipitate was filtered and washed by a small amount of i-PrOH followed by plenty of water. The filter cake was collected and dried under reduced pressure to afford the 2,1-benzoisoxaole intermediate. Without further purification, the solid was added MeCN (2.0 mL) and water (1.0 mL). To the resulting suspension was added TFA (138 μL, 1.80 mmol). The reaction mixture was stirred at room temperature for 3 h, then neutralized by sat. NaHCO₃ solution and extracted with CH₂Cl₂ (3×). The combined organic phase was dried (Na₂SO₄) and concentrated under reduced pressure. The residue was purified by a silica gel column (hexane/EtOAc gradient) to afford 3-(m-tolyl)benzo[c]isoxazole-5-carbaldehyde as a yellow solid (55.0 mg, 0.232 mmol, 77%). ¹H NMR (400 MHz, CDCl₃) δ 10.01 (s, 1H), 8.39 (t, J=1.2 Hz, 1H), 7.90-7.80 (m, 3H), 7.68 (dt, J=9.3, 0.9 Hz, 1H), 7.51 (t, J=8.0 Hz, 1H), 7.43-7.38 (m, 1H), 2.51 (s, 3H); MS (ESI) for [M+H]⁺ (C₁₅H₁₂NO₂⁺): calcd. m/z 238.09; found m/z 238.10.

To a solution of 3-(m-tolyl)benzo[c]isoxazole-5-carbaldehyde (23.7 mg, 0.100 mmol) in N,N-dimethylformamide (1.0 mL) was added Oxone triple salt (61.5 mg, 0.100 mmol). The reaction mixture was stirred at room temperature for 18 hours. Upon the completion of the reaction (monitored by LC-MS), the reaction mixture was purified by a reverse phase column to afford 3-(m-tolyl)benzo[c]isoxazole-5-carboxylic acid as a yellow solid (21.3 mg, 84.1 μmol, 84%). ¹H NMR (400 MHz, DMSO-d₆) δ 13.39 (s, 1H), 8.63 (d, J=1.4 Hz, 1H), 7.96-7.90 (m, 2H), 7.87 (dd, J=9.4, 1.4 Hz, 1H), 7.73 (d, J=9.4 Hz, 1H), 7.57 (t, J=7.9 Hz, 1H), 7.46 (d, J=7.6 Hz, 1H), 2.47 (s, 3H); ¹³C NMR (101 MHz, DMSO-d₆) δ 167.19, 166.53, 157.49, 139.34, 132.26, 130.47, 129.74, 127.53, 127.18, 126.80, 125.32, 124.21, 115.12, 113.40, 20.96; MS (ESI) for [M+H]⁺ (C₁₅H₁₂NO₃⁺): calcd. m/z 254.08; found m/z 254.10.

Table 2 describes compounds prepared following procedures described in Example 2 using appropriate reagents. (Note: IC₅₀ (determined from enzyme-coupled assays): ++++ means <10 nM; +++ means between 10-100 nM, ++ means between 100 nM-1 μM; + means between 1-100 M; − means inactive).

| | | | | | | Physical Data |
|---|---|---|---|---|---|---|
| | | IP6K1 | IP6K2 | IP6K3 | IPMK | MS m/z (M + 1) or and |
| Cmpd | Structure | IC₅₀ | IC₅₀ | IC₅₀ | IC₅₀ | ¹HNMR |
| 18 | 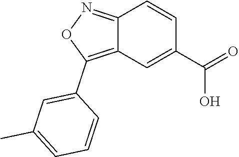 [SMILES: Cc1cccc(-c2onc3ccc(C(=O)O)cc23)c1] | ++ | ++ | + | − | ¹H NMR (400 MHz, DMSO-d₆) δ 13.39 (s, 1H), 8.63 (d, J = 1.4 Hz, 1H), 7.96-7.90 (m, 2H), 7.87 (dd, J = 9.4, 1.4 Hz, 1H), 7.73 (d, J = 9.4 Hz, 1H), 7.57 (t, J = 7.9 Hz, 1H), 7.46 (d, J = 7.6 Hz, 1H), 2.47 (s, 3H); ¹³C NMR |
| | | | | | | (101 MHz, DMSO-d₆) |
| | | | | | | δ 167.19, 166.53, |
| | | | | | | 157.49, 139.34, |
| | | | | | | 132.26, 130.47, |
| | | | | | | 129.74, 127.53, |
| | | | | | | 127.18, 126.80, |
| | | | | | | 125.32, 124.21, |
| | | | | | | 115.12, 113.40, 20.96; |
| | | | | | | MS (ESI) for [M + H]⁺ |
| | | | | | | (C₁₅H₁₂NO₃⁺): calcd. |
| | | | | | | m/z 254.08; found m/z |
| | | | | | | 254.10. |
| 19 | 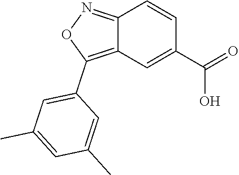 [SMILES: Cc1cc(C)cc(-c2onc3ccc(C(=O)O)cc23)c1] | +++ | ++ | + | ++ | ¹H NMR (400 MHz, DMSO-d₆) δ 8.59 (s, 1H), 7.85 (dd, J = 9.4, 1.4 Hz, 1H), 7.70 (dd, J = 11.2, 1.5 Hz, 3H), 7.26 (s, 1H), 2.41 (s, 6H); ¹³C NMR (101 MHz, DMSO-d₆) δ 167.38, 166.62, 157.51, 139.23, 133.13, 130.49, |
| | | | | | | 127.45, 126.82, |
| | | | | | | 125.43, 124.51, |
| | | | | | | 115.12, 113.36, 20.90; |
| | | | | | | MS (ESI) for [M + H]⁺ |
| | | | | | | (C₁₆H₁₄NO₃⁺): calcd. |
| | | | | | | m/z 268.10; found m/z |
| | | | | | | 268.10. |
| 20 | 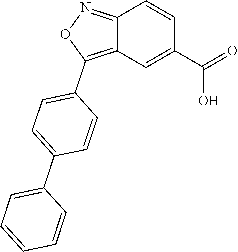 [SMILES: O=C(O)c1ccc2noc(-c3ccc(-c4ccccc4)cc3)c2c1] | +++ | +++ | ++ | +++ | ¹H NMR (400 MHz, DMSO-d₆) δ 13.30 (s, 1H), 8.69 (t, J = 1.2 Hz, 1H), 8.23 (d, J = 8.5 Hz, 2H), 8.03-7.95 (m, 2H), 7.88 (dd, J = 9.4, 1.3 Hz, 1H), 7.83- 7.73 (m, 3H), 7.54 (dd, J = 8.3, 6.8 Hz, 2H), 7.48-7.41 (m, 1H); ¹³C NMR (101 MHz, DMSO-d₆) δ 166.73, 166.54, 157.58, 142.86, 138.80, 130.55, 129.22, 128.45, 127.97, |
| | | | | | | 127.62, 127.54, |
| | | | | | | 128.45, 127.97, |
| | | | | | | 127.62, 127.54, |
| | | | | | | 126.89, 125.76, |
| | | | | | | 125.32, 115.19, |
| | | | | | | 113.59; MS (ESI) for |
| | | | | | | [M + H]⁺ |
| | | | | | | (C₂₀H₁₄NO₃⁺): calcd. |
| | | | | | | m/z 316.10; found m/z |
| | | | | | | 316.10. |
| 21 | 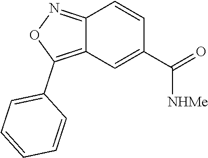 [SMILES: CNC(=O)c1ccc2noc(-c3ccccc3)c2c1] | + | + | + | − | ¹H NMR (400 MHz, CDCl₃) δ 8.37 (s, 1H), 8.05 (dd, J = 8.1, 1.6 Hz, 2H), 7.64 (d, J = 1.3 Hz, 2H), 7.62-7.50 (m, 3H), 6.24 (s, 1H), 3.06 (d, J = 4.8 Hz, 3H); ¹³C NMR (101 MHz, CDC1₃) δ 167.36, 167.16, |
| | | | | | | 157.96, 131.23, |
| | | | | | | 131.17, 129.57, |
| | | | | | | 128.81, 127.89, |
| | | | | | | 127.10, 121.83, |
| | | | | | | 116.14, 114.05, 27.18; |
| | | | | | | MS (ESI) for [M + H]⁺ |
| | | | | | | (C₁₅H₁₃N₂O₂⁺): calcd. |
| | | | | | | m/z 253.10; found m/z |
| | | | | | | 253.15. |
| 22 | 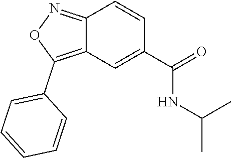 [SMILES: CC(C)NC(=O)c1ccc2noc(-c3ccccc3)c2c1] | + | ++ | + | − | ¹H NMR (400 MHz, CDCl₃) δ 8.37 (d, J = 1.3 Hz, 1H), 8.05 (dd, J = 8.1, 1.6 Hz, 2H), 7.67-7.50 (m, 5H), 6.00 (d, J = 6.6 Hz, 1H), 4.39-4.24 (m, 1H), 1.31 (d, J = 6.5 Hz, 6H). ¹³C NMR (101 MHz, CDC1₃) δ |
| | | | | | | 167.13, 165.83, |
| | | | | | | 157.97, 131.51, |
| | | | | | | 131.14, 129.57, |
| | | | | | | 128.80, 127.91, |
| | | | | | | 127.12, 121.87, |
| | | | | | | 116.03, 114.07, 42.42, |
| | | | | | | 22.99. MS (ESI) for |
| | | | | | | [M + H]⁺ |
| | | | | | | (C₁₇H₁₇N₂O₂⁺): calcd. |
| | | | | | | m/z 281.13; found m/z |
| | | | | | | 281.15. |
| 23 | 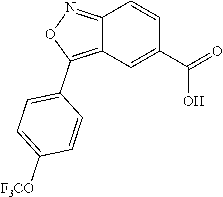 [SMILES: O=C(O)c1ccc2noc(-c3ccc(OC(F)(F)F)cc3)c2c1] | + | ++ | | | ¹H NMR (400 MHz, DMSO-d₆) δ 8.64 (d, J = 1.3 Hz, 1H), 8.31- 8.25 (m, 2H), 7.88 (dd, J = 9.4, 1.3 Hz, 1H), 7.76 (d, J = 9.4 Hz, 1H), 7.66 (d, J = 8.3 Hz, 2H). ¹³C NMR (101 MHz, DMSO-d₆) δ 166.47, 165.62, 157.56, 150.10 (q, J = 1.7 Hz), 130.58, |
| | | | | | | 129.31, 127.96, |
| | | | | | | 125.89, 125.06, |
| | | | | | | 122.12, 120.0 (q, J = |
| | | | | | | 259 Hz), 115.25, |
| | | | | | | 113.74. MS (ESI) for |
| | | | | | | [M + H]⁺ |
| | | | | | | (C₁₅H₉F₃NO₄⁺): calcd. |
| | | | | | | m/z 324.05; found m/z |
| | | | | | | 324.05. |
| 24 | 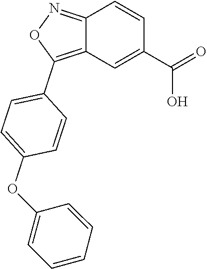 [SMILES: O=C(O)c1ccc2noc(-c3ccc(Oc4ccccc4)cc3)c2c1] | ++ | ++ | ++ | ++ | ¹H NMR (400 MHz, DMSO-d₆) δ 13.21 (d, J = 1H), 8.61 (d, J = 1.3 Hz, 1H), 8.18-8.10 (m, 2H), 7.85 (dd, J = 9.4, 1.4 Hz, 1H), 7.70 (dd, J = 9.4, 1.0 Hz, 1H), 7.48 (dd, J = 8.6, 7.3 Hz, 2H), 7.30-7.14 (m, 5H); ¹³C NMR (214 MHz, DMSO-d₆) δ 166.79, 166.64, 159.90, 157.58, 155.18, 130.59, 130.50, 129.26, 127.39, 125.46, 124.88, 121.62, |
| | | | | | | 120.03, 118.78, |
| | | | | | | 115.08, 113.07; MS |
| | | | | | | (ESI) for [M + H]⁺ |
| | | | | | | (C₂₀H₁₄NO₄⁺): calcd. |
| | | | | | | m/z 332.09; found m/z |
| | | | | | | 332.10. |
| 25 | 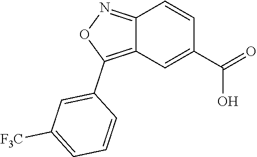 [SMILES: O=C(O)c1ccc2noc(-c3cccc(C(F)(F)F)c3)c2c1] | + | + | | + | ¹H NMR (400 MHz, DMSO-d₆) δ 13.36 (br, 1H), 8.65 (d, J = 1.3 Hz, 1H), 8.47 (d, J = 7.8 Hz, 1H), 8.36 (d, J = 2.4 Hz, 1H), 8.02 (d, J = 7.8 Hz, 1H), 7.96-7.87 (m, 2H), 7.79 (d, J = 9.4 Hz, 1H); MS (ESI) for |
| | | | | | | [M + H]⁺ |
| | | | | | | (C₁₅H₉F₃NO₃⁺): calcd. |
| | | | | | | m/z 308.05; found m/z |
| | | | | | | 308.10. |
| 26 | 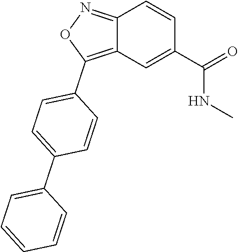 [SMILES: CNC(=O)c1ccc2noc(-c3ccc(-c4ccccc4)cc3)c2c1] | +++ | ++ | ++ | +++ | ¹H NMR (400 MHz, CDCl₃) δ 8.42 (t, J = (m, 2H), 7.84-7.78 (m, 1.2 Hz, 1H), 8.16-8.10 2H), 7.70-7.63 (m, 4H), 7.54-7.47 (m, 2H), 7.47-7.39 (m, 1H), 6.26 (s, 1H), 3.07 (d, J = 4.8 Hz, 3H). ¹³C NMR (101 MHz, CDCl₃) δ 167.37, 166.90, 157.99, 143.88, 139.85, 131.18, 129.20, 128.79, 128.39, 128.15, 127.47, |
| | | | | | | 127.28, 126.65, |
| | | | | | | 121.95, 116.15, |
| | | | | | | 114.12, 27.21. MS |
| | | | | | | (ESI) for [M + H]⁺ |
| | | | | | | (C₂₁H₁₇N₂O₂⁺): calcd. |
| | | | | | | m/z 329.13; found m/z |
| | | | | | | 329.10. |
| 27 | 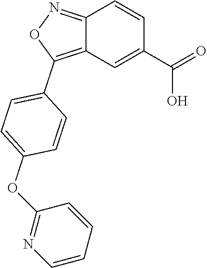 [SMILES: O=C(O)c1ccc2noc(-c3ccc(Oc4ccccn4)cc3)c2c1] | + | + | + | + | ¹H NMR (400 MHz, DMSO-d₆) δ 13.26 (br, 1H), 8.66 (d, J = 1.3 Hz, 1H), 8.27-8.14 (m, 3H), 7.94 (ddd, J = 9.2, 7.3, 2.0 Hz, 1H), 7.87 (dd, J = 9.4, 1.4 Hz, 1H), 7.74 (dd, J = 9.4, 1.0 Hz, 1H), 7.43 (d, J = 8.7 Hz, 2H), 7.25-7.17 (m, 2H). ¹³C NMR (101 MHz, DMSO-d₆) δ 166.68, 166.52, 162.29, 157.52, 156.59, 147.59, 140.62, 130.49, 128.73, |
| | | | | | | 127.40, 125.38, |
| | | | | | | 122.90, 122.11, |
| | | | | | | 119.90, 115.07, |
| | | | | | | 113.20, 112.29. MS |
| | | | | | | (ESI) for [M + H]⁺ |
| | | | | | | (C₁₉H₁₃N₂O₄⁺): calcd. |
| | | | | | | m/z 333.09; found m/z |
| | | | | | | 333.10. |
| 28 | 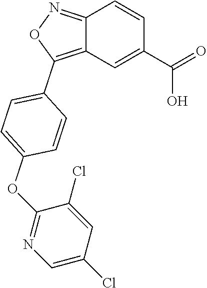 [SMILES: O=C(O)c1ccc2noc(-c3ccc(Oc4ncc(Cl)cc4Cl)cc3)c2c1] | ++ | ++ | + | + | ¹H NMR (400 MHz, DMSO-d₆) δ 8.65 (s, 1H), 8.42 (d, J = 2.3 Hz, 1H), 8.25-8.18 (m, 3H), 7.88 (dd, J = 9.4, 1.3 Hz, 1H), 7.75 (d, J = 9.4 Hz, 1H), 7.51 (d, J = 8.8 Hz, 2H); ¹³C NMR (214 MHz, DMSO-d₆) δ 166.50, 166.44, 157.52, 156.60, 155.56, 143.93, 139.62, 130.51, 128.78, 127.54, 125.98, 125.31, 123.76, 122.46, 119.09, 115.11, 113.36; MS |
| | | | | | | (ESI) for [M + H]⁺ |
| | | | | | | (C₁₉H₁₁C₁₂N₂O₄⁺): |
| | | | | | | calcd. m/z 401.01; |
| | | | | | | found m/z 401.00. |
| 29 | 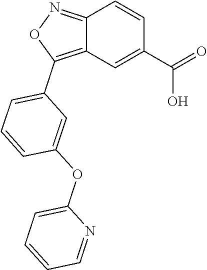 [SMILES: O=C(O)c1ccc2noc(-c3cccc(Oc4ccccn4)c3)c2c1] | ++ | ++ | + | + | ¹H NMR (400 MHz, DMSO-d₆) δ 13.29 (br, 1H), 8.61 (d, J = 1.3 Hz, 1H), 8.21 (dd, J = 5.1, 1.9 Hz, 1H), 7.98 (dt, J = 7.8, 1.3 Hz, 1H), 7.95-7.90 (m, 1H), 7.90-7.84 (m, 2H), 7.78-7.70 (m, 2H), 7.43 (dd, J = 7.6, 2.3 Hz, 1H), 7.22-7.15 (m, 2H); ¹³C NMR (101 MHz, DMSO-d₆) δ 166.44, 166.29, 162.53, 157.52, 154.60, 147.40, 140.52, 131.36, |
| | | | | | | 130.54, 127.98, |
| | | | | | | 127.76, 125.13, |
| | | | | | | 124.24, 123.09, |
| | | | | | | 119.59, 119.34, |
| | | | | | | 115.21, 113.76, |
| | | | | | | 112.01; MS (ESI) for |
| | | | | | | [M + H]⁺ |
| | | | | | | (C₁₉H₁₃N₂O₄⁺): calcd. |
| | | | | | | m/z 333.09; found m/z |
| | | | | | | 333.10. |
| 30 | 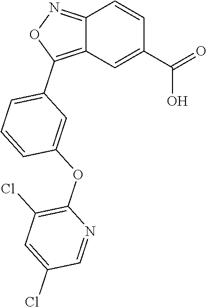 [SMILES: O=C(O)c1ccc2noc(-c3cccc(Oc4ncc(Cl)cc4Cl)c3)c2c1] | +++ | +++ | + | +++ | ¹H NMR (400 MHz, DMSO-d₆) δ 8.62 (s, 1H), 8.40 (d, J = 2.4 Hz, 1H), 8.21 (d, J = 2.3 Hz, 1H), 8.06-7.97 (m, 2H), 7.88 (dd, J = 9.4, 1.3 Hz, 1H), 7.80- 7.72 (m, 2H), 7.49 (dd, J = 8.1, 2.4 Hz, 1H); ¹³C NMR (126 MHz, DMSO-d₆) δ 166.48, 166.06, 157.51, 156.85, 153.78, 143.67, 139.46, 131.44, 130.58, 128.10, 127.92, 125.60, 125.12, 124.38, 123.93, 119.74, 118.82, |
| | | | | | | 115.17, 113.82; MS |
| | | | | | | (ESI) for [M + H]⁺ |
| | | | | | | (C₁₉H₁₁C₁₂N₂O₄⁺): |
| | | | | | | calcd. m/z 401.01; |
| | | | | | | found m/z 401.00. |
| 31 | 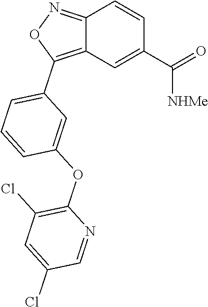 [SMILES: CNC(=O)c1ccc2noc(-c3cccc(Oc4ncc(Cl)cc4Cl)c3)c2c1] | ++ | ++ | | + | ¹H NMR (400 MHz, DMSO-d₆) δ 8.67 (d, J = 4.8 Hz, 1H), 8.49 (t, J = 1.3 Hz, 1H), 8.39 (d, J = 2.3 Hz, 1H), 8.21 (d, J = 2.3 Hz, 1H), 8.07 (dt, J = 8.1, 1.2 Hz, 1H), 7.99 (t, J = 2.0 Hz, 1H), 7.85 (dd, J = 9.4, 1.4 Hz, 1H), 7.79-7.70 (m, 2H), 7.47 (ddd, J = 8.2, 2.4, 0.9 Hz, 1H), 2.87-2.79 (d, J = 4.0 Hz, 3H); ¹³C NMR (101 MHz, DMSO-d₆) δ 165.82, 165.09, 157.38, 156.91, 153.85, 143.73, |
| | | | | | | 139.45, 131.41, |
| | | | | | | 131.27, 130.45, |
| | | | | | | 128.35, 125.59, |
| | | | | | | 124.14, 123.74, |
| | | | | | | 120.65, 119.53, |
| | | | | | | 118.79, 114.91, |
| | | | | | | 113.56, 26.23; MS |
| | | | | | | (ESI) for [M + H]⁺ |
| | | | | | | (C₂₀H₁₄C₁₂N₃O₃⁺): |
| | | | | | | calcd. m/z 414.04; |
| | | | | | | found m/z 414.05. |
| 32 | 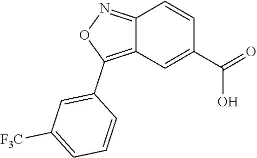 [SMILES: O=C(O)c1ccc2noc(-c3cccc(C(F)(F)F)c3)c2c1] | + | + | | + | ¹H NMR (400 MHz, DMSO-d₆) δ 13.36 (br, 1H), 8.65 (d, J = 1.3 Hz, 1H), 8.47 (d, J = 7.8 Hz, 1H), 8.36 (d, J = 2.4 Hz, 1H), 8.02 (d, J = 7.8 Hz, 1H), 7.96-7.87 (m, 2H), 7.79 (d, J = 9.4 Hz, 1H); MS (ESI) for |
| | | | | | | [M + H]⁺ |
| | | | | | | (C₁₅H₉F₃NO₃⁺): calcd. |
| | | | | | | m/z 308.05; found m/z |
| | | | | | | 308.10. |
| 33 | 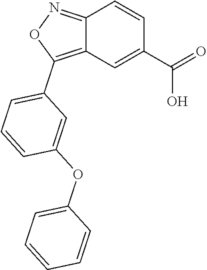 [SMILES: O=C(O)c1ccc2noc(-c3cccc(Oc4ccccc4)c3)c2c1] | ++ | ++ | ++ | +++ | ¹H NMR (400 MHz, DMSO-d₆) δ 13.29 (s, 1H), 8.54 (s, 1H), 7.93-7.83 (m, 2H), 7.77-7.66 (m, 2H), 7.62 (t, J = 2.0 Hz, 1H), 7.47 (t, J = 7.9 Hz, 2H), 7.30-7.20 (m, 2H), 7.17 (d, J = 7.9 Hz, 2H); ¹³C NMR (101 MHz, DMSO-d₆) δ 166.42, 166.18, 157.91, 157.52, 155.74, 131.79, 130.52, 130.36, 128.32, 127.82, 125.02, 124.39, |
| | | | | | | 121.78, 121.11, |
| | | | | | | 119.43, 115.81, |
| | | | | | | 115.25, 113.72; MS |
| | | | | | | (ESI) for [M + H]⁺ |
| | | | | | | (C₂₀H₁₄NO₄⁺): calcd. |
| | | | | | | m/z 332.09; found m/z |
| | | | | | | 332.10. |
| 34 | 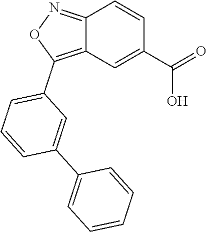 [SMILES: O=C(O)c1ccc2noc(-c3cccc(-c4ccccc4)c3)c2c1] | +++ | ++ | ++ | +++ | ¹H NMR (400 MHz, DMSO-d₆) δ 13.31 (br, 1H), 8.66 (s, 1H), 8.30 (d, J = 1.8 Hz, 1H), 8.14 (d, J = 8.0 Hz, 1H), 7.97-7.92 (m, 1H), 7.89 (dd, J = 9.4, 1.3 Hz, 1H), 7.86-7.71 (m, 4H), 7.59-7.50 (m, 2H), 7.45 (t, J = 7.3 Hz, 1H); ¹³C NMR (101 MHz, DMSO-d₆) δ 167.01, 166.51, 157.56, 141.68, 139.14, 130.57, |
| | | | | | | 129.92, 129.18, |
| | | | | | | 128.20, 127.65, |
| | | | | | | 127.52, 127.03, |
| | | | | | | 126.11, 125.34, |
| | | | | | | 124.99, 115.20, |
| | | | | | | 113.89, 113.72; MS |
| | | | | | | (ESI) for [M + H]⁺ |
| | | | | | | (C₂₀H₁₄NO₃⁺): calcd. |
| | | | | | | m/z 316.10; found m/z |
| | | | | | | 316.10. |
| 35 | 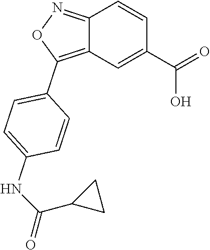 [SMILES: O=C(O)c1ccc2noc(-c3ccc(NC(=O)C4CC4)cc3)c2c1] | + | + | ++ | + | ¹H NMR (400 MHz, DMSO-d₆) δ 10.68 (s, 1H), 8.63 (s, 1H), 8.09 (d, J = 8.8 Hz, 2H), 7.94-7.85 (m, 3H), 7.67 (d, J = 9.3 Hz, 1H), 1.87 (tt, J = 7.3, 5.4 Hz, 1H), 0.90-0.80 (m, 4H); ¹³C NMR (126 MHz, DMSO-d₆) δ 172.34, 166.85, 166.73, 157.51, 142.21, 130.62, 127.83, 125.32, 121.23, 119.55, 118.25, 114.82, |
| | | | | | | 112.85, 14.77, 7.66. |
| | | | | | | MS (ESI) for [M + H]⁺ |
| | | | | | | (C₁₈H₁₅N₂O₄⁺): calcd. |
| | | | | | | m/z 323.10. found m/z |
| | | | | | | 323.10. |
| 36 | 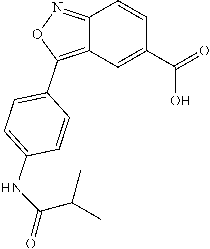 [SMILES: CC(C)C(=O)Nc1ccc(-c2onc3ccc(C(=O)O)cc23)cc1] | ++ | ++ | ++ | + | ¹H NMR (400 MHz, DMSO-d₆) δ 10.25 (s, 1H), 8.65 (t, J = 1.2 Hz, 1H), 8.10 (d, J = 9.4 Hz, 2H), 7.92 (d, J = 7.9 Hz, 2H), 7.85 (dd, J = 9.4, 1.3 Hz, 1H), 7.70 (dd, J = 9.4, 1.0 Hz, 1H), 2.66 (p, J = 6.8 Hz, 1H), 1.14 (d, J = 6.8 Hz, 7H); ¹³C NMR (126 MHz, DMSO-d₆) δ 175.89, 166.98, 166.58, 157.49, 142.39, 7130.45, |
| | | | | | | 127.81, 127.06, |
| | | | | | | 125.57, 121.22, |
| | | | | | | 119.69, 114.93, |
| | | | | | | 112.81, 35.12, 19.43. |
| | | | | | | MS (ESI) for [M + H]⁺ |
| | | | | | | (C₁₈H₁₇N₂O₄⁺): calcd. |
| | | | | | | m/z 325.12; found m/z |
| | | | | | | 325.10. |
| 37 | 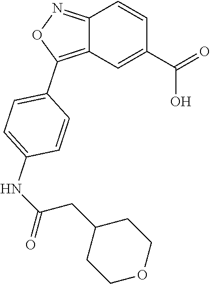 [SMILES: O=C(CC1CCOCC1)Nc1ccc(-c2onc3ccc(C(=O)O)cc23)cc1] | + | + | + | + | ¹H NMR (400 MHz, DMSO-d₆) δ 10.33 (s, 1H), 8.64 (s, 1H), 8.10 (d, J = 8.6 Hz, 2H), 7.91 (d, J = 8.7 Hz, 2H), 7.86 (dd, J = 9.4, 1.3 Hz, 1H), 7.69 (d, J = 9.4 Hz, 1H), 3.88- 3.80 (m, 2H), 2.32 (d, J = 7.1 Hz, 2H), 2.12- 1.96 (m, 1H), 1.65- 1.57 (m, 2H), 1.30- 1.22 (m, 2H). MS (ESI) for [M + H]⁺ (C₂₁H₂₁N₂O₅⁺): calcd. m/z 381.15; found m/z 381.20. |
| 38 | 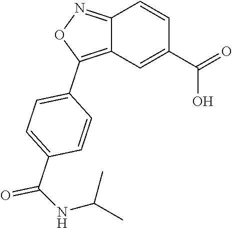 [SMILES: CC(C)NC(=O)c1ccc(-c2onc3ccc(C(=O)O)cc23)cc1] | ++ | ++ | ++ | + | ¹H NMR (400 MHz, DMSO-d₆) δ 8.67 (s, 1H), 8.46 (d, J = 7.7 Hz, 1H), 8.23 (d, J = 8.4 Hz, 2H), 8.10 (d, J = 8.4 Hz, 2H), 7.89 (dd, J = 9.4, 1.3 Hz, 1H), 7.78 (d, J = 9.4 Hz, 1H), 4.18-4.09 (m, 1H), 1.20 (d, J = 6.6 Hz, 7H); ¹³C NMR (126 MHz, DMSO-d₆) δ 166.50, 166.23, 164.51, 157.60, 137.03, 130.61, |
| | | | | | | 128.71, 128.64, |
| | | | | | | 128.01, 126.83, |
| | | | | | | 125.15, 115.33, |
| | | | | | | 113.98, 41.26, 22.32; |
| | | | | | | MS (ESI) for [M + H]⁺ |
| | | | | | | (C₁₈H₁₇N₂O₄⁺): calcd. |
| | | | | | | m/z 325.12; found m/z |
| | | | | | | 325.10. |
| 39 | 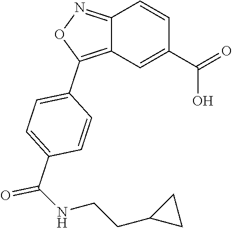 [SMILES: O=C(O)c1ccc2noc(-c3ccc(C(=O)NCCC4CC4)cc3)c2c1] | ++ | ++ | ++ | ++ | ¹H NMR (400 MHz, DMSO-d₆) δ 8.69 (d, J = 7.8 Hz, 2H), 8.23 (d, J = 8.3 Hz, 2H), 8.10 (d, J = 8.3 Hz, 2H), 7.89 (dd, J = 9.4, 1.3 Hz, 1H), 7.78 (d, J = 9.4 Hz, 1H), 1.47 (q, J = 7.1 Hz, 2H), 0.74 (ddt, J = 10.1, 7.2, 3.7 Hz, 1H), 0.47-0.40 (m, 2H), 0.12-0.06 (m, 2H), (two protons of CH₂ overlap with |
| | | | | | | the DMSO-d⁶ moister |
| | | | | | | peak at 3.36 ppm). ¹³C |
| | | | | | | NMR (126 MHz, |
| | | | | | | DMSO-d₆) 8 166.49, |
| | | | | | | 166.19, 165.20, |
| | | | | | | 157.58, 136.90, |
| | | | | | | 130.59, 128.73, |
| | | | | | | 128.50, 127.99, |
| | | | | | | 126.89, 125.15, |
| | | | | | | 115.30, 113.97, 34.00, |
| | | | | | | 8.68, 4.17. MS (ESI) |
| | | | | | | for [M + H]⁺ |
| | | | | | | (C₂₀H₁₉N₂O₄⁺): calcd. |
| | | | | | | m/z 351.13; found m/z |
| | | | | | | 351.10. |
| 40 | 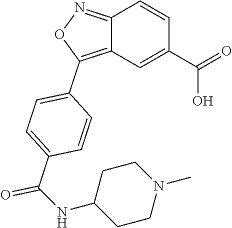 [SMILES: CN1CCC(NC(=O)c2ccc(-c3onc4ccc(C(=O)O)cc34)cc2)CC1] | + | + | | − | ¹H NMR (400 MHz, CD₃OD) δ 8.76 (s, Hz, 2H), 8.10 (d, J = 1H), 8.23 (d, J = 8.2 8.1 Hz, 2H), 7.97 (dd, J = 9.4, 1.3 Hz, 1H), 7.68 (d, J = 9.4 Hz, 1H), 4.28-4.15 (m, 1H), 3.70-3.54 (m, 2H), 3.31-3.15 (m, 2H), 2.92 (s, 3H), 2.36-2.21 (m, 2H), 2.10-1.88 (m, 2H). MS (ESI) for [M + H]⁺ |
| | | | | | | (C₂₁H₂₂N₃O₄⁺): calcd. |
| | | | | | | m/z 380.16; found m/z |
| | | | | | | 380.20. |
| 41 | 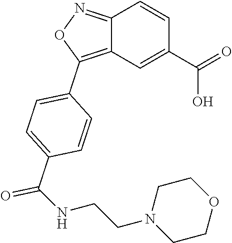 [SMILES: O=C(O)c1ccc2noc(-c3ccc(C(=O)NCCN4CCOCC4)cc3)c2c1] | − | − | | − | ¹H NMR (400 MHz, CD₃OD) δ 8.75 (d, J = 1.2 Hz, 1H), 8.28-8.23 (m, 2H), 8.16 (d, J = 8.5 Hz, 2H), 7.98 (dd, J = 9.4, 1.3 Hz, 1H), 7.69 (dd, J = 9.5, 1.0 Hz, 1H), 4.03-3.92 (m, 4H), 3.84 (t, J = 5.9 Hz, 2H), 3.53-3.36 (m, 4H), 3.44 (t, J = 6.1 Hz, 2H). ¹³C NMR (126 MHz, CD₃OD) δ 168.61, 167.38, 159.42, 137.66, |
| | | | | | | 136.75, 133.15, |
| | | | | | | 131.90, 131.70, |
| | | | | | | 129.82, 128.08, |
| | | | | | | 126.33, 116.28, |
| | | | | | | 115.77, 65.25, 58.60, |
| | | | | | | 53.70, 35.81. MS |
| | | | | | | (ESI) for [M + H]⁺ |
| | | | | | | (C₂₁H₂₂N₃O₅⁺): calcd. |
| | | | | | | m/z 396.16; found m/z |
| | | | | | | 396.20. |
| 42 | 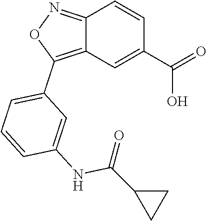 [SMILES: O=C(O)c1ccc2noc(-c3cccc(NC(=O)C4CC4)c3)c2c1] | ++ | ++ | + | +++ | ¹H NMR (500 MHz, DMSO-d₆) δ 10.58 (s, 1H), 8.65 (s, 1H), 8.44 (s, 1H), 7.91-7.72 (m, 4H), 7.60 (t, J = 8.0 Hz, 1H), 1.83 (td, J = 7.5, 3.8 Hz, 1H), 0.85 (t, J = 5.9 Hz, 4H). ¹³C NMR (126 MHz, DMSO-d₆) δ 172.23, 166.84, 166.50, 157.56, 140.49, 130.52, 130.43, 127.72, 127.18, |
| | | | | | | 125.09, 121.62, |
| | | | | | | 121.31, 116.56, |
| | | | | | | 115.27, 113.46, 14.78, |
| | | | | | | 7.50. MS (ESI) for |
| | | | | | | [M + H]⁺ |
| | | | | | | (C₁₈H₁₅N₂O₄⁺): calcd. |
| | | | | | | m/z 323.10; found m/z |
| | | | | | | 323.10. |
| 43 | 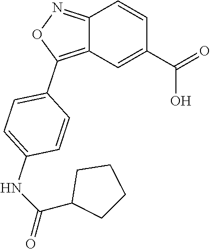 [SMILES: O=C(O)c1ccc2noc(-c3ccc(NC(=O)C4CCCC4)cc3)c2c1] | ++ | ++ | ++ | + | ¹H NMR (500 MHz, DMSO-d₆) δ 10.28 (s, 1H), 8.65 (s, 1H), 8.10 (d, J = 8.7 Hz, 2H), 2H), 7.86 (s, 1H), 7.69 7.92 (d, J = 8.7 Hz, (d, J = 9.4 Hz, 1H), 2.84 (p, J = 7.9 Hz, 1H), 1.95-1.82 (m, 2H), 1.80-1.64 (m, 4H), 1.65-1.52 (m, 2H). ¹³C NMR (126 MHz, DMSO-d₆) δ 175.08, 167.00, 166.60, 157.50, 142.41, 130.46, |
| | | | | | | 127.82, 127.06, |
| | | | | | | 125.59, 121.19, |
| | | | | | | 119.67, 114.94, |
| | | | | | | 112.82, 45.46, 30.09, |
| | | | | | | 25.71. MS (ESI) for |
| | | | | | | [M + H]⁺ |
| | | | | | | (C₂₀H₁₉N₂O₄⁺): calcd. |
| | | | | | | m/z 351.13; found m/z |
| | | | | | | 351.10. |
| 44 | 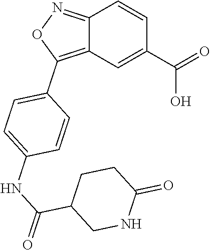 [SMILES: O=C1CCC(C(=O)Nc2ccc(-c3onc4ccc(C(=O)O)cc34)cc2)CN1] | + | + | + | + | ¹H NMR (500 MHz, DMSO-d₆) δ 10.47 (s, 1H), 8.64 (s, 1H), 8.11 (d, J = 8.5 Hz, 2H), 7.91 (d, J = 8.6 Hz, 2H), 7.85 (d, J = 9.4 Hz, 1H), 7.70 (d, J = 9.3 Hz, 1H), 7.54 (d, J = 3.3 Hz, 1H), 2.84 (dt, J = 10.3, 5.3 Hz, 1H), 2.32-2.19 (m, 2H), 2.10-1.99 (m, 1H), 1.97-1.86 (m, 1H), (two protons of CH₂ overlap with the DMSO-d₆ moister |
| | | | | | | peak at 3.37 ¹³C |
| | | | | | | NMR (126 MHz, |
| | | | | | | DMSO-d⁶) δ 171.99, |
| | | | | | | 169.70, 166.88, |
| | | | | | | 166.61, 157.51, |
| | | | | | | 141.99, 130.50, |
| | | | | | | 127.86, 127.20, |
| | | | | | | 125.51, 121.54, |
| | | | | | | 119.81, 114.96, |
| | | | | | | 112.90, 43.11, 40.27, |
| | | | | | | 30.17, 24.27. MS |
| | | | | | | (ESI) for [M+H]+ |
| | | | | | | (C₂₀H₁₈N₃O₅⁺): calcd. |
| | | | | | | m/z 380.12; found m/z |
| | | | | | | 380.10. |
| 45 | 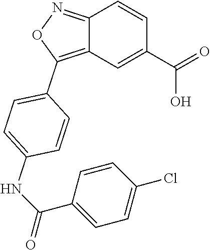 [SMILES: O=C(O)c1ccc2noc(-c3ccc(NC(=O)c4ccc(Cl)cc4)cc3)c2c1] | ++ | ++ | + | + | ¹H NMR (400 MHz, DMSO-d₆) δ 10.69 (s, 1H), 8.68 (s, 1H), 8.17 (d, J = 8.7 Hz, 2H), 8.10 (d, J = 8.9 Hz, 2H), 8.06-7.98 (m, 3H), 7.86 (d, J = 9.4 Hz, 1H), 7.71 (d, J = 9.4 Hz, 1H), 7.64 (d, J = 8.5 Hz, 2H). ¹³C NMR (126 MHz, DMSO-d₆) δ 166.89, 166.61, 164.96, 157.54, 142.00, 136.80, 133.29, 130.49, 129.84, |
| | | | | | | 128.59, 127.74, |
| | | | | | | 127.21, 125.56, |
| | | | | | | 122.00, 120.89, |
| | | | | | | 115.01, 113.00. MS |
| | | | | | | (ESI) for [M + H]⁺ |
| | | | | | | (C₂₁H₁₄CIN₂O₄⁺): |
| | | | | | | calcd. m/z 393.06; |
| | | | | | | found m/z 393.10. |
| 46 | 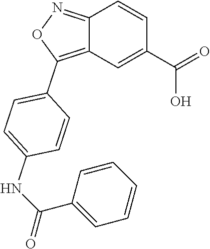 [SMILES: O=C(O)c1ccc2noc(-c3ccc(NC(=O)c4ccccc4)cc3)c2c1] | ++ | ++ | ++ | + | ¹H NMR (400 MHz, DMSO-d₆) δ 10.64 (s, 1H), 8.68 (s, 1H), 8.17 (d, J = 9.2 Hz, 2H), 8.12 (d, J = 9.2 Hz, 2H), 8.00 (d, J = 7.6 Hz, 2H), 7.86 (d, J = 9.4 Hz, 1H), 7.71 (d, J = 9.3 Hz, 1H), 7.63 (t, J = 7.2 Hz, 1H), 7.56 (t, J = 7.5 Hz, 2H). ¹³C NMR (101 MHz, DMSO-d₆) δ 166.95, 166.62, 166.10, 157.54, 142.22, 134.62, 131.97, |
| | | | | | | 130.50, 128.52, |
| | | | | | | 127.87, 127.73, |
| | | | | | | 127.19, 125.58, |
| | | | | | | 121.86, 120.83, |
| | | | | | | 114.99, 112.98. MS |
| | | | | | | (ESI) for [M + H]⁺ |
| | | | | | | (C₂₁H₁₅N₂O₄⁺): calcd. |
| | | | | | | m/z 359.10; found m/z |
| | | | | | | 359.10. |
| 47 | 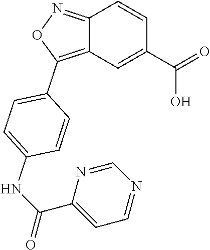 [SMILES: O=C(O)c1ccc2noc(-c3ccc(NC(=O)c4ccncn4)cc3)c2c1] | ++ | ++ | ++ | − | ¹H NMR (500 MHz, DMSO-d₆) δ 11.24 (s, 1H), 9.46 (d, J = 1.4 Hz, 1H), 9.16 (d, J = 5.1 Hz, 1H), 8.68 (s, 1H), 8.26 (d, J = 8.9 Hz, 2H), 8.22-8.17 (m, 3H), 7.87 (d, J = 9.4 Hz, 1H), 7.72 (d, J = 9.4 Hz, 1H). MS (ESI) for [M + H]⁺ (C₁₉H₁₃N₄0₄⁺): calcd. m/z 361.09; found m/z 361.10. |
| 48 | 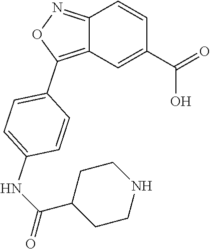 [SMILES: O=C(O)c1ccc2noc(-c3ccc(NC(=O)C4CCNCC4)cc3)c2c1] | + | + | | − | ¹H NMR (400 MHz, DMSO-d₆) δ 10.63 (s, 1H), 8.64 (s, 1H), 8.11 (d, J = 8.6 Hz, 2H), 7.94 (d, J = 8.6 Hz, 2H), 7.86 (d, J = 9.4 Hz, 1H), 7.69 (d, J = 9.4 Hz, 1H), 2.99-2.88 (m, 2H), 2.76 (t, J = 8.6 Hz, 1H), 2.06-1.96 (m, 2H), 1.94-1.80 (m, 2H), (two protons of CH₂ overlap with the DMSO-d⁶ moister peak at 3.37 ppm). MS (ESI) for [M + H]⁺ |
| | | | | | | (C₂₀H₂₀N₃O₄⁺): calcd. |
| | | | | | | m/z 366.15; found m/z |
| | | | | | | 366.20. |
| 49 | 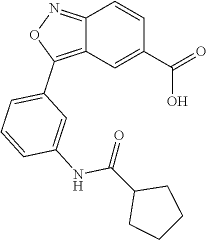 [SMILES: O=C(O)c1ccc2noc(-c3cccc(NC(=O)C4CCCC4)c3)c2c1] | ++ | ++ | | +++ | ¹H NMR (400 MHz, DMSO-d₆) δ 10.25 (s, 1H), 8.64 (s, 1H), 8.45 (s, 1H), 7.85 (dd, J = 14.6, 8.8 Hz, 2H), 7.76 (dd, J = 17.5, 8.6 Hz, 2H), 7.59 (t, J = 8.0 Hz, 1H), 2.82 (p, J = 7.9 Hz, 1H), 1.88 (h, J = 6.3, 5.0 Hz, 2H), 1.81-1.63 (m, 4H), 1.63-1.50 (m, 2H); ¹³C NMR (101 MHz, DMSO-d₆) δ 175.22, 167.02, 166.67, |
| | | | | | | 157.69, 140.71, |
| | | | | | | 130.69, 130.53, |
| | | | | | | 127.85, 127.26, |
| | | | | | | 125.22, 121.92, |
| | | | | | | 121.49, 116.82, |
| | | | | | | 115.40, 113.58, 45.61, |
| | | | | | | 30.23, 25.84; MS |
| | | | | | | (ESI) for [M + H]⁺ |
| | | | | | | (C₂₀H₁₉N₂O₄⁺): calcd. |
| | | | | | | m/z 351.13; found m/z |
| | | | | | | 351.10. |
| 50 | 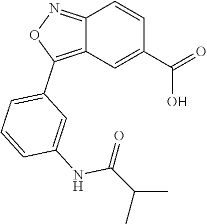 [SMILES: CC(C)C(=O)Nc1cccc(-c2onc3ccc(C(=O)O)cc23)c1] | + | ++ | | ++ | ¹H NMR (400 MHz, DMSO-d₆) δ 10.19 (s, 1H), 8.62 (s, 1H), 8.43 (s, 1H), 7.84 (dd, J = 12.9, 8.8 Hz, 2H), 7.74 (dd, J = 19.2, 8.6 Hz, 2H), 7.58 (t, J = 8.0 Hz, 1H), 2.62 (p, J = 6.8 Hz, 1H), 1.12 (d, J = 6.8 Hz, 6H); ¹³C NMR (101 MHz, DMSO-d₆) δ 176.08, 167.00, 166.67, 157.69, 140.70, |
| | | | | | | 130.70, 130.53, |
| | | | | | | 127.87, 127.26, |
| | | | | | | 125.21, 121.96, |
| | | | | | | 121.53, 116.86, |
| | | | | | | 115.40, 113.58, 35.32, |
| | | | | | | 19.60; MS (ESI) for |
| | | | | | | [M + H]⁺ |
| | | | | | | (C₁₈H₁₇N₂O₄⁺): calcd. |
| | | | | | | m/z 325.12; found m/z |
| | | | | | | 325.10. |
| 51 | 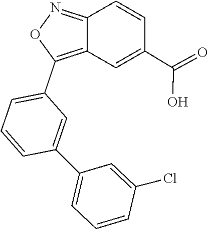 [SMILES: O=C(O)c1ccc2noc(-c3cccc(-c4cccc(Cl)c4)c3)c2c1] | ++ | ++ | | +++ | ¹H NMR (400 MHz, DMSO-d₆) δ 8.64 (s, 1H), 8.31 (s, 1H), 8.14 (d, J = 7.8 Hz, 1H), 7.95 (d, J = 7.8 Hz, 1H), 7.88 (dd, J = 5.6, 3.6 Hz, 2H), 7.82-7.73 (m, 3H), 7.59-7.47 (m, 2H); ¹³C NMR (101 MHz, DMSO-d₆) δ 166.83, 166.53, 157.55, 141.29, 140.12, 133.95, 130.97, 130.60, 130.56, 130.03, |
| | | | | | | 128.03, 127.69, |
| | | | | | | 127.58, 126.85, |
| | | | | | | 126.69, 125.80, |
| | | | | | | 125.35, 125.25, |
| | | | | | | 115.17, 113.78; MS |
| | | | | | | (ESI) for [M + H]⁺ |
| | | | | | | (C₂₀H₁₃NO₃Cl⁺): |
| | | | | | | calcd. m/z 350.06; |
| | | | | | | found m/z 350.00. |
| 52 | 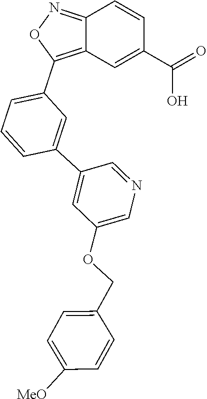 [SMILES: COc1ccc(COc2cncc(-c3cccc(-c4onc5ccc(C(=O)O)cc45)c3)c2)cc1] | ++ | ++ | | ++ | ¹H NMR (400 MHz, DMSO-d₆) δ 8.69 (s, 1H), 8.61 (d, J = 1.8 Hz, 1H), 8.40 (d, J = 2.5 Hz, 2H), 8.22- 8.17 (m, 1H), 8.02 (dd, J = 7.7, 1.7 Hz, 1H), 7.89 (dd, J = 5.7, 3.5 Hz, 2H), 7.85-7.76 (m, 2H), 7.48-7.41 (m, 2H), 7.00-6.94 (m, 2H), 5.23 (s, 2H), 3.76 (s, 3H); MS (ESI) for [M + H]⁺ (C₂₇H₂₁N₂O₅⁺): calcd. m/z 453.15; found m/z 453.10. |
| 53 | 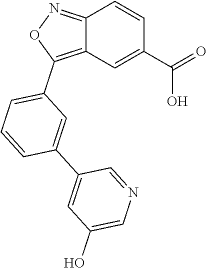 [SMILES: O=C(O)c1ccc2noc(-c3cccc(-c4cncc(O)c4)c3)c2c1] | ++ | ++ | | | ¹H NMR (400 MHz, TFA-d) δ 8.98 (s, 1H), 8.69 (s, 1H), 8.55-8.48 (m, 1H), 8.39 (d, J = 19.3 Hz, 3H), 8.24- 8.16 (m, 1H), 7.92 (d, J = 7.8 Hz, 2H), 7.80 (d, J = 9.5 Hz, 1H); ¹³C NMR (101 MHz, TFA-d) δ 173.27, 170.49, 159.31, 158.99, 144.27, 137.25, 134.72, 134.19, 133.72, 133.55, 133.27, 131.76, 130.76, 130.25, 129.48, |
| | | | | | | 129.13, 128.37, |
| | | | | | | 116.82, 116.58. MS |
| | | | | | | (ESI) for [M + H]⁺ |
| | | | | | | (C₁₉H₁₃N₂O₄⁺): calcd. |
| | | | | | | m/z 333.09; found m/z |
| | | | | | | 333.10. |

### Example 3: 3-(4-Benzylphenyl)benzo[c]isoxazole-5-carboxylic Acid

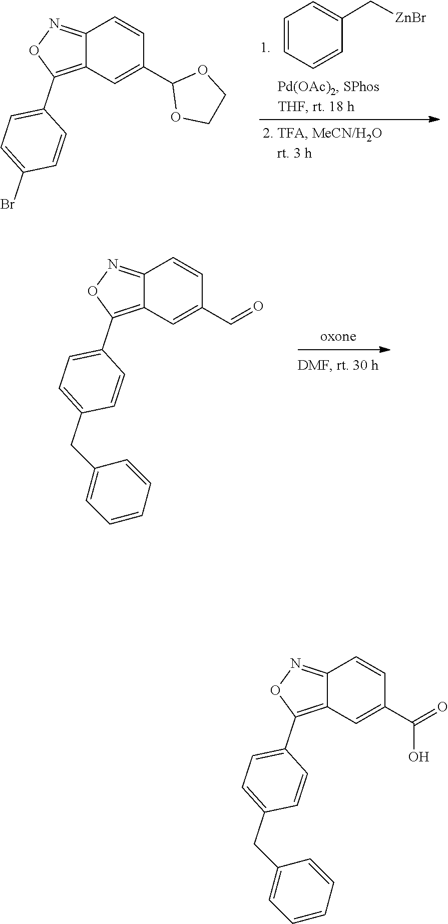

To a mixture of 3-(4-bromophenyl)-5-(1,3-dioxolan-2-yl)benzo[c]isoxazole (69.2 mg, 0.200 mmol), SPhos (3.28 mg, 8.0 μmol) and Pd(OAc)₂ (0.90 mg, 4.0 μmol) in dry THF (0.2 mL, inhibitor free) was added benzylzinc bromide (0.50 mL, 0.5 M in THF, 0.24 μmol) under the nitrogen atmosphere slowly. The reaction mixture was stirred at room temperature for 24 hours, then quenched by sat. NH₄Cl solution and extracted with EtOAc (3×). The organic phase was wash with 10% thiourea aqueous solution and brine, dried (Na₂SO₄) and concentrated under reduced pressure. To a solution of the residue in MeCN (1.2 mL) and water (0.3 mL) was added TFA (76.6 μL, 1.00 mmol). The reaction mixture was stirred at room temperature for 3 hours, neutralized with a saturated NaHCO₃ solution, and extracted with CH₂Cl₂. The organic phase was dried (Na₂SO₄) and concentrated under reduced pressure. The residue was purified by a silica gel column (hexane/EtOAc gradient) to afford 3-(4-benzylphenyl)benzo[c]isoxazole-5-carbaldehyde as a yellow solid (36.4 mg, 0.116 mmol, 58%). ¹H NMR (400 MHz, CDCl₃) δ 10.00 (s, 1H), 8.38 (s, 1H), 8.02 (d, J=8.3 Hz, 2H), 7.84 (dd, J=9.4, 1.3 Hz, 1H), 7.73-7.65 (m, 1H), 7.46 (d, J=8.1 Hz, 2H), 7.36 (dd, J=8.0, 6.6 Hz, 2H), 7.31-7.21 (m, 3H), 4.12 (d, J=1.2 Hz, 2H); MS (ESI) for [M+H]⁺ (C₂₁H₁₆NO₂⁺): calcd. m/z 314.12; found m/z 314.10; LC-MS: 95% purity.

To a solution of 3-(4-benzylphenyl)benzo[c]isoxazole-5-carbaldehyde (36.4 mg, 0.116 mmol) in N,N-dimethylformamide (1.2 mL) was added Oxone triple salt (71.3 mg, 0.116 mmol). The reaction mixture was stirred at room temperature for 18 hours. Upon the completion of the reaction (monitored by LC-MS), the reaction mixture was purified by a reverse phase column to afford 3-(4-benzylphenyl)benzo[c]isoxazole-5-carboxylic acid as a yellow solid (29.1 mg, 88.4 μmol, 76%). ¹H NMR (400 MHz, DMSO-d₆) δ 8.61 (d, J=1.7 Hz, 1H), 8.10-8.00 (m, 2H), 7.86 (d, J=9.3 Hz, 1H), 7.71 (d, J=9.1 Hz, 1H), 7.59-7.46 (m, 2H), 7.36-7.24 (m, 4H), 7.22 (td, J=6.3, 2.6 Hz, 1H), 4.07 (d, J=1.5 Hz, 2H). ¹³C NMR (101 MHz, DMSO-d₆) δ 167.07, 166.59, 157.51, 145.28, 140.49, 130.57, 130.12, 128.88, 128.63, 127.64, 127.17, 126.28, 125.28, 124.74, 115.03, 113.29, 40.96. MS (ESI) for [M+H]⁺ (C₂₁H₁₆NO₃⁺): calcd. m/z 330.11; found m/z 330.10.

Table 3 describes compounds prepared following procedures described in Example 3 using appropriate reagents. (Note: IC₅₀ (determined from enzyme-coupled assays): ++++ means <10 nM; +++ means between 10-100 nM, ++ means between 100 nM-1 μM; + means between 1-100 μM; − means inactive.)

| | | | | | | Physical Data |
|---|---|---|---|---|---|---|
| | | IP6K1 | IP6K2 | IP6K3 | IPMK | MS m/z (M + 1) or and |
| Cmpd | Structure | IC₅₀ | IC₅₀ | IC₅₀ | IC₅₀ | ¹HNMR |
| 54 | 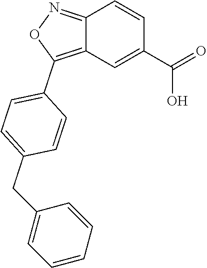 [SMILES: O=C(O)c1ccc2noc(-c3ccc(Cc4ccccc4)cc3)c2c1] | ++ | +++ | + | +++ | ¹H NMR (400 MHz, DMSO-d₆) δ 8.61 (d, J = 1.7 Hz, 1H), 8.10- 8.00 (m, 2H), 7.86 (d, J = 9.3 Hz, 1H), 7.71 (d, J = 9.1 Hz, 1H), 7.59-7.46 (m, 2H), 7.36-7.24 (m, 4H), 7.22 (td, J = 6.3, 2.6 Hz, 1H), 4.07 (d, J = 1.5 Hz, 2H). ¹³C NMR (101 MHz, DMSO-d₆) δ 167.07, 166.59, 157.51, 145.28, 140.49, 130.57, 130.12, 128.88, 128.63, 127.64, |
| | | | | | | 127.17, 126.28, |
| | | | | | | 125.28, 124.74, |
| | | | | | | 115.03, 113.29, 40.96. |
| | | | | | | MS (ESI) for [M + H]⁺ |
| | | | | | | (C₂₁H₁₆NO₃⁺): calcd. |
| | | | | | | m/z 330.11; found m/z |
| | | | | | | 330.10. |
| 55 | 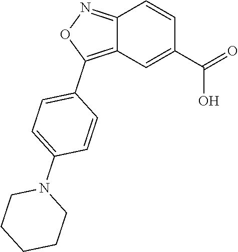 [SMILES: O=C(O)c1ccc2noc(-c3ccc(N4CCCCC4)cc3)c2c1] | ++ | ++ | + | − | ¹H NMR (400 MHz, CDCl₃) δ 8.75 (d, J = 1.4 Hz, 1H), 8.01 (d, J = 8.8 Hz, 2H), 7.89 (dd, J = 9.5, 1.4 Hz, 1H), 7.58 (d, J = 9.5 Hz, 1H), 7.14 (d, J = 8.9 HZ, 2H). 3.42 (t, J = 5.5 Hz, 4H), 1.78- 1.65 (m, 6H). MS (ESI) for [M + H]⁺ (C₁₉H₁₉N₂O₃⁺): calcd. m/z 323.14; found m/z 323.20. |
| 56 | 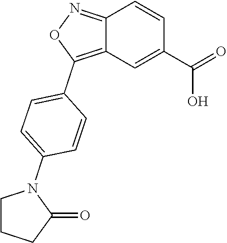 [SMILES: O=C(O)c1ccc2noc(-c3ccc(N4CCCC4=O)cc3)c2c1] | ++ | ++ | + | + | ¹H NMR (400 MHz, DMSO-d₆) δ 8.60 (s, 1H), 8.14 (d, J = 8.9 Hz, 2H), 7.98 (d, J = 8.9 Hz, 2H), 7.89 (d, J = 9.3 Hz, 1H), 7.66 (d, J = 9.3 Hz, 1H), 3.93 (d, J = 7.0 Hz, 2H), 2.57 (t, J = 8.1 Hz, 2H), 2.15-2.07 (m, 2H); MS (ESI) for [M + H]⁺ (C₁₈H₁₅N₂O₄⁺): calcd. m/z 323.10; found m/z 323.10. |
| 57 | 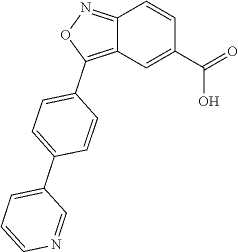 [SMILES: O=C(O)c1ccc2noc(-c3ccc(-c4cccnc4)cc3)c2c1] | +++ | +++ | ++ | +++ | ¹H NMR (400 MHz, DMSO-d₆) δ 9.02 (s, 1H), 8.67 (d, J = 16.6 Hz, 2H), 8.24 (dd, J = 21.2, 8.1 Hz, 3H), 8.06 (d, J = 8.0 Hz, 2H), 7.89 (d, J = 9.4 Hz, 1H), 7.77 (d, J = 9.4 Hz, 1H), 7.56 (dd, J = 8.1, 4.7 Hz, 1H); ¹³C NMR (101 MHz, DMSO-d₆) δ 166.51, 157.59, 149.16, 147.65, 139.73, 134.58, 134.44, 130.57, 128.22, |
| | | | | | | 127.73, 127.68, |
| | | | | | | 127.61, 126.42, |
| | | | | | | 125.27, 124.17, |
| | | | | | | 115.24, 113.74; MS |
| | | | | | | (ESI) for [M + H]⁺ |
| | | | | | | (C₁₉H₁₃N₂O₃⁺): calcd. |
| | | | | | | m/z 317.09; found m/z |
| | | | | | | 317.10. |
| 58 | 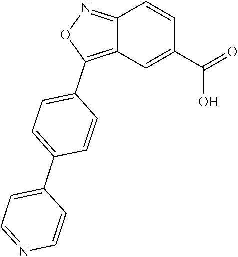 [SMILES: O=C(O)c1ccc2noc(-c3ccc(-c4ccncc4)cc3)c2c1] | ++ | ++ | ++ | +++ | ¹H NMR (400 DMSO-d₆) δ 8.80- 8.61 (m, 3H), 8.29 (d, J = 8.2 Hz, 2H), 8.12 (d, J = 8.1 Hz, 2H), 7.94-7.72 (m, 4H). ¹³C NMR (101 MHz, DMSO-d₆) δ 164.09, 157.80, 152.63, 150.44, 145.77, 139.02, 132.99, 128.13, 127.93, 127.10, 121.34, 121.22, 114.43, 113.48, 60.74, 59.58. MS (ESI) for [M + H]⁺ |
| | | | | | | (C₁₉H₁₃N₂O₃⁺): calcd. |
| | | | | | | m/z 317.09; found m/z |
| | | | | | | 317.10. |

### Example 4: 3-(3-(Piperidin-4-yloxy)phenyl)-5-(1H-tetrazol-5-yl)benzo[c]isoxazole Hydrochloride and 1-(4-(3-(5-(1H-tetrazol-5-yl)benzo[c]isoxazol-3-yl)phenoxy)piperidin-1-yl)ethan-1-one

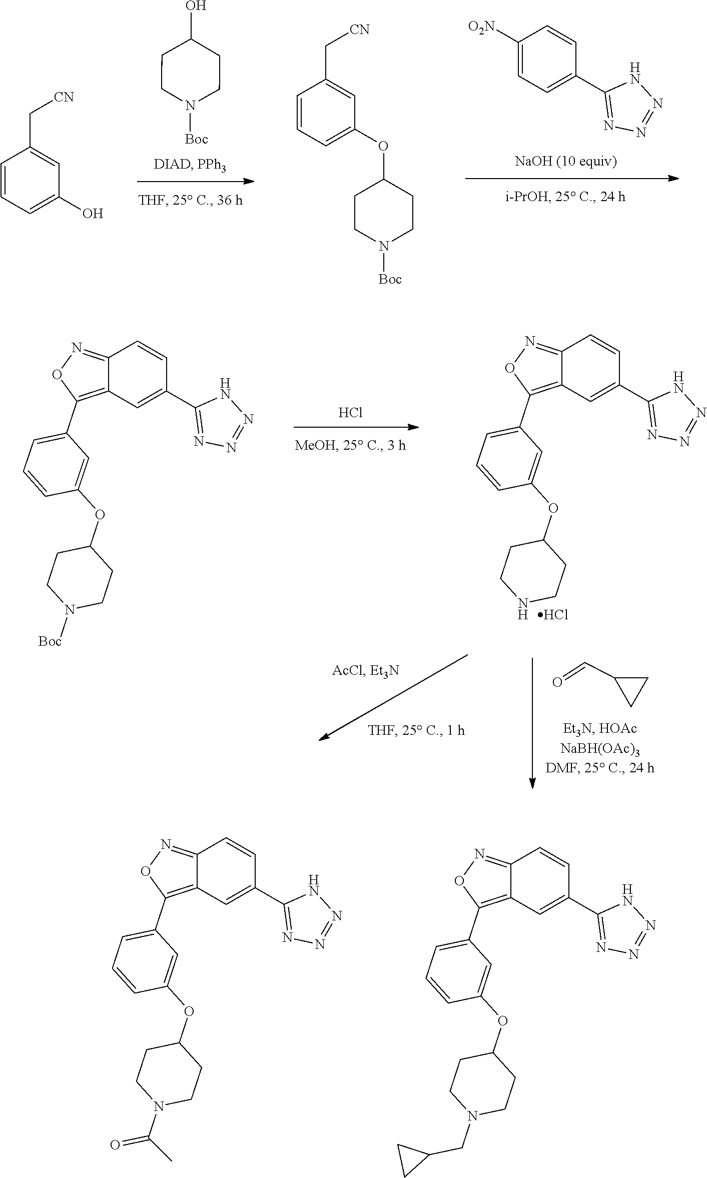

To a solution of 2-(3-hydroxyphenyl)acetonitrile (2.00 g, 15.0 mmol), tert-butyl 4-hydroxypiperidine-1-carboxylate (3.33 g, 16.5 mmol) and PPh₃ (4.33 g, 16.5 mmol) in anhydrous THF (15 mL) was added DIAD (3.28 mL, 16.5 mmol) at 0° C. The resulting mixture was stirred at room temperature for 24 hours. Upon completion, the reaction passed through a short pad of silica gel. The silica gel was then washed with eluent (hexane/EtOAc 2:1, 150 mL). The organic phase was concentrated to about 50 mL and washed with 1 M NaOH aq. for three times and brine once. The organic layer was dried (Na₂SO₄) and concentrated under high vacuum. The residue was dissolved in minimum amount of CH₂Cl₂ and loaded to silica gel column. The product was isolated from silica column chromatography as an oil, which was poured into hexane (45 mL). The mixture was sonicated until a white solid crushed out to afford a fine suspension. The precipitate was filtered and washed by hexane to afford tert-butyl 4-(3-(cyanomethyl)phenoxy)piperidine-1-carboxylate as a white solid (3.82 g, 12.1 mmol, 80%). ¹H NMR (400 MHz, Chloroform-d) δ 7.31-7.26 (m, 1H), 6.94-6.82 (m, 3H), 4.49 (tt, J=7.1, 3.5 Hz, 1H), 3.75-3.65 (m, 2H), 3.72 (s, 2H), 3.35 (ddd, J=13.5, 7.6, 3.8 Hz, 2H), 1.98-1.87 (m, 2H), 1.75 (dtd, J=13.8, 7.3, 3.7 Hz, 2H), 1.47 (s, 9H); MS (ESI) for [M+Na]⁺ (₁₈H₂₄N₂O₃Na⁺): calcd. m/z 339.17; found m/z 339.20; LC-MS: 96% purity.

NaOH pellets were granulated (0.98 g, 24 mmol) and added to 2-PrOH (25 mL). The mixture was stirred for 15 minutes before the addition of 5-(4-nitrophenyl)-1H-tetrazole (703 mg, 3.68 mmol). After the tetrazole was dissolved, tert-butyl 4-(3-(cyanomethyl)phenoxy)piperidine-1-carboxylate (775 mg, 2.45 mmol) was added. The reaction mixture was stirred at room temperature and monitored by LCMS. Upon the completion of reaction, to the suspension was added HOAc to adjust pH to 5. The reaction mixture was poured into brine and extracted by EtOAc for three times. The combined organic phase was dried (Na₂SO₄) and the solvent was removed. The residue was purified by reverse phase chromatography to afford the tert-butyl 4-(3-(5-(1H-tetrazol-5-yl)benzo[c]isoxazol-3-yl)phenoxy)piperidine-1-carboxylate as a yellow solid (310 mg, 0.671 mmol, 27%). ¹H NMR (500 MHz, CD₃OD) δ 8.68 (s, 1H), 8.03-7.96 (m, 1H), 7.77-7.70 (m, 2H), 7.67 (d, J=2.2 Hz, 1H), 7.58 (t, J=8.0 Hz, 1H), 7.25 (dd, J=8.2, 2.5 Hz, 1H), 3.49 (ddd, J=12.8, 9.1, 3.6 Hz, 2H), 3.35-3.27 (m, 2H), 2.27 (ddd, J=12.9, 9.0, 3.7 Hz, 2H), 2.13 (dq, J=11.0, 3.4 Hz, 2H); ¹³C NMR (126 MHz, CD₃OD) δ 166.21, 157.48, 157.27, 130.84, 129.12, 128.71, 121.34, 120.76, 119.74, 118.42, 116.17, 114.01, 113.85, 68.71, 40.52, 26.98 (one sp² carbon is missing); MS (ESI) for [M+H]⁺ (C₁₉H₁₉N₆O₂⁺): calcd. m/z 363.16; found m/z 363.15; LC-MS: 98% purity.

To a solution of tert-butyl 4-(3-(5-(1H-tetrazol-5-yl)benzo[c]isoxazol-3-yl)phenoxy)piperidine-1-carboxylate (310 mg, 0.671 mmol) in MeOH (3.4 mL) was added conc. HCl (0.55 mL, 37%). The resulting solution was stirred at room temperature for 3 hours. The reaction was concentrated under vacuo. The residue was sonicated in minimum amount of 1:1 MeOH/H₂O and collected by centrifuge to afford 3-(3-(piperidin-4-yloxy)phenyl)-5-(1H-tetrazol-5-yl)benzo[c]isoxazole hydrochloride as a yellow solid (229 mg, 0.574 mmol, 86%). ¹H NMR (500 MHz, CD₃OD) δ 8.68 (s, 1H), 8.03-7.96 (m, 1H), 7.77-7.70 (m, 2H), 7.67 (d, J=2.2 Hz, 1H), 7.58 (t, J=8.0 Hz, 1H), 7.25 (dd, J=8.2, 2.5 Hz, 1H), 3.49 (ddd, J=12.8, 9.1, 3.6 Hz, 2H), 3.35-3.27 (m, 2H), 2.27 (ddd, J=12.9, 9.0, 3.7 Hz, 2H), 2.13 (dq, J=11.0, 3.4 Hz, 2H); ¹³C NMR (126 MHz, CD₃OD) δ 166.21, 157.48, 157.27, 130.84, 129.12, 128.71, 121.34, 120.76, 119.74, 118.42, 116.17, 114.01, 113.85, 68.71, 40.52, 26.98 (one sp² carbon is missing); MS (ESI) for [M+H]+(C₁₉H₁₉N₆O₂⁺): calcd. m/z 363.16; found m/z 363.15; LC-MS: 99% purity.

To a solution of 3-(3-(piperidin-4-yloxy)phenyl)-5-(1H-tetrazol-5-yl)benzo[c]isoxazole hydrochloride (30.0 mg, 75.2 μmol), Et₃N (11 μL, 75 μmol), and HOAc (6.5 μL, 0.113 mmol) in DMF (0.75 mL) was added cyclopropanecarboxaldehyde (11 μL, 0.150 mmol). The resulting mixture was stirred at room temperature for 20 minutes before the addition of NaBH(OAc)₃ (31.9 mg, 0.150 mmol). The reaction was stirred at room temperature for 24 hours before it was quenched by MeOH. The solvent was evaporated under reduced pressure and the residue was subjected to preparative HPLC to afford 3-(3-((1-(cyclopropylmethyl)piperidin-4-yl)oxy)phenyl)-5-(1H-tetrazol-5-yl)benzo[c]isoxazole as a yellow solid (19.4 mg, 46.6 μmol, 62%). ¹H NMR (400 MHz, CD₃OD) δ 8.76-8.69 (m, 1H), 8.01 (dd, J=9.4, 1.4 Hz, 1H), 7.81-7.65 (m, 3H), 7.60 (td, J=8.0, 5.8 Hz, 1H), 7.28 (ddd, J=14.3, 8.2, 2.5 Hz, 1H), 5.06-4.97 (m, 1H), 3.81 (d, J=12.6 Hz, 1H), 3.67-3.59 (m, 1H), 3.51-3.24 (m, 2H), 3.14 (dd, J=11.2, 7.4 Hz, 2H), 2.55-1.99 (m, 4H), 1.26-0.15 (m, 1H), 0.88-0.73 (m, 2H), 0.49 (dd, J=4.8, 1.5 Hz, 2H); ¹³C NMR (101 MHz, CD₃OD) δ 167.71, 159.06, 158.77, 158.75, 132.30, 130.54, 130.21, 122.76, 122.28, 121.29, 119.86, 117.66, 115.81, 115.23, 71.87, 68.30, 63.02, 62.11, 51.61, 29.71, 28.08, 6.55, 4.95; MS (ESI) for [M+H]⁺ (C₂₃H₂₅N₆O₂⁺): calcd. m/z 417.20; found m/z 417.20; LC-MS: 97% purity.

To a solution of 3-(3-(piperidin-4-yloxy)phenyl)-5-(1H-tetrazol-5-yl)benzo[c]isoxazole hydrochloride (44.2 mg, 0.122 mmol) and Et₃N (34 μL, 0.24 mmol) in THF (0.5 mL) was added AcCl (8.7 μL, 0.12 mmol) in drowse at 0° C. The reaction was stirred at room temperature for 1 hour. Upon the completion, the reaction was quenched by MeOH. The solvent was evaporated under vacuo and the residue was washed in 1:1 MeOH/H₂O. The precipitate was collected by centrifuge to afford 1-(4-(3-(5-(1H-tetrazol-5-yl)benzo[c]isoxazol-3-yl)phenoxy)piperidin-1-yl)ethan-1-one as a yellow solid (24.6 mg, 60.8 μmol, 50%). ¹H NMR (500 MHz, DMSO-d₆) δ 8.72 (d, J=1.5 Hz, 1H), 8.06 (dd, J=9.4, 1.4 Hz, 1H), 7.91 (d, J=9.4 Hz, 1H), 7.74 (dd, J=7.7, 1.6 Hz, 1H), 7.67-7.61 (m, 2H), 7.31 (dd, J=8.3, 2.5 Hz, 1H), 4.83 (tt, J=7.9, 3.7 Hz, 1H), 3.92 (dt, J=13.2, 5.1 Hz, 1H), 3.77-3.70 (m, 1H), 3.26 (td, J=9.3, 4.6 Hz, 1H), 2.03 (s, 4H), 2.01-1.94 (m, 1H), 1.68 (dp, J=13.2, 4.7, 4.3 Hz, 1H), 1.57 (ddt, J=13.2, 8.8, 4.3 Hz, 1H) (one proton is overlapping with DMSO moisture); ¹³C NMR (126 MHz, DMSO-d₆) δ 168.17, 165.94, 164.02, 157.71, 156.97, 131.53, 131.17, 129.61, 128.17, 121.05, 119.40, 118.86, 116.55, 114.00, 113.67, 72.38, 42.94, 38.06, 30.92, 30.19, 21.29; MS (ESI) for [M+H]⁺ (C₂₁H₂₁N₆O₃⁺): calcd. m/z 405.17; found m/z 405.20; LC-MS: 98% purity.

Table 4 describes compounds prepared following procedures described in Example 4 using appropriate reagents. (Note: IC₅₀ (determined from enzyme-coupled assays): ++++ means <10 nM; +++ means between 10-100 nM, ++ means between 100 nM-1 μM; + means between 1-100 μM; − means inactive.)

| | | | | | | Physical Data |
|---|---|---|---|---|---|---|
| | | IP6K1 | IP6K2 | IP6K3 | IPMK | MS m/z (M + 1) or and |
| Cmpd | Structure | IC₅₀ | IC₅₀ | IC₅₀ | IC₅₀ | ¹HNMR |
| 59 | 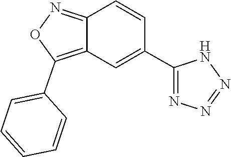 [SMILES: c1ccc(-c2onc3ccc(-c4nnn[nH]4)cc23)cc1] | +++ | +++ | ++ | +++ | ¹H NMR (400 MHz, DMSO-d₆) δ 8.76 (t, J = 1.1 Hz, 1H), 8.20-8.12 (m, 2H), 8.06 (dd, J = 9.3, 1.5 Hz, 1H), 7.90 (dd, J = 9.3, 1.0 Hz, 1H), 7.77-7.61 (m, 3H); ¹³C NMR (101 MHz, DMSO-d₆) δ 166.08, 156.96, 154.95, 131.43, |
| | | | | | | 129.77, 129.59, 126.90, |
| | | | | | | 126.76, 121.00, 120.97, |
| | | | | | | 116.51, 113.56; MS |
| | | | | | | (ESI) for [M + H]⁺ |
| | | | | | | (C₁₄H₁₀N₅O⁺): calcd. |
| | | | | | | m/z 264.09; found m/z |
| | | | | | | 264.10. |
| 60 | 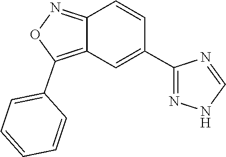 [SMILES: c1ccc(-c2onc3ccc(-c4nc[nH]n4)cc23)cc1] | + | ++ | + | +++ | ¹H NMR (400 MHz, DMSO-d₆) δ 8.61 (t, J = 1.2 Hz, 1H), 8.53 (s, 1H), 8.17-8.07 (m, 3H), 7.81 (dd, J = 9.3, 1.0 Hz, 1H), 7.76-7.57 (m, 3H). ¹³C NMR (101 MHz, DMSO-d₆) δ 164.88, 157.16, 146.81, 130.99, 130.00, 129.77, |
| | | | | | | 129.04, 128.12, 127.27, |
| | | | | | | 126.53, 117.48, 115.77, |
| | | | | | | 113.87. MS (ESI) for |
| | | | | | | [M + H]⁺ (C₁₅H₁₁N₄O⁺): |
| | | | | | | calcd. m/z 263.09; |
| | | | | | | found m/z 263.10. |
| 61 | 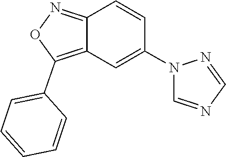 [SMILES: c1ccc(-c2onc3ccc(-n4cncn4)cc23)cc1] | + | + | + | +++ | ¹H NMR (400 MHz, DMSO-d₆) δ 9.46 (s, 1H), 8.48-8.43 (m, 1H), 8.30 (s, 1H), 8.17 (dt, J = 6.2, 1.4 Hz, 2H), 8.00 (dd, J = 9.6, 1.8 Hz, 1H), 7.92 (dd, J = 9.6, 1.0 Hz, 1H), 7.72-7.58 (m, 3H). ¹³C NMR (101 MHz, DMSO-d₆) δ |
| | | | | | | 165.34, 156.30, 152.53, |
| | | | | | | 143.08, 133.46, 131.04, |
| | | | | | | 129.67, 127.00, 126.62, |
| | | | | | | 126.05, 117.28, 112.99, |
| | | | | | | 109.89. MS (ESI) for |
| | | | | | | [M + H]⁺ (C₁₅H₁₁N₄O⁺): |
| | | | | | | calcd. m/z 263.09; |
| | | | | | | found m/z 263.10. |
| 62 | 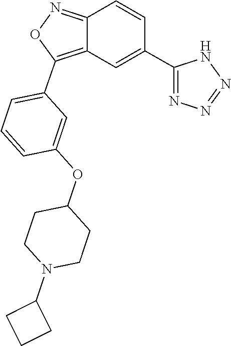 [SMILES: c1cc(OC2CCN(C3CCC3)CC2)cc(-c2onc3ccc(-c4nnn[nH]4)cc23)c1] | +++ | ++ | | +++ | ¹H NMR (400 MHz, CD₃OD) δ 8.77-8.68 (m, 1H), 8.01 (dd, J = 9.4, 1.5 Hz, 1H), 7.81- 7.65 (m, 3H), 7.60 (td, J = 8.0, 6.7 Hz, 1H), 7.27 (ddd, J = 14.8, 8.4, 2.5 Hz, 1H), 4.99 (t, J = 3.0 Hz, 1H), 3.81 (dt, J = 29.7, 8.3 Hz, 1H), 3.67- 3.38 (m, 2H), 3.30-3.01 (m, 2H), 2.54-2.26 (m, 6H), 2.24-1.83 (m, 4H); ¹³C NMR (101 MHz, CD₃OD) δ 167.66, 159.03, 158.72, 157.75, 132.28, 130.51, 130.18, 122.67, 122.29, 121.28, 119.83, 117.64, 115.83, 115.28, 71.93, 68.13, 60.46, 48.85, 45.78, 29.50, 27.78, 26.80, |
| | | | | | | 14.38; MS (ESI) for |
| | | | | | | [M + H]⁺ (C₂₃H₂₅N₆O₂⁺): |
| | | | | | | calcd. m/z 417.20; |
| | | | | | | found m/z 417.20. |
| 63 | 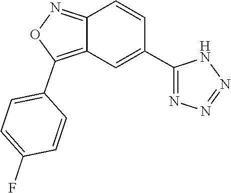 [SMILES: Fc1ccc(-c2onc3ccc(-c4nnn[nH]4)cc23)cc1] | +++ | +++ | ++ | +++ | ¹H NMR (400 MHz, DMSO-d₆) δ 8.74 (d, J = 1.3 Hz, 1H), 8.29- 8.17 (m, 2H), 8.05 (dd, J = 9.4, 1.4 Hz, 1H), 7.90 (dd, J = 9.4, 1.0 Hz, 1H). ¹³C NMR (101 MHz, DMSO-d₆) δ 165.23, 164.94, 162.44, 156.96, 129.61, 129.41 (d, J = 9.0 Hz), 123.60 (d, J = 3.4 Hz), 120.99, |
| | | | | | | 117.13, 116.91, 116.48, |
| | | | | | | 113.37. MS (ESI) for |
| | | | | | | [M + H]⁺ (C₁₄H₉FN₅O⁺): |
| | | | | | | calcd. m/z 282.08; |
| | | | | | | found m/z 282.10. |
| 64 | 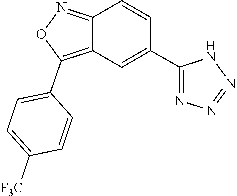 [SMILES: FC(F)(F)c1ccc(-c2onc3ccc(-c4nnn[nH]4)cc23)cc1] | +++ | +++ | ++ | +++ | ¹H NMR (400 MHz, DMSO-d₆) δ 8.78 (d, J = 1.3 Hz, 1H), 8.36 (d, J = 8.2 Hz, 2H), 8.11- 7.99 (m, 3H), 7.96 (dd, J = 9.3, 1.0 Hz, 1H). ¹³C NMR (101 MHz, DMSO-d₆) δ 164.30, 157.05, 130.90, 130.58, 130.38, 129.76, 127.52, 126.58 (q, J = 3.9 Hz), 123.80 (q, J = 274 Hz), |
| | | | | | | 120.54, 116.79, 114.49. |
| | | | | | | MS (ESI) for [M + H]⁺ |
| | | | | | | (C₁₅H₉F₃N₅O⁺): calcd. |
| | | | | | | m/z 332.08; found m/z |
| | | | | | | 332.10. |
| 65 | 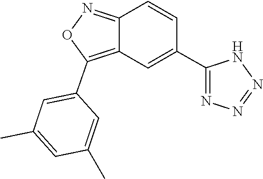 [SMILES: Cc1cc(C)cc(-c2onc3ccc(-c4nnn[nH]4)cc23)c1] | +++ | +++ | ++ | +++ | ¹H NMR (400 MHz, DMSO-d₆) δ 8.72 (t, J = 1.3 Hz, 1H), 8.05 (dd, J = 9.3, 1.4 Hz, 1H), 7.89 (dd, J = 9.3, 1.0 Hz, 1H), 7.76 (d, J = 1.6 Hz, 2H), 7.29 (d, J = 1.8 Hz, 1H), 2.44 (s, 6H). ¹³C NMR (101 MHz, DMSO-d₆) δ 166.39, 156.93, 154.82, 139.12, |
| | | | | | | 132.94, 129.59, 126.82, |
| | | | | | | 124.32, 121.15, 120.77, |
| | | | | | | 116.47, 113.40, 20.89. |
| | | | | | | MS (ESI) for [M + H]⁺ |
| | | | | | | (C₁₆H₁₄N₅O⁺): calcd. |
| | | | | | | m/z 292.12; found m/z |
| | | | | | | 292.10. |
| 66 | 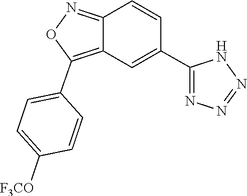 [SMILES: FC(F)(F)Oc1ccc(-c2onc3ccc(-c4nnn[nH]4)cc23)cc1] | +++ | +++ | ++ | +++ | ¹H NMR (400 MHz, DMSO-d₆) δ 8.75 (d, J = 1.3 Hz, 1H), 8.34- 8.25 (m, 2H), 8.08 (dd, J = 9.3, 1.4 Hz, 1H), 7.94 (dd, J = 9.4, 1.0 Hz, 1H), 7.76-7.66 (m, 2H). ¹³C NMR (101 MHz, DMSO-d₆) δ 164.66, 157.01, 155.24, 150.00 (q, J = 1.7 Hz), 129.80, 129.07, 126.04, 122.18, 121.69, 120.56, |
| | | | | | | 120.01 (q, J = 259 Hz), |
| | | | | | | 116.55, 113.92. MS |
| | | | | | | (ESI) for [M + H]⁺ |
| | | | | | | (C₁₅H₉F₃N₅O₂)⁺: calcd. |
| | | | | | | m/z 348.07; found m/z |
| | | | | | | 348.05. |
| 67 | 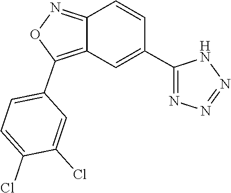 [SMILES: Clc1ccc(-c2onc3ccc(-c4nnn[nH]4)cc23)cc1Cl] | +++ | ++ | ++ | +++ | ¹H NMR (400 MHz, DMSO-d₆) δ 8.76 (s, 1H), 8.34 (d, J = 2.1 Hz, 1H), 8.14 (dd, J = 8.5, 2.1 Hz, 1H), 8.07 (d, J = 9.3 Hz, 1H), 7.92 (dd, J = 8.9, 5.2 Hz, 2H). ¹³C NMR (101 MHz, DMSO-d₆) δ 163.51, 157.02, 154.91, 134.00, 132.70, 131.94, 129.82, 128.18, 127.10, 126.79, |
| | | | | | | 121.65, 120.63, 116.65, |
| | | | | | | 114.15. MS (ESI) for |
| | | | | | | [M + H]⁺ |
| | | | | | | (C₁₄H₈Cl₂N₅O⁺): calcd. |
| | | | | | | m/z 332.01; found m/z |
| | | | | | | 332.00. |
| 68 | 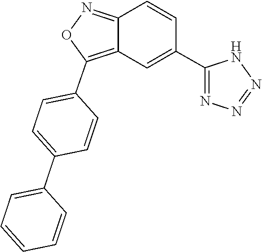 [SMILES: c1ccc(-c2ccc(-c3onc4ccc(-c5nnn[nH]5)cc34)cc2)cc1] | +++ | +++ | ++ | ++++ | ¹H NMR (400 MHz, DMSO-d₆) δ 8.81 (d, J = 1.3 Hz, 1H), 8.32- 8.18 (m, 2H), 8.07 (dd, J = 9.3, 1.4 Hz, 1H), 7.99 (d, J = 8.4 Hz, 2H), 7.91 (dd, J = 9.5, 1.0 Hz, 1H), 7.84-7.76 (m, 2H), 7.54 (t, J = 7.5 Hz, 2H), 7.50-7.41 (m, 1H). ¹³C NMR (101 MHz, DMSO-d₆) δ 165.74, 157.01, 155.04, 142.74, 138.75, 129.64, 129.18, 128.42, 127.85, 127.30, 126.90, 125.83, 121.05, |
| | | | | | | 120.99, 116.52, 113.67. |
| | | | | | | MS (ESI) for [M + H]⁺ |
| | | | | | | (C₂₀H₁₄N₅O⁺): calcd. |
| | | | | | | m/z 340.12; found m/z |
| | | | | | | 340.10. |
| 69 | 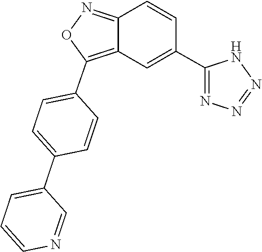 [SMILES: c1cncc(-c2ccc(-c3onc4ccc(-c5nnn[nH]5)cc34)cc2)c1] | +++ | +++ | ++ | ++++ | ¹H NMR (400 MHz, DMSO-d₆) δ 9.03 (s, 1H), 8.63 (m, 2H), 8.28- 8.17 (m, 4H), 8.11-8.02 (m, 2H), 7.78 (dd, J = 9.4, 0.8 Hz, 1H), 7.56 (dd, J = 8.0, 4.7 Hz, 1H). ¹³C NMR (101 MHz, DMSO-d₆) δ 172.01, 163.32, 157.21, 149.16, 147.76, 138.93, 134.47, 134.29, 131.50, 128.10, 127.99, 127.09, 126.97, 124.04, 115.64, 115.35, 114.52. MS (ESI) for [M + H]⁺ |
| | | | | | | (C₁₉H₁₃N₆O⁺): calcd. |
| | | | | | | m/z 341.12; found m/z |
| | | | | | | 341.10. |
| 70 | 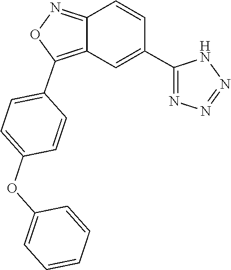 [SMILES: c1ccc(Oc2ccc(-c3onc4ccc(-c5nnn[nH]5)cc34)cc2)cc1] | + | ++ | ++ | ++++ | ¹H NMR (400 MHz, DMSO-d₆) 8.44 (s, 1H), 8.18 (dd, J = 9.3, 1.3 Hz, 1H), 8.15-8.06 (m, 2H), 7.72-7.65 (m, 1H), 7.52-7.44 (m, 2H), 7.26 (dd, J = 8.4, 2.1 Hz, 3H), 7.18 (dd, J = 7.7, 1.5 Hz, 2H). MS (ESI) for [M + H]⁺ (C₂₀H₁₄N₅O₂⁺): calcd. m/z 356.11; found m/z 356.10. |
| 71 | 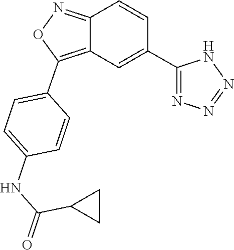 [SMILES: O=C(Nc1ccc(-c2onc3ccc(-c4nnn[nH]4)cc23)cc1)C1CC1] | +++ | ++ | ++++ | ++++ | ¹H NMR (400 MHz, DMSO-d₆) δ 10.63 (s, 1H), 8.77 (s, 1H), 8.18- 8.09 (m, 2H), 8.04 (dd, J = 9.3, 1.4 Hz, 1H), 7.96-7.89 (m, 2H), 7.87 (d, J = 9.4 Hz, 1H), 1.89-1.80 (m, 1H), 0.93-0.82 (m, 4H). ¹³C NMR (126 MHz, DMSO-d₆) δ 172.35, 166.09, 156.94, 142.14, 133.51, 129.51, 127.66, 121.45, 121.25, 119.44, 116.34, 112.93, 40.02, 14.83, 7.68. MS (ESI) |
| | | | | | | for [M + H]⁺ |
| | | | | | | (C₁₈H₁₅N₆O₂⁺): calcd. |
| | | | | | | m/z 347.13; found m/z |
| | | | | | | 347.10. |
| 72 | 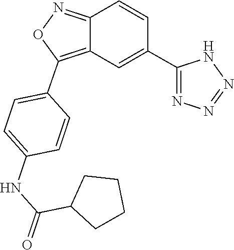 [SMILES: O=C(Nc1ccc(-c2onc3ccc(-c4nnn[nH]4)cc23)cc1)C1CCCC1] | +++ | +++ | ++++ | +++ | ¹H NMR (400 MHz, DMSO-d₆) δ 10.30 (s, 1H), 8.76 (s, 1H), 8.12 (d, J = 8.7 Hz, 2H), 8.03 (d, J = 9.3 Hz, 1H), 7.94 (d, J = 8.7 Hz, 2H), 7.86 (d, J = 9.4 Hz, 1H), 2.84 (p, J = 7.8 Hz, 1H), 1.95-1.82 (m, 2H), 1.82-1.62 (m, 4H), 1.63-1.51 (m, 2H). ¹³C NMR (101 MHz, DMSO-d₆) δ 175.15, 166.14, 156.99, 142.35, 129.56, 127.63, 125.41, 121.46, 121.29, 120.34, |
| | | | | | | 119.61, 116.38, 112.97, |
| | | | | | | 45.53, 30.12, 25.75. MS |
| | | | | | | (ESI) for [M +H]⁺ |
| | | | | | | (C₂₀H₁₉N₆O₂⁺): calcd. |
| | | | | | | m/z 375.16; found m/z |
| | | | | | | 375.20. |
| 73 | 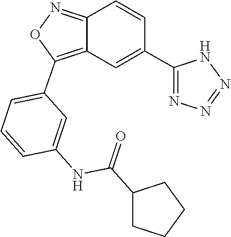 [SMILES: O=C(Nc1cccc(-c2onc3ccc(-c4nnn[nH]4)cc23)c1)C1CCCC1] | +++ | +++ | +++ | ++++ | ¹H NMR (400 MHz, DMSO-d₆) δ 10.25 (s, 1H), 8.73 (s, 1H), 8.45 (s, 1H), 8.06 (d, J = 9.3 Hz, 1H), 7.92 (d, J = 9.4 Hz, 1H), 7.85 (t, J = 7.8 Hz, 2H), 7.63 (t, J = 8.0 Hz, 1H), 2.83 (p, J = 7.9 Hz, 1H), 1.95-1.82 (m, 2H), 1.82-1.63 (m, 4H), 1.64-1.50 (m, 2H); ¹³C NMR (214 MHz, DMSO-d₆) δ 175.14, 166.10, 163.72, 157.13, 140.72, 130.40, 129.85, |
| | | | | | | 127.33, 121.83, 121.32, |
| | | | | | | 120.76, 116.83, 116.68, |
| | | | | | | 114.41, 113.74, 45.53, |
| | | | | | | 30.19, 25.80; MS (ESI) |
| | | | | | | for [M + H]⁺ |
| | | | | | | (C₂₀H₁₉N₆O₂⁺): calcd. |
| | | | | | | m/z 375.16; found m/z |
| | | | | | | 375.10. |
| 74 | 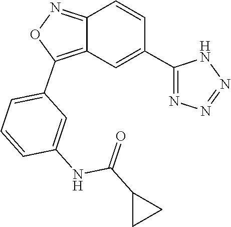 [SMILES: O=C(Nc1cccc(-c2onc3ccc(-c4nnn[nH]4)cc23)c1)C1CC1] | +++ | +++ | +++ | ++++ | ¹H NMR (400 MHz, DMSO-d₆) δ 10.57 (s, 1H), 8.72 (s, 1H), 8.42 (s, 1H), 8.04 (d, J = 9.3 Hz, 1H), 7.90 (d, J = 9.4 Hz, 1H), 7.83 (d, J = 8.1 Hz, 2H), 7.62 (t, J = 8.0 Hz, 1H), 1.83 (t, J = 6.2 Hz, 1H), 0.91-0.84 (m, 4H); ¹³C NMR (214 MHz, DMSO-d₆) δ 172.37, 166.10, 157.09, 155.41, 140.55, 130.41, 129.74, 127.33, 121.72, |
| | | | | | | 121.33, 121.30, 120.87, |
| | | | | | | 116.73, 116.68, 113.71, |
| | | | | | | 14.83, 7.59; MS (ESI) |
| | | | | | | for [M + H]⁺ |
| | | | | | | (C₁₈H₁₅N₆O₂⁺): calcd. |
| | | | | | | m/z 347.13; found m/z |
| | | | | | | 347.10. |
| 75 | 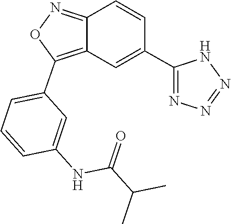 [SMILES: CC(C)C(=O)Nc1cccc(-c2onc3ccc(-c4nnn[nH]4)cc23)c1] | ++ | ++ | ++ | ++++ | ¹H NMR (400 MHz, DMSO-d⁶) δ 10.00 (s, 1H), 7.91 (s, 1H), 7.70 (s, 1H), 7.48 (d, J = 8.2 Hz, 1H), 7.41 (t, J = 7.9 Hz, 1H), 7.34 (d, J = 7.6 Hz, 1H), 7.21 (t, J = 8.0 Hz, 1H), 7.10-7.03 (m, 1H), 2.60-2.53 (m, 1H), 1.06 (d, J = 5.3 Hz, 6H); ¹³C NMR (214 MHz, DMSO-d⁶) δ 175.71, 175.51, 153.00, 143.99, 139.98, 139.94, 135.78, |
| | | | | | | 134.65, 129.26, 129.10, |
| | | | | | | 127.35, 124.93, 122.52, |
| | | | | | | 121.30, 120.20, 120.09, |
| | | | | | | 118.60, 35.05, 19.58, |
| | | | | | | 19.55 (additional peaks |
| | | | | | | due to amide rotamers); |
| | | | | | | MS (ESI) for [M + H]⁺ |
| | | | | | | (C₁₈H₁₇N₆O₂⁺): calcd. |
| | | | | | | m/z 349.14; found m/z |
| | | | | | | 349.10. |
| 76 | 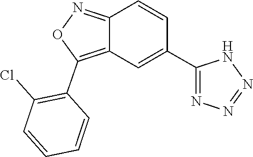 [SMILES: Clc1ccccc1-c1onc2ccc(-c3nnn[nH]3)cc12] | +++ | +++ | ++ | ++++ | ¹H NMR (400 MHz, CD₃OD) δ 8.42 (t, J = 1.3 Hz, 1H), 8.09 (dd, J = 9.4, 1.5 Hz, 1H), 7.89- 7.76 (m, 2H), 7.73 (dd, J = 7.9, 1.5 Hz, 1H), 7.62 (dtd, J = 18.0, 7.5, 1.7 Hz, 2H); ¹³C NMR (101 MHz, CD₃OD) δ 166.90, 158.26, 158.07, |
| | | | | | | 134.08, 133.74, 132.95, |
| | | | | | | 132.12, 130.83, 128.86, |
| | | | | | | 127.67, 123.22, 121.99, |
| | | | | | | 117.53, 117.37; MS |
| | | | | | | (ESI) for [M + H]⁺ |
| | | | | | | (C₁₄H₉N₅ClO₁⁺): calcd. |
| | | | | | | m/z 298.05; found m/z |
| | | | | | | 298.10. |
| 77 | 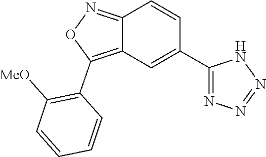 [SMILES: COc1ccccc1-c1onc2ccc(-c3nnn[nH]3)cc12] | +++ | +++ | ++ | +++ | ¹H NMR (850 MHz, CD₃OD) δ 8.59 (s, 1H), 8.04 (d, J = 9.2 Hz, 1H), 7.84 (d, J = 8.0 Hz, 1H), 7.72 (d, J = 9.2 Hz, 1H), 7.60 (t, J = 7.6 Hz, 1H), 7.30 (d, J = 8.4 Hz, 1H), 7.19 (t, J = 7.4 Hz, 1H), 4.00 (s, 3H); ¹³C NMR (214 MHz, CD₃OD) δ |
| | | | | | | 166.95, 158.59, 158.09, |
| | | | | | | 134.08, 132.74, 131.31, |
| | | | | | | 130.99, 123.34, 122.90, |
| | | | | | | 122.26, 117.82, 116.81, |
| | | | | | | 116.73, 113.19, 56.36; |
| | | | | | | MS (ESI) for [M + H]⁺ |
| | | | | | | (C₁₅H₁₂N₅O₂⁺): calcd. |
| | | | | | | m/z 294.10; found m/z |
| | | | | | | 294.10. |
| 78 | 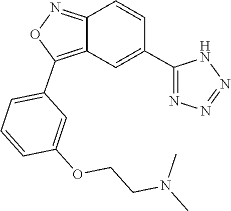 [SMILES: CN(C)CCOc1cccc(-c2onc3ccc(-c4nnn[nH]4)cc23)c1] | ++ | ++ | ++ | ++++ | ¹H NMR (400 MHz, DMSO-d₆) δ 10.48 (s, 1H), 9.13 (s, 1H), 8.15 (dd, J = 9.4, 1.4 Hz, 1H), 7.96-7.87 (m, 2H), 7.83 (t, J = 2.1 Hz, 1H), 7.64 (t, J = 8.0 Hz, 1H), 7.30 (dd, J = 8.1, 2.5 Hz, 1H), 4.60 (t, J = 5.1 Hz, 2H), 3.59 (t, J = 5.0 Hz, 2H), 2.89 (s, 6H); ¹³C NMR (214 MHz, DMSO-d₆) δ 165.84, |
| | | | | | | 158.29, 157.77, 157.02, |
| | | | | | | 131.11, 129.77, 127.94, |
| | | | | | | 124.59, 121.57, 120.08, |
| | | | | | | 117.98, 116.51, 113.68, |
| | | | | | | 112.98, 62.58, 55.26, |
| | | | | | | 42.75; MS (ESI) for |
| | | | | | | [M + H]⁺ (C₁₈H₁₉N₆O₂⁺): |
| | | | | | | calcd. m/z 351.16; |
| | | | | | | found m/z 351.20. |
| 79 | 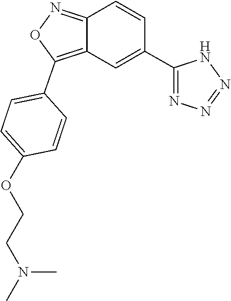 | ++ | ++ | ++ | + | ¹H NMR (850 MHz, DMSO-d₆) δ 10.68 (s, 1H), 9.08 (s, 1H), 8.27 (d, J = 8.4 Hz, 2H), 8.09 (dd, J = 9.2, 1.4 Hz, 1H), 7.85 (d, J = 9.2 Hz, 1H), 7.30 (d, J = 8.4 Hz, 2H), 4.53 (t, J = 5.0 Hz, 2H), 3.57 (t, J = 5.0 Hz, 2H), 2.87 (s, 6H); ¹³C NMR (214 MHz, DMSO-d₆) δ 166.11, 159.87, 156.95, 129.58, 128.78, 128.32, 124.59, 121.81, 120.18, 116.20, 115.94, 112.77, 62.63, 55.14, 42.76; MS (ESI) for [M + H]⁺ (C₁₈H₁₉N₆O₂⁺): calcd. |
| | | | | | | m/z 351.16; found m/z |
| | | | | | | 351.20. |
| 80 | 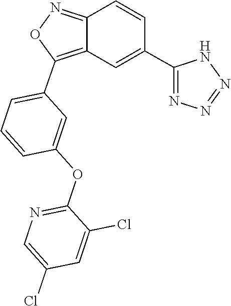 [SMILES: Clc1cnc(Oc2cccc(-c3onc4ccc(-c5nnn[nH]5)cc34)c2)c(Cl)c1] | +++ | +++ | +++ | +++ | ¹H NMR (400 MHz, DMSO-d₆) δ 8.77 (s, 1H), 8.39 (d, J = 2.3 Hz, 1H), 8.21 (d, J = 2.3 Hz, 1H), 8.07 (dd, J = 9.3, 1.5 Hz, 2H), 8.00 (t, J = 2.0 Hz, 1H), 7.93 (d, J = 9.4 Hz, 1H), 7.77 (t, J = 8.0 Hz, 1H), 7.49 (dd, J = 8.1, 2.4 Hz, 1H); ¹³C NMR (101 MHz, DMSO-d₆) δ 165.13, 157.01, 156.92, 154.89, 153.90, 143.77, 139.49, 131.38, 129.73, 128.27, 125.63, 124.40, 123.77, 121.24, 120.94, 119.57, 118.79, 116.60, 113.87; MS (ESI) for [M +H]⁺ |
| | | | | | | (C₁₉H₁₁Cl₂N₆O₂⁺): |
| | | | | | | calcd. m/z 425.03; |
| | | | | | | found m/z 425.00. |
| 81 | 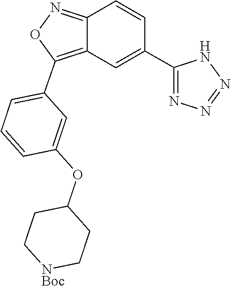 [SMILES: CC(C)(C)OC(=O)N1CCC(Oc2cccc(-c3onc4ccc(-c5nnn[nH]5)cc34)c2)CC1] | +++ | +++ | +++ | ++++ | ¹H NMR (400 MHz, CD₃OD) δ 8.72 (d, J = 1.6 Hz, 1H), 8.09 (d, J = 9.3 Hz, 1H), 7.80-7.72 (m, 2H), 7.67 (q, J = 1.9 Hz, 1H), 7.58 (t, J = 8.0 Hz, 1H), 7.23 (dd, J = 8.3, 2.5 Hz, 1H), 3.78 (ddd, J = 12.0, 7.1, 3.9 Hz, 2H), 3.41 (d, J = 10.7 Hz, 2H), 2.03 (dd, J = 13.0, 7.6 Hz, 2H), 1.76 (dtd, J = 12.0, 7.8, 3.8 Hz, 2H), 1.48 (s, 9H), (one proton is overlapping with CD₃OD peak); MS (ESI) for [M + H]⁺ |
| | | | | | | (C₂₄H₂₇N₆O₄⁺): calcd. |
| | | | | | | m/z 463.21; found m/z |
| | | | | | | 463.15. |
| 82 | 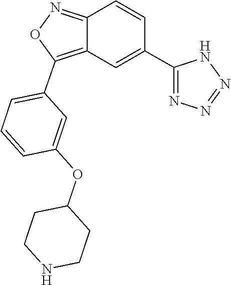 [SMILES: c1cc(OC2CCNCC2)cc(-c2onc3ccc(-c4nnn[nH]4)cc23)c1] | +++ | +++ | ++ | ++++ | ¹H NMR (500 MHz, CD₃OD) δ 8.68 (s, 1H), 8.03-7.96 (m, 1H), 7.77-7.70 (m, 2H), 7.67 (d, J = 2.2 Hz, 1H), 7.58 (t, J = 8.0 Hz, 1H), 7.25 (dd, J = 8.2, 2.5 Hz, 1H), 3.49 (ddd, J = 12.8, 9.1, 3.6 Hz, 2H), 3.35-3.27 (m, 2H), 2.27 (ddd, J = 12.9, 9.0, 3.7 Hz, 2H), 2.13 (dq, J = 11.0, 3.4 Hz, 2H), (one proton is overlapping with CD₃OD peak); ¹³C NMR (126 MHz, CD₃OD) δ 166.21, 157.48, 157.27, 130.84, |
| | | | | | | 129.12, 128.71, 121.34, |
| | | | | | | 120.76, 119.74, 118.42, |
| | | | | | | 116.17, 114.01, 113.85, |
| | | | | | | 68.71, 40.52, 26.98 |
| | | | | | | (one sp² carbon is |
| | | | | | | missing); MS (ESI) for |
| | | | | | | [M + H]⁺ (C₁₉H₁₉N₆O₂⁺): |
| | | | | | | calcd. m/z 363.16; |
| | | | | | | found m/z 363.15. |
| 83 | 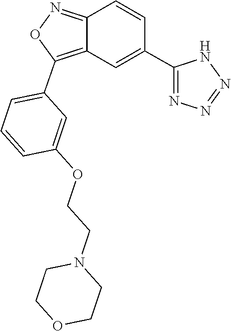 [SMILES: c1cc(OCCN2CCOCC2)cc(-c2onc3ccc(-c4nnn[nH]4)cc23)c1] | +++ | ++ | ++ | +++ | ¹H NMR (500 MHz, DMSO-d₆) δ 9.79 (s, 1H), 8.94 (dd, J = 9.4, 1.5 Hz, 1H), 8.75 (d, J = 9.4 Hz, 1H), 8.70 (dd, J = 7.6, 1.6 Hz, 1H), 8.61 (t, J = 2.1 Hz, 1H), 8.49 (t, J = 8.0 Hz, 1H), 8.14 (dd, J = 8.3, 2.5 Hz, 1H), 5.45 (t, J = 4.9 Hz, 2H), 4.91-4.67 (m, 4H), 4.46 (t, J = 4.9 Hz, 2H) (four protons overlap with DMSO moister peak); ¹³C NMR (126 MHz, DMSO-d₆) δ 165.79, 158.28, 157.02, 131.15, 129.73, 128.01, 121.26, 121.00, 120.04, 119.11, 117.94, 116.55, |
| | | | | | | 113.72, 112.92, 63.21, |
| | | | | | | 62.46, 55.00, 51.76; MS |
| | | | | | | (ESI) for [M + H]⁺ |
| | | | | | | (C₂₀H₂₁N₆O₃⁺): calcd. |
| | | | | | | m/z 393.17; found m/z |
| | | | | | | 393.20. |
| 84 | 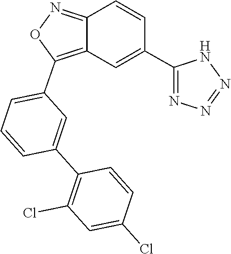 [SMILES: Clc1ccc(-c2cccc(-c3onc4ccc(-c5nnn[nH]5)cc34)c2)c(Cl)c1] | +++ | +++ | | +++ | ¹H NMR (400 MHz, DMSO-d₆) δ 8.73 (s, 1H), 8.25-8.18 (m, 1H), 8.14 (d, J = 1.8 Hz, 1H), 8.05 (dd, J = 9.3, 1.4 Hz, 1H), 7.90 (d, J = 9.5 Hz, 1H), 7.85-7.77 (m, 2H), 7.70 (dt, J = 7.8, 1.4 Hz, 1H), 7.63-7.54 (m, 2H); ¹³C NMR (126 MHz, DMSO-d₆) δ 165.59, 156.99, 155.02, 138.95, 137.59, 133.58, 132.87, 132.46, 132.18, 129.87, 129.68, 129.42, 127.86, 127.22, 126.98, |
| | | | | | | 126.48, 121.23, 120.80, |
| | | | | | | 116.55, 113.80; MS |
| | | | | | | (ESI) for [M + H]⁺ |
| | | | | | | (C₂₀H₁₂Cl₂N₅O⁺): |
| | | | | | | calcd. m/z 408.04; |
| | | | | | | found m/z 408.05. |
| 85 | 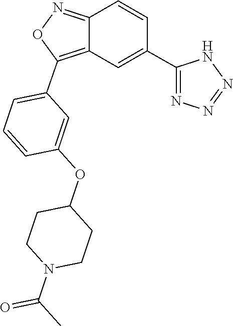 [SMILES: CC(=O)N1CCC(Oc2cccc(-c3onc4ccc(-c5nnn[nH]5)cc34)c2)CC1] | ++++ | +++ | | +++ | ¹H NMR (500 MHz, DMSO-d₆) δ 8.72 (d, J = 1.5 Hz, 1H), 8.06 (dd, J = 9.4, 1.4 Hz, 1H), 7.91 (d, J = 9.4 Hz, 1H), 7.74 (dd, J = 7.7, 1.6 Hz, 1H), 7.67-7.61 (m, 2H), 7.31 (dd, J = 8.3, 2.5 Hz, 1H), 4.83 (tt, J = 7.9, 3.7 Hz, 1H), 3.92 (dt, J = 13.2, 5.1 Hz, 1H), 3.77-3.70 (m, 1H), 3.26 (td, J = 9.3, 4.6 Hz, 1H), 2.03 (s, 4H), 2.01- 1.94 (m, 1H), 1.68 (dp, J = 13.2, 4.7, 4.3 Hz, 1H), 1.57 (ddt, J = 13.2, 8.8, 4.3 Hz, 1H) (one proton is overlapping with DMSO moisture); |
| | | | | | | ¹³C NMR (126 MHz, |
| | | | | | | DMSO-d₆) δ 168.17, |
| | | | | | | 165.94, 164.02, 157.71, |
| | | | | | | 156.97, 131.53, 131.17, |
| | | | | | | 129.61, 128.17, 121.05, |
| | | | | | | 119.40, 118.86, 116.55, |
| | | | | | | 114.00, 113.67, 72.38, |
| | | | | | | 42.94, 38.06, 30.92, |
| | | | | | | 30.19, 21.29; MS (ESI) |
| | | | | | | for [M + H]⁺ |
| | | | | | | (C₂₁H₂₁N₆O₃⁺): calcd. |
| | | | | | | m/z 405.17; found m/z |
| | | | | | | 405.20. |
| 86 | 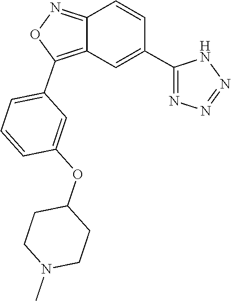 [SMILES: CN1CCC(Oc2cccc(-c3onc4ccc(-c5nnn[nH]5)cc34)c2)CC1] | +++ | +++ | | +++ | ¹H NMR (400 MHz, CD₃OD) δ 8.80-8.74 (m, 1H), 8.04 (dd, J = 9.4, 1.4 Hz, 1H), 7.85- 7.69 (m, 3H), 7.63 (t, J = 7.7 Hz, 1H), 7.34- 7.25 (m, 1H), 4.99 (s, 1H), 3.74-3.61 (d, J = 14.5 Hz, 1H), 3.55-3.41 (m, 2H), 3.37-3.22 (m, 1H), 2.96 (d, J = 6.3 Hz, 3H), 2.55-2.44 (d, J = 14.7 Hz, 1H), 2.39-2.29 (d, J = 14.5 Hz, 1H), 2.26-2.11 (m, 1H), 2.07-1.92 (m, 1H); MS (ESI) for [M + H]⁺ (C₂₀H₂₁N₆O₂⁺): calcd. m/z 377.17; found m/z |
| | | | | | | 377.20. |
| 87 | 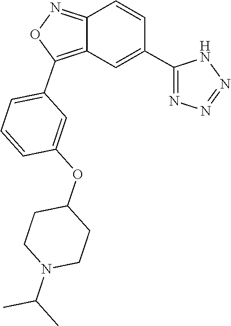 [SMILES: CC(C)N1CCC(Oc2cccc(-c3onc4ccc(-c5nnn[nH]5)cc34)c2)CC1] | +++ | +++ | | +++ | ¹H NMR (500 MHz, CD₃OD) δ 8.68 (dt, J = 10.4, 1.3 Hz, 1H), 7.98 (dt, J = 9.4, 1.3 Hz, 1H), 7.77-7.63 (m, 3H), 7.58 (dt, J = 9.9, 8.0 Hz, 1H), 7.25 (ddd, J = 16.7, 8.1, 2.5 Hz, 1H), 4.98 (t, J = 2.9 Hz, 1H), 3.66-3.56 (m, 2H), 3.48-3.43 (m, 2H), 3.38-3.32 (m, 1H), 2.51 (d, J = 13.9 Hz, 1H), 2.37 (dt, J = 16.0, 2.8 Hz, 1H), 2.33-2.26 (m, 1H), 2.12-1.99 (m, 1H), 1.44 (dd, J = 10.8, 6.7 Hz, 6H); ¹³C NMR (126 MHz, CD₃OD) δ 159.07, 158.76, 158.69, 132.27, 130.48, 122.59, |
| | | | | | | 122.29, 121.22, 120.51, |
| | | | | | | 119.93, 119.83, 117.60, |
| | | | | | | 115.67, 115.25, 68.37, |
| | | | | | | 45.27, 30.14, 28.29, |
| | | | | | | 17.15; MS (ESI) for |
| | | | | | | [M + H]⁺ (C₂₂H₂₅N₆O₂⁺): |
| | | | | | | calcd. m/z 405.20; |
| | | | | | | found m/z 405.20. |
| 88 | 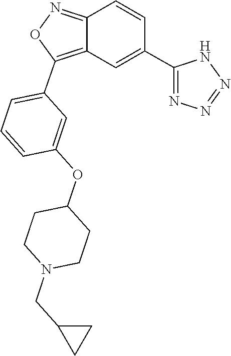 [SMILES: c1cc(OC2CCN(CC3CC3)CC2)cc(-c2onc3ccc(-c4nnn[nH]4)cc23)c1] | +++ | ++ | | +++ | ¹H NMR (400 MHz, CD₃OD) δ 8.76-8.69 (m, 1H), 8.01 (dd, J = 9.4, 1.4 Hz, 1H), 7.81- 7.65 (m, 3H), 7.60 (td, J = 8.0, 5.8 Hz, 1H), 7.28 (ddd, J = 14.3, 8.2, 2.5 Hz, 1H), 5.06-4.97 (m, 1H), 3.81 (d, J = 12.6 Hz, 1H), 3.67-3.59 (m, 1H), 3.51-3.24 (m, 2H), 3.14 (dd, J = 11.2, 7.4 Hz, 2H), 2.55-1.99 (m, 4H), 1.26-1.15 (m, 1H), 0.88-0.73 (m, 2H), 0.49 (dd, J = 4.8, 1.5 Hz, 2H); ¹³C NMR 101 MHz, CD₃OD) δ 167.71, 159.06, 158.77, 158.75, 132.30, 130.54, 130.21, 122.76, 122.28, 121.29, 119.86, 117.66, |
| | | | | | | 115.81, 115.23, 71.87, |
| | | | | | | 68.30, 63.02, 62.11, |
| | | | | | | 51.61, 29.71, 28.08, |
| | | | | | | 6.55, 4.95; MS (ESI) for |
| | | | | | | [M + H]⁺ (C₂₃H₂₅N₆O₂⁺): |
| | | | | | | calcd. m/z 417.20; |
| | | | | | | found m/z 417.20. |
| 89 | 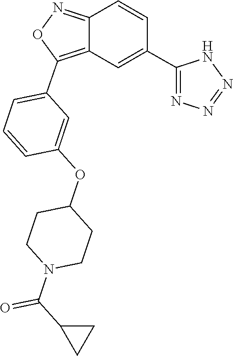 [SMILES: O=C(C1CC1)N1CCC(Oc2cccc(-c3onc4ccc(-c5nnn[nH]5)cc34)c2)CC1] | +++ | +++ | | ++++ | ¹H NMR (500 MHz, DMSO-d₆) δ 8.72 (s, 1H), 8.07 (d, J = 9.3 Hz, 1H), 7.91 (d, J = 9.3 Hz, 1H), 7.74 (d, J = 7.7 Hz, 1H), 7.67 (d, J = 2.5 Hz, 1H), 7.63 (t, J = 8.0 Hz, 1H), 7.32 (dd, J = 8.3, 2.5 Hz, 1H), 4.85 (tt, J = 8.0, 3.8 Hz, 1H), 4.09- 3.89 (m, 2H), 3.66-3.44 (m, 2H), 2.14-1.93 (m, 3H), 1.63 (d, J = 53.7 Hz, 2H), 0.78-0.65 (m, 4H); ¹³C NMR (101 MHz, DMSO-d₆) δ 171.03, 165.92, 157.72, 156.97, 154.91, 131.18, 129.62, 128.18, 120.99, 119.40, 118.86, 116.54, 114.01, 113.68, 72.51, 42.11, 31.22, 30.24, |
| | | | | | | 10.37, 6.90 (one sp² |
| | | | | | | carbon is missing and |
| | | | | | | one sp³ overlaps with |
| | | | | | | DMSO-d₆ septet); MS |
| | | | | | | (ESI) for [M + H]⁺ |
| | | | | | | (C₂₃H₂₃N₆O₃⁺): calcd. |
| | | | | | | m/z 431.18; found m/z |
| | | | | | | 431.20. |
| 90 | 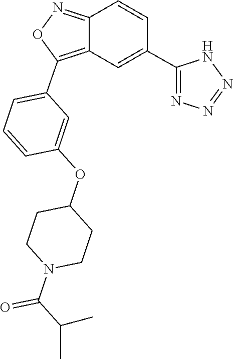 [SMILES: CC(C)C(=O)N1CCC(Oc2cccc(-c3onc4ccc(-c5nnn[nH]5)cc34)c2)CC1] | +++ | +++ | | ++++ | ¹H NMR (400 MHz, DMSO-d₆) δ 8.70 (s, 1H), 8.06 (dd, J = 9.4, 1.3 Hz, 1H), 7.90 (d, J = 9.4 Hz, 1H), 7.73 (d, J = 7.7 Hz, 1H), 7.69-7.58 (m, 2H), 7.30 (dd, J = 8.3, 2.5 Hz, 1H), 4.83 (dt, J = 8.1, 4.1 Hz, 1H), 3.88 (dd, J = 51.6, 12.7 Hz, 2H), 3.62-3.56 (m, 2H), 2.90 (p, J = 6.7 Hz, 1H), 2.10-1.92 (m, 2H), 1.72-1.52 (m, 2H), 1.01 (d, J = 6.7 Hz, 6H) (two protons partially overlap with DMSO-d₆ moisture peak); ¹³C NMR (101 MHz, DMSO-d₆) δ 174.31, 165.93, 157.71, 156.98, 154.99, 131.19, 129.63, |
| | | | | | | 128.19, 121.01, 119.41, |
| | | | | | | 118.87, 116.56, 114.01, |
| | | | | | | 113.69, 72.46, 41.98, |
| | | | | | | 38.42, 31.32, 30.34, |
| | | | | | | 29.04, 19.50 (one sp² |
| | | | | | | carbon is missing); MS |
| | | | | | | (ESI) for [M + H]⁺ |
| | | | | | | (C₂₃H₂₅N₆O₃⁺): calcd. |
| | | | | | | m/z 433.20; found m/z |
| | | | | | | 433.20. |
| 91 | 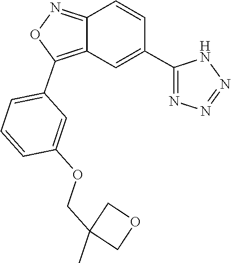 [SMILES: CC1(COc2cccc(-c3onc4ccc(-c5nnn[nH]5)cc34)c2)COC1] | +++ | +++ | | | ¹H NMR (400 MHz, DMSO-d₆) δ 8.70 (t, J = 1.3 Hz, 1H), 8.05 (dd, J = 9.4, 1.4 Hz, 1H), 7.89 (dd, J = 9.4, 1.0 Hz, 1H), 7.77-7.72 (m, 1H), 7.66-7.59 (m, 2H), 7.27 (dd, J = 8.1, 2.5 Hz, 1H), 4.55 (d, J = 5.8 Hz, 2H), 4.35 (d, J = 5.8 Hz, 2H), 4.22 (s, 2H), 1.41 (s, 3H); ¹³C NMR (101 MHz, DMSO-d₆) δ 165.96, 159.60, 157.02, 154.98, 131.13, 129.66, 128.08, 121.04, 120.98, |
| | | | | | | 119.45, 117.99, 116.59, |
| | | | | | | 113.71, 112.39, 78.66, |
| | | | | | | 72.92, 21.04, 17.03; MS |
| | | | | | | (ESI) for [M + H]⁺ |
| | | | | | | (C₁₉H₁₈N₅O₃⁺): calcd. |
| | | | | | | m/z 364.14; found m/z |
| | | | | | | 364.15. |
| 92 | 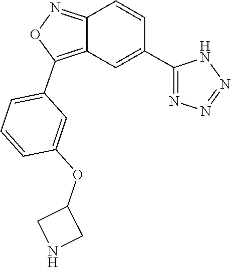 [SMILES: c1cc(OC2CNC2)cc(-c2onc3ccc(-c4nnn[nH]4)cc23)c1] | +++ | +++ | | | ¹H NMR (400 MHz, DMSO-d₆) δ 9.34 (s, 2H), 8.84 (s, 1H), 8.11 (d, J = 9.4 Hz, 1H), 7.88 (dd, J = 12.2, 8.6 Hz, 1H), 7.64 (t, J = 8.0 Hz, 1H), 7.55 (d, J = 1.8 Hz, 1H), 7.16 (dd, J = 8.3, 2.5 Hz, 1H), 5.34 (p, J = 5.6 Hz, 1H), 4.51 (dd, J = 12.0, 6.5 Hz, 2H), 4.08 (dd, J = 12.1, 4.7 Hz, 2H); ¹³C NMR (101 MHz, dmso) δ 165.43, 157.05, 156.58, 155.20, 131.52, 129.94, 128.38, 121.72, 120.70, 120.62, |
| | | | | | | 117.89, 116.47, 113.91, |
| | | | | | | 112.96, 67.76, 52.29; |
| | | | | | | MS (ESI) for [M + H]⁺ |
| | | | | | | (C₁₇H₁₅N₆O₂⁺): calcd. |
| | | | | | | m/z 335.13; found m/z |
| | | | | | | 335.10. |
| 93 | 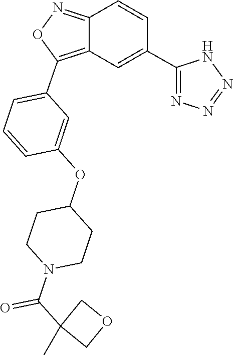 [SMILES: CC1(C(=O)N2CCC(Oc3cccc(-c4onc5ccc(-c6nnn[nH]6)cc45)c3)CC2)COC1] | ++ | +++ | | | ¹H NMR (400 MHz, DMSO-d₆) δ 8.72 (s, 1H), 8.07 (d, J = 9.3 Hz, 1H), 7.92 (d, J = 9.3 Hz, 1H), 7.75 (d, J = 7.7 Hz, 1H), 7.69-7.60 (m, 2H), 7.31 (dd, J = 8.2, 2.5 Hz, 1H), 4.82 (dd, J = 20.3, 4.8 Hz, 3H), 4.27 (d, J = 5.8 Hz, 2H), 3.89 (dt, J = 11.7, 4.6 Hz, 1H), 3.09-3.01 (m, 2H), 2.08-1.95 (m, 2H), 1.72-1.58 (m, 2H), 1.55 (s, 3H) (two protons overlap with DMSO moister peak); ¹³C NMR (101 MHz, DMSO-d₆) δ 172.26, 165.99, 157.68, 157.04, 131.27, 129.73, 128.23, 121.05, 119.54, 118.99, |
| | | | | | | 116.61, 114.09, 113.75, |
| | | | | | | 78.62, 78.50, 72.13, |
| | | | | | | 44.04, 42.07, 38.40, |
| | | | | | | 30.75, 30.04, 22.91 |
| | | | | | | (two sp² carbon is |
| | | | | | | missing); MS (ESI) for |
| | | | | | | [M + H]⁺ (C₂₄H₂₅N₆O₄⁺): |
| | | | | | | calcd. m/z 461.19; |
| | | | | | | found m/z 461.20. |
| 94 | 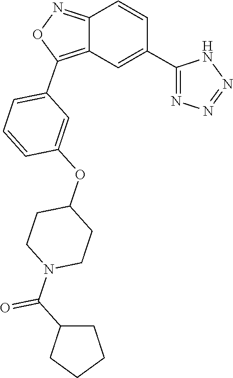 [SMILES: O=C(C1CCCC1)N1CCC(Oc2cccc(-c3onc4ccc(-c5nnn[nH]5)cc34)c2)CC1] | +++ | +++ | | | ¹H NMR (400 MHz, DMSO-d₆) δ 8.71 (s, 1H), 8.06 (d, J = 9.3 Hz, 1H), 7.91 (d, J = 9.4 Hz, 1H), 7.74 (d, J = 7.7 Hz, 1H), 7.68-7.59 (m, 2H), 7.30 (dd, J = 8.4, 2.5 Hz, 1H), 4.83 (dt, J = 8.1, 4.1 Hz, 1H), 3.98- 3.78 (m, 2H), 3.00 (p, J = 7.9 Hz, 2H), 2.09- 1.92 (m, 2H), 1.84-1.45 (m, 11H); ¹³C NMR (101 MHz, DMSO-d₆) δ 173.44, 165.95, 157.76, 157.03, 151.20, 131.24, 129.73, 128.23, 121.16, 120.95, 119.44, 118.90, 116.58, 114.05, 113.74, 72.54, 42.09, 31.29, 30.38, 29.70, 25.73; MS (ESI) for [M + H]⁺ (C₂₅H₂₇N₆O₃⁺): |
| | | | | | | calcd. m/z 459.21; |
| | | | | | | found m/z 459.20. |
| 95 | 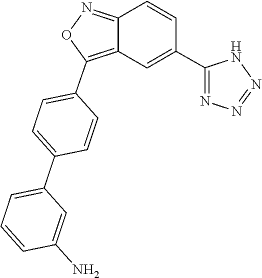 [SMILES: Nc1cccc(-c2ccc(-c3onc4ccc(-c5nnn[nH]5)cc34)cc2)c1] | +++ | +++ | | | ¹H NMR (400 MHz, DMSO-d₆) δ 8.98 (s, 1H), 8.38-8.29 (m, 2H), 8.09 (dd, J = 9.3, 1.4 Hz, 1H), 7.92 (dd, J = 16.1, 8.9 Hz, 3H), 7.74- 7.64 (m, 2H), 7.58 (t, J = 7.8 Hz, 1H), 7.32 (dd, J = 7.8, 2.2 Hz, 1H); ¹³C NMR (101 MHz, DMSO-d₆) δ 165.67, 157.08, 154.83, 141.62, 140.19, 135.66, 130.61, 129.70, 127.89, 127.60, 126.30, 124.58, 121.61, 121.38, 120.97, 119.94, 116.60, 113.80; MS |
| | | | | | | (ESI) for [M + H]⁺ |
| | | | | | | (C₂₀H₁₅N₆O⁺): calcd. |
| | | | | | | m/z 355.13; found m/z |
| | | | | | | 355.10. |
| 96 | 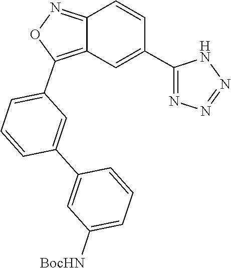 [SMILES: CC(C)(C)OC(=O)Nc1cccc(-c2cccc(-c3onc4ccc(-c5nnn[nH]5)cc34)c2)c1] | +++ | +++ | | | ¹H NMR (400 MHz, DMSO-d₆) δ 9.47 (s, 1H), 8.78 (s, 1H), 8.26 (d, J = 1.9 Hz, 1H), 8.18 (dt, J = 7.7, 1.6 Hz, 1H), 8.09 (dd, J = 9.4, 1.4 Hz, 1H), 7.98-7.85 (m, 3H), 7.82 (t, J = 7.7 Hz, 1H), 7.56-7.49 (m, 1H), 7.46-7.39 (m, 2H), 1.49 (s, 9H); MS (ESI) for [M + H]⁺ (C₂₅H₂₃N₆O₃⁺): calcd. m/z 455.18; found m/z 455.15. |
| 97 | 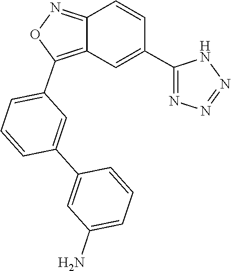 [SMILES: Nc1cccc(-c2cccc(-c3onc4ccc(-c5nnn[nH]5)cc34)c2)c1] | +++ | +++ | | | ¹H NMR (400 MHz, DMSO-d₆) δ 8.86 (s, 1H), 8.31 (s, 1H), 8.24 (d, J = 7.8 Hz, 1H), 8.11 (d, J = 9.4 Hz, 1H), 7.98-7.89 (m, 2H), 7.83 (t, J = 7.8 Hz, 1H), 7.67- 7.59 (m, 2H), 7.54 (t, J = 7.8 Hz, 1H), 7.23 (d, J = 7.9 Hz, 1H); ¹³C NMR (101 MHz, DMSO-d₆) δ 166.05, 157.12, 140.96, 140.39, 130.70, 130.48, 129.83, 129.76, 127.71, 126.44, 124.82, 123.43, 121.20, 121.08, 120.34, 119.15, |
| | | | | | | 116.68, 113.87 (Two |
| | | | | | | sp² carbons are |
| | | | | | | missing); MS MS (ESI) |
| | | | | | | for [M + H]⁺ |
| | | | | | | (C₂₀H₁₅N₆O⁺): calcd. |
| | | | | | | m/z 355.13; found m/z |
| | | | | | | 355.10. |
| 98 | 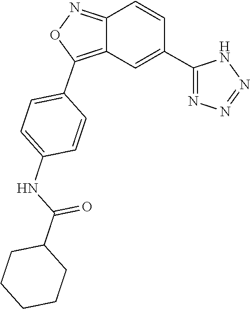 [SMILES: O=C(Nc1ccc(-c2onc3ccc(-c4nnn[nH]4)cc23)cc1)C1CCCCC1] | ++ | | | | ¹H NMR (400 MHz, DMSO-d₆) δ 10.24 (s, 1H), 8.76 (s, 1H), 8.11 (d, J = 8.7 Hz, 2H), 8.03 (d, J = 9.3 Hz, 1H), 7.93 (d, J = 8.6 Hz, 2H), 7.86 (d, J = 9.4 Hz, 1H), 2.39 (tt, J = 11.5, 3.5 Hz, 1H), 1.80 (ddd, J = 27.0, 11.2, 3.8 Hz, 4H), 1.70-1.62 (m, 1H), 1.43 (qd, J = 12.2, 3.0 Hz, 2H), 1.35-1.16 (m, 3H); ¹³C NMR (101 MHz, DMSO-d₆) δ 175.13, 166.16, 157.02, 142.41, 129.62, 127.64, 121.42, 121.33, 120.46, 119.64, 116.39, 113.01, |
| | | | | | | 45.10, 29.16, 25.45, |
| | | | | | | 25.28 (one sp² carbon is |
| | | | | | | missing ; MS (ESI) for |
| | | | | | | [M + H]⁺ (C₂₁H₂₁N₆O₂⁺): |
| | | | | | | calcd. m/z 389.17; |
| | | | | | | found m/z 389.15. |
| 99 | 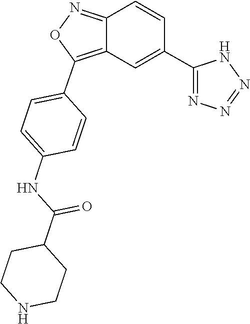 [SMILES: O=C(Nc1ccc(-c2onc3ccc(-c4nnn[nH]4)cc23)cc1)C1CCNCC1] | ++ | | | | ¹H NMR (400 MHz, DMSO-d₆) δ 10.59 (s, 1H), 8.89 (s, 1H), 8.86 (br, 1H), 8.58 (br, 1H), 81.7 (d, J = 8.6 Hz, 2H), 8.06 (d, J = 9.3 Hz, 1H), 7.93 (d, J = 8.6 Hz, 2H), 7.84 (d, J = 9.3 Hz, 1H), 3.41-3.31 (m, 2H), 3.06-2.84 (m, 2H), 2.79-2.69 (m, 1H), 2.02 (dd, J = 14.3, 3.7 Hz, 2H), 1.92-1.77 (m, 2H); ¹³C NMR (101 MHz, DMSO-d₆) δ 172.87, 165.97, 157.04, 155.08, 141.94, 129.72, 127.70, 121.66, 121.36, 120.76, 119.84, 116.32, 113.07, 42.54, 40.08, 25.13; MS |
| | | | | | | (ESI) for [M + H]⁺ |
| | | | | | | (C₂₀H₂₀N₇O₂⁺): calcd. |
| | | | | | | m/z 390.17; found m/z |
| | | | | | | 390.20. |
| 100 | 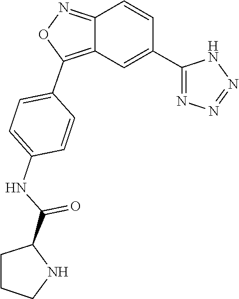 [SMILES: O=C(Nc1ccc(-c2onc3ccc(-c4nnn[nH]4)cc23)cc1)[C@@H]1CCCN1] | ++ | | | | ¹H NMR (400 MHz, DMSO-d₆) δ 11.14 (s, 1H), 9.58 (s, 1H), 8.89 (s, 1H), 8.75 (s, 1H), 8.22 (d, J = 8.6 Hz, 2H), 8.05 (d, J = 9.6 Hz, 1H), 7.93 (d, J = 8.6 Hz, 2H), 7.86 (d, J = 9.4 Hz, 1H), 4.42 (t, J = 7.6, 7.6 Hz, 1H), 2.42 (dd, J = 13.4, 6.7 Hz, 1H), 2.08-1.90 (m, 3H); ¹³C NMR (101 MHz, DMSO-d₆) δ 167.53, 165.83, 157.06, 140.90, 129.70, 127.91, 122.48, 121.47, 120.72, 120.22, 116.47, 113.25, 60.01, 45.97, 29.69, |
| | | | | | | 23.68; [α]_{D}²⁰ = −20.1°, |
| | | | | | | (c 1.0, DMSO); MS |
| | | | | | | (ESI) for [M + H]⁺ |
| | | | | | | (C₁₉H₁₈N₇O₂⁺): calcd. |
| | | | | | | m/z 376.15; found m/z |
| | | | | | | 376.20. |
| 101 | 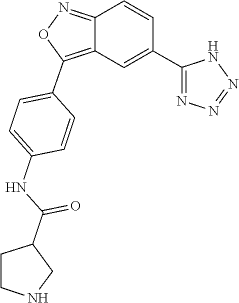 [SMILES: O=C(Nc1ccc(-c2onc3ccc(-c4nnn[nH]4)cc23)cc1)C1CCNC1] | ++ | | | | ¹H NMR (400 MHz, DMSO-d₆) δ 10.78 (s, 1H), 9.15 (s, 2H), 8.90 (s, 1H), 8.19 (d, J = 8.7 Hz, 2H), 8.06 (d, J = 9.3 Hz, 1H), 7.94 (d, J = 8.7 Hz, 2H), 7.85 (d, J = 9.4 Hz, 1H), 2.31 (dq, J = 14.0, 7.2 Hz, 1H, 2.10 (dq, J = 13.5, 7.2 Hz, 1H) (Five protons are overlapping with DMSO moisture); ¹³C NMR (101 MHz, DMSO-d₆) δ 171.11, 166.01, 157.05, 154.88, 141.71, 129.65, 127.78, 121.90, 121.61, 120.48, 119.97, 116.43, 113.11, |
| | | | | | | 46.95, 45.06, 43.32, |
| | | | | | | 29.01; MS (ESI) for |
| | | | | | | [M + H]⁺ (C₁₉H₁₈N₇O₂⁺): |
| | | | | | | calcd. m/z 376.15; |
| | | | | | | found m/z 376.15. |
| 104 | 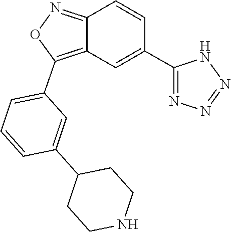 [SMILES: c1cc(-c2onc3ccc(-c4nnn[nH]4)cc23)cc(C2CCNCC2)c1] | ++ | | | | ¹H NMR (400 MHz, DMSO-d₆) δ 8.44 (s, 1H), 8.18 (d, J = 8.9 Hz, 1H), 7.96 (d, J = 8.2 Hz, 1H), 7.90 (s, 1H), 7.73- 7.62 (m, 2H), 7.46 (d, J = 7.8 Hz, 1H), 4.14- 4.01 (m, 1H), 3.17- 2.98 (m, 4H), 2.06 (d, J = 13.5 Hz, 2H), 1.96- 1.80 (m, 2H); ¹³C NMR (101 MHz, DMSO-d₆) δ 167.04, 155.54, 149.20, 130.67, 129.02, |
| | | | | | | 128.23, 126.20, 122.48, |
| | | | | | | 122.45, 117.46, 114.44, |
| | | | | | | 110.52, 44.53, 39.89, |
| | | | | | | 30.09 MS (ESI) for |
| | | | | | | [M + H]⁺ (C₁₉H₁₉N₆O⁺): |
| | | | | | | calcd. m/z 347.16; |
| | | | | | | found m/z 347.40. |
| 105 | 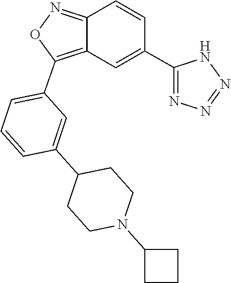 [SMILES: c1cc(-c2onc3ccc(-c4nnn[nH]4)cc23)cc(C2CCN(C3CCC3)CC2)c1] | ++ | | | | ¹H NMR (400 MHz, DMSO-d₆) δ 8.86 (s, 1H), 8.14-8.00 (m, 3H), 7.91 (dd, J = 9.4, 0.7 Hz, 1H), 7.68 (t, J = 7.8 Hz, 1H), 7.52 (d, J = 7.8 Hz, 1H), 3.65 (dd, J = 16.5, 8.4 Hz, 1H), 3.46 (d, J = 12.1 Hz, 2H), 3.12-3.01 (m, 2H), 2.93-2.84 (m, 2H), 2.35-2.17 (m, 4H), 2.16-2.00 (m, 3H), 1.83-1.67 (m, 2H); ¹³C NMR (101 MHz, DMSO-d₆) δ 178.55, 157.47, 146.41, 145.16, 130.62, 130.16, |
| | | | | | | 129.89, 127.65, 125.68, |
| | | | | | | 125.22, 121.57, 121.41, |
| | | | | | | 117.03, 113.99, 58.63, |
| | | | | | | 49.29, 38.91, 29.85, |
| | | | | | | 25.55, 13.59; MS (ESI) |
| | | | | | | for [M + H]⁺ |
| | | | | | | (C₂₃H₂₅N₆O⁺): calcd. |
| | | | | | | m/z 401.21; found m/z |
| | | | | | | 401.50. |
| 106 | 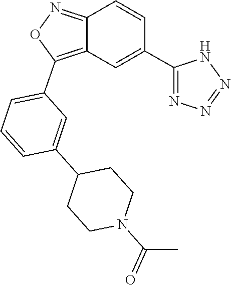 [SMILES: CC(=O)N1CCC(c2cccc(-c3onc4ccc(-c5nnn[nH]5)cc34)c2)CC1] | +++ | | | | ¹H NMR (400 MHz, CD₃OD + CDCl₃) δ 8.69 (s, 1H), 8.04 (dd, J = 9.4, 1.4 Hz, 1H), 7.99- 7.92 (m, 2H), 7.80 (dd, J = 9.4, 0.8 Hz, 1H), 7.59 (t, J = 7.7 Hz, 1H), 7.49 (d, J = 7.8 Hz, 1H), 4.60 (d, J = 13.1 Hz, 1H), 3.21-3.13 (m, 1H), 3.13-3.11 (m, 1H), 2.99-2.90 (m, 1H), 2.66-2.58 (m, 1H), 2.04 (s, 3H), 1.89 (t, J = 13.6 Hz, 2H), 1.77-1.67 (m, 1H), 1.63-1.51 (m, 1H); ¹³C NMR (101 MHz, |
| | | | | | | CD₃OD + CDCl₃) δ |
| | | | | | | 168.60, 166.89, 166.54, |
| | | | | | | 163.07, 157.38, 147.62, |
| | | | | | | 130.11, 129.98, 129.87, |
| | | | | | | 127.61, 125.53, 125.10, |
| | | | | | | 121.08, 116.68, 114.00, |
| | | | | | | 46.69, 42.22, 41.78, |
| | | | | | | 33.46, 32.85, 21.49; MS |
| | | | | | | (ESI) for [M + H]⁺ |
| | | | | | | (C₂₁H₂₁N₆O₂⁺): calcd. |
| | | | | | | m/z 389.17; found m/z |
| | | | | | | 389.45. |
| 107 | 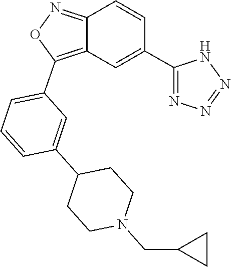 [SMILES: c1cc(-c2onc3ccc(-c4nnn[nH]4)cc23)cc(C2CCN(CC3CC3)CC2)c1] | +++ | | | | ¹H NMR (400 MHz, CD₃OD) δ 8.76 (d, J = 6.1 Hz, 1H), 8.08-7.98 (m, 3H), 7.78 (t, J = 7.9 Hz, 1H), 7.64 (t, J = 8.0 Hz, 1H), 7.54 (d, J = 7.6 Hz, 1H), 3.83 (d, J = 12.4 Hz, 2H), 3.26- 3.05 (m, 5H), 2.35- 2.07 (m, 4H), 1.26- 1.09 (m, 1H), 0.85- 0.74 (m, 2H), 0.48 (d, J = 4.8 Hz, 2H); ¹³C NMR (101 MHz, CD₃OD) δ 166.49, 157.32, 145.35, 129.83, 129.29, 129.19, |
| | | | | | | 129.15, 127.80, 125.15, |
| | | | | | | 124.89, 124.84, 120.78, |
| | | | | | | 116.18, 113.77, 61.68, |
| | | | | | | 52.49, 39.33, 30.19, |
| | | | | | | 5.13, 3.56; MS (ESI) for |
| | | | | | | [M + H]⁺ (C₂₃H₂₅N₆O⁺): |
| | | | | | | calcd. m/z 401.21; |
| | | | | | | found m/z 401.50. |
| 108 | 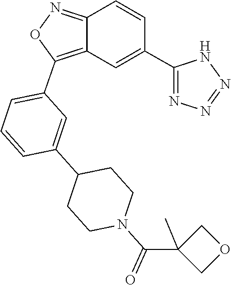 [SMILES: CC1(C(=O)N2CCC(c3cccc(-c4onc5ccc(-c6nnn[nH]6)cc45)c3)CC2)COC1] | ++ | | | | ¹H NMR (400 MHz, CDCl₃) δ 8.72 (s, 1H), 8.05 (d, J = 9.4 Hz, 1H), 7.96-7.82 (m, 2H), 7.71 (d, J = 9.4 Hz, 1H), 7.51 (t, J = 7.8 Hz, 1H), 7.34 (d, J = 7.9 Hz, 1H), 6.81 (s, 3H), 5.11 (dd, J = 14.9, 5.8 Hz, 2H), 4.76 (d, J = 12.2 Hz, 1H), 4.45 (dd, J = 9.8, 6.0 Hz, 2H), 3.32-3.03 (m, 2H), 2.99-2.84 (m, 1H), 2.76 (t, J = 12.2 Hz, 1H), 2.14-1.83 (m, 2H), 1.74 (s, J = 11.5 Hz, 4H), 1.43-1.18 (m, 1H); ¹³C NMR (101 |
| | | | | | | MHz, CDCl₃) δ 173.74, |
| | | | | | | 166.51, 157.39, 157.34, |
| | | | | | | 145.90, 129.88, 129.54, |
| | | | | | | 129.29, 127.95, 125.35, |
| | | | | | | 125.10, 121.65, 121.15, |
| | | | | | | 116.69, 114.21, 79.82, |
| | | | | | | 79.67, 46.18, 44.79, |
| | | | | | | 42.87, 42.15, 33.60, |
| | | | | | | 32.52, 23.38; MS (ESI) |
| | | | | | | for [M + H]⁺ |
| | | | | | | (C₂₅H₂₆N₆O₂⁺): calcd. |
| | | | | | | m/z 443.22; found m/z |
| | | | | | | 443.55. |
| 109 | 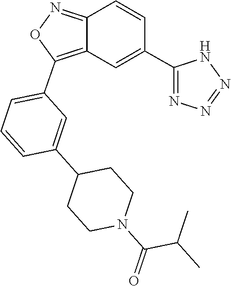 [SMILES: CC(C)C(=O)N1CCC(c2cccc(-c3onc4ccc(-c5nnn[nH]5)cc34)c2)CC1] | +++ | | | | ¹H NMR (400 MHz, CD₃OD + CDCl₃) δ 8.71 (s, 1H), 8.04-7.90 (m, 3H), 7.72 (d, J = 9.2 Hz, 1H), 7.56 (dd, J = 9.0, 6.3 Hz, 1H), 7.43 (d, J = 7.8 Hz, 1H), 4.74 (d, J = 14.7 Hz, 1H), 4.16 (d, J = 13.5 Hz, 1H), 3.22 (d, J = 11.7 Hz, 1H), 3.02- 2.86 (m, 2H), 2.78- 2.61 (m, 1H), 2.09- 1.93 (m, 2H), 1.78- 1.61 (m, 2H), 1.12 (dd, J = 15.6, 7.7 Hz, 6H); ¹³C NMR (101 MHz, CD₃OD + CDCl₃) δ 176.34, 166.82, 158.00, |
| | | | | | | 157.29, 146.72, 129.67, |
| | | | | | | 129.50, 129.42, 127.69, |
| | | | | | | 125.34, 125.01, 121.62, |
| | | | | | | 120.91, 116.25, 114.06, |
| | | | | | | 77.79, 77.47, 77.15, |
| | | | | | | 46.00, 42.52, 42.43, |
| | | | | | | 33.84, 32.69, 30.00, |
| | | | | | | 19.05, 18.78; MS (ESI) |
| | | | | | | for [M + H]⁺ |
| | | | | | | (C₂₃H₂₅N₆O₂⁺): calcd. |
| | | | | | | m/z 417.02; found m/z |
| | | | | | | 417.50. |
| 110 | 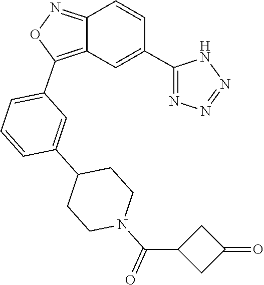 [SMILES: O=C1CC(C(=O)N2CCC(c3cccc(-c4onc5ccc(-c6nnn[nH]6)cc45)c3)CC2)C1] | +++ | | | | ¹H (400 MHz, CD₃OD + CDCl₃) δ 8.71 (s, 1H), 8.06-7.91 (m, 2H), 7.71 (d, J = 9.3 Hz, 1H), 7.56 (t, J = 7.8 Hz, 1H), 7.42 (d, J = 7.8 Hz, 1H), 4.75 (d, J = 13.6 Hz, 1H), 4.10 (d, J = 13.9 Hz, 1H), 3.59-3.34 (m, 3H), 3.31-3.20 (m, 5H), 2.99 (t, J = 12.3 Hz, 1H), 2.81 (t, J = 11.9 Hz, 1H), 2.11- 1.97 (m, 2H), 1.79- 1.64 (m, 2H); ¹³C NMR (101 MHz, CD₃OD + CDCl₃) δ 205.52, 171.51, 166.61, 158.11, |
| | | | | | | 157.37, 146.50, 129.70, |
| | | | | | | 129.64, 129.39, 127.80, |
| | | | | | | 125.34, 125.08, 122.28, |
| | | | | | | 120.55, 116.13, 114.17, |
| | | | | | | 50.62, 50.58, 46.06, |
| | | | | | | 42.90, 42.42, 33.56, |
| | | | | | | 32.61, 25.62. MS (ESI) |
| | | | | | | for [M + H]⁺ |
| | | | | | | (C₂₄H₂₃N₆O₃⁺): calcd. |
| | | | | | | m/z 443.18; found m/z |
| | | | | | | 443.50. |
| 111 | 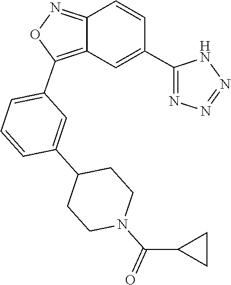 [SMILES: O=C(C1CC1)N1CCC(c2cccc(-c3onc4ccc(-c5nnn[nH]5)cc34)c2)CC1] | +++ | | | | ¹H NMR (400 MHz, CD₃OD + CDCl₃) δ 10.78 (s, 1H), 9.15 (s, 2H), 8.90 (s, 1H), 8.19 (d, J = 8.7 Hz, 2H), 8.06 (d, J = 9.3 Hz, 1H), 7.94 (d, J = 8.7 Hz, 2H), 7.85 (d, J = 9.4 Hz, 1H), 2.31 (dq, J = 14.0, 7.2 Hz, 1H), 2.10 (dq, J = 13.5, 7.2 Hz, 1H); ¹³C NMR (101 MHz, CD₃OD + CDCl₃) δ 171.11, 166.01, 157.05, 154.88, 141.71, 129.65, 127.78, 121.90, 121.61, 120.48, 119.97, 116.43, 113.11, 46.95, 45.06, 43.32, |
| | | | | | | 29.01; MS (ESI) for |
| | | | | | | [M + H]⁺ (C₁₉H₁₈N₇O₂⁺): |
| | | | | | | calcd. m/z 415.19; |
| | | | | | | found m/z 415.49. |
| 112 | 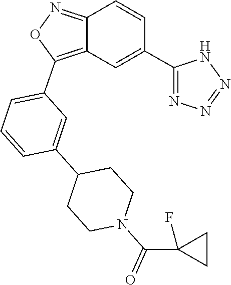 [SMILES: O=C(N1CCC(c2cccc(-c3onc4ccc(-c5nnn[nH]5)cc34)c2)CC1)C1(F)CC1] | +++ | | | | ¹H NMR (400 MHz, CD₃OD + CDCl₃) δ 8.72 (s, 1H), 8.05 (d, J = 9.0 Hz, 1H), 7.95-7.84 (m, 2H), 7.70 (d, J = 8.6 Hz, 1H), 7.50 (t, J = 7.5 Hz, 1H), 7.33 (d, J = 7.5 Hz, 1H), 4.59 (s, 2H), 3.24 (s, 1H), 3.02-2.70 (m, 2H), 1.86 (d, J = 91.9 Hz, 4H), 1.47-1.14 (m, 4H); ¹³C NMR (101 MHz, CD₃OD + CDCl₃) δ 166.85, 166.63, 157.38, 152.23, 146.54, 129.70, 129.46, 129.26, 127.75, 125.46, 125.12, 121.37, 121.09, 116.50, |
| | | | | | | 114.12, 79.03, 42.59; |
| | | | | | | MS (ESI) for [M + H]⁺ |
| | | | | | | (C₂₃H₂₂FN₆O₂⁺): calcd. |
| | | | | | | m/z 433.18; found m/z |
| | | | | | | 433.15. |
| 113 | 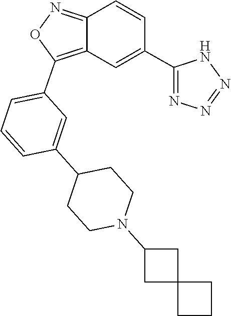 [SMILES: c1cc(-c2onc3ccc(-c4nnn[nH]4)cc23)cc(C2CCN(C3CC4(CCC4)C3)CC2)c1] | ++ | | | | ¹H NMR (400 MHz, CD₃OD + CDCl₃) δ 8.75 (s, 1H), 8.00 (d, J = 8.3 Hz, 3H), 7.73 (d, J = 6.4 Hz, 1H), 7.58 (s, 1H), 7.50 (s, 1H), 3.55 (s, 3H), 2.97 (s, 3H), 2.45 (s, 2H), 2.33-1.96 (m, 10H), 1.88 (s, 3H); ¹³C NMR (101 MHz, CD₃OD + CDCl₃) δ 166.69, 157.26, 152.61, 145.24, 129.87, 129.47, 129.22, 127.78, 125.36, 125.06, 121.11, 116.29, 116.24, 113.82, 55.72, 50.08, 37.64, 35.80, 34.01, 33.49, 29.90, 16.16; MS (ESI) for |
| | | | | | | [M + H]⁺ (C₂₆H₂₉N₆O⁺): |
| | | | | | | calcd. m/z 441.24; |
| | | | | | | found m/z 441.19. |
| 114 | 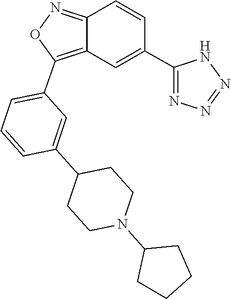 [SMILES: c1cc(-c2onc3ccc(-c4nnn[nH]4)cc23)cc(C2CCN(C3CCCC3)CC2)c1] | ++ | | | | ¹H NMR (400 MHz, CD₃OD + CDCl₃) δ 8.78 (s, 1H), 8.07-7.95 (m, 3H), 7.73 (d, J = 9.3 Hz, 1H), 7.57 (d, J = 7.6 Hz, 1H), 7.48 (d, J = 6.7 Hz, 1H), 3.71 (s, 2H), 3.49 (s, 2H), 3.13 (s, 4H), 2.25 (d, J = 39.9 Hz, 6H), 1.85 (d, J = 14.7 Hz, 4H), 1.68 (s, 2H); ¹³C NMR (101 MHz, CD₃OD + CDCl₃) δ 176.24, 155.82, 145.01, 129.93, 129.56, 129.18, 127.69, 125.58, 125.07, 121.22, 116.41, 113.81, 68.11, 52.29, 39.55, 30.27, 28.26, 23.50; MS |
| | | | | | | (ESI) for [M + H]⁺ |
| | | | | | | (C₂₄H₂₇N₇O⁺): calcd. |
| | | | | | | m/z 415.22; found m/z |
| | | | | | | 415.48. |
| 115 | 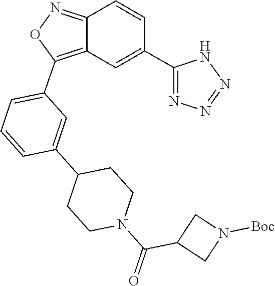 [SMILES: CC(C)(C)OC(=O)N1CC(C(=O)N2CCC(c3cccc(-c4onc5ccc(-c6nnn[nH]6)cc45)c3)CC2)C1] | ++ | | | | ¹H NMR (400 MHz, CD₃OD) δ 8.56 (s, J = 7.4 Hz, 1H), 7.93-7.84 (m, 3H), 7.66 (dd, J = 9.4, 0.8 Hz, 1H), 7.52 (t, J = 8.0 Hz, 1H), 7.45 (d, J = 7.9 Hz, 1H), 4.69 (d, J = 13.1 Hz, 1H), 4.19- 4.01 (m, 4H), 3.84- 3.67 (m, 2H), 3.30- 3.14 (m, 2H), 3.01- 2.90 (m, 1H), 2.86- 2.73 (m, 1H), 1.96 (dd, J = 13.6, 5.8 Hz, 2H), 1.75-1.59 (m, 2H), 1.42 (s, J = 4.5 Hz, 9H); ¹³C NMR (101 MHz, CD₃OD) δ 170.42, |
| | | | | | | 166.83, 157.24, 156.58, |
| | | | | | | 147.05, 129.56, 129.47, |
| | | | | | | 129.07, 127.53, 125.13, |
| | | | | | | 124.76, 120.89, 116.11, |
| | | | | | | 113.70, 79.83, 48.41, |
| | | | | | | 45.52, 42.38, 42.23, |
| | | | | | | 33.24, 32.42, 30.62, |
| | | | | | | 27.18; MS (ESI) for |
| | | | | | | [M + H]⁺ (C₂₈H₃₂N₇O₄⁺): |
| | | | | | | calcd. m/z 530.25; |
| | | | | | | found m/z 530.63. |
| 116 | 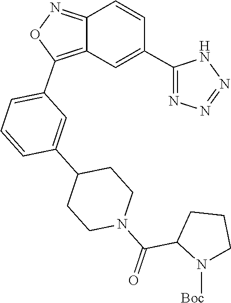 [SMILES: CC(C)(C)OC(=O)N1CCCC1C(=O)N1CCC(c2cccc(-c3onc4ccc(-c5nnn[nH]5)cc34)c2)CC1] | ++ | | | | ¹H NMR (400 MHz, CD₃OD) δ 8.76-8.64 (m, 1H), 8.46 (dd, J = 45.0, 8.5 Hz, 1H), 7.98 (dd, J = 18.3, 9.9 Hz, 2H), 7.79-7.70 (m, 1H), 7.62-7.40 (m, 2H), 4.80-4.58 (m, 2H), 4.18 (d, J = 15.0 Hz, 1H), 3.58-3.17 (m, 5H), 3.12-2.71 (m, 2H), 2.13-1.65 (m, 7H), 1.54-1.31 (m, 9H); ¹³C NMR (101 MHz, CD₃OD) δ 171.31, 157.33, 150.72, 146.82, 139.43, 129.97, 129.44, 128.48, 124.76, 121.46, 120.91, 120.66, |
| | | | | | | 116.08, 113.52, 79.70, |
| | | | | | | 56.84, 42.40, 33.18, |
| | | | | | | 32.21, 27.52, 27.39, |
| | | | | | | 27.23, 27.12, 23.07; MS |
| | | | | | | (ESI) for [M + H]⁺ |
| | | | | | | (C₂₉H₃₄N₇O₄⁺): calcd. |
| | | | | | | m/z 544.27; found m/z |
| | | | | | | 544.65. |
| 117 | 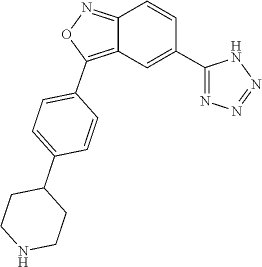 [SMILES: c1cc2noc(-c3ccc(C4CCNCC4)cc3)c2cc1-c1nnn[nH]1] | ++ | | | | ¹H NMR (400 MHz, CDCl₃ DMSO-d₆) δ 8.91 (d, J = 14.6 Hz, 2H), 8.77 (s, 1H), 8.18 (d, J = 8.2 Hz, 2H), 8.07 (d, J = 9.4 Hz, 1H), 7.87 (d, J = 9.3 Hz, 1H), 7.53 (d, J = 8.3 Hz, 2H), 3.67- 3.36 (m, 1H), 3.07- 2.94 (m, 4H), 2.03- 1.83 (m, 4H); ¹³C NMR (101 MHz, DMSO-d₆) δ 167.04, 155.54, 149.20, 130.67, 129.02, 128.23, 126.20, 122.48, 122.45, 117.46, 114.44, 110.52, 44.53, 39.89 |
| | | | | | | 30.09; MS (ESI) for |
| | | | | | | [M + H]⁺ (C₁₉H₁₉N₆O⁺): |
| | | | | | | calcd. m/z 347.16; |
| | | | | | | found m/z 347.40. |
| 118 | 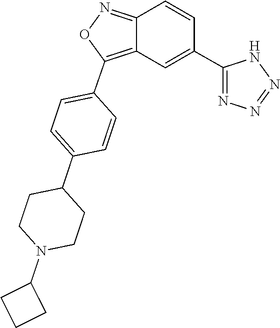 [SMILES: c1cc2noc(-c3ccc(C4CCN(C5CCC5)CC4)cc3)c2cc1-c1nnn[nH]1] | +++ | | | | ¹H NMR (400 MHz, CD₃OD) δ 8.74 (s, 1H), 8.11 (d, J = 8.3 Hz, 1H), 7.71 (d, J = 10.7 Hz, 2H), 7.55 (d, J = 8.2 Hz, 2H), 7.34 (d, J = 8.4 Hz, 1H), 3.68-3.46 (m, 4H), 2.97 (t, J = 15.4 Hz, 2H), 2.44-2.32 (m, 3H), 2.15 (d, J = 7.0 Hz, 3H), 1.98-1.77 (m, 3H), 0.84 (d, J = 7.2 Hz, 1H); ¹³C NMR (101 MHz, CD₃OD) δ 166.49, 157.32, 145.35, 129.83, 129.29, 129.19, 129.15, 127.80, 125.25, 124.89, 124.84, 120.78, 116.18, 113.77, 61.68, 52.49, 39.33, 30.19, |
| | | | | | | 5.13, 3.56; MS (ESI) for |
| | | | | | | [M + H]⁺ (C₂₃H₂₅N₆O⁺): |
| | | | | | | calcd. m/z 401.21; |
| | | | | | | found m/z 401.50. |
| 119 | 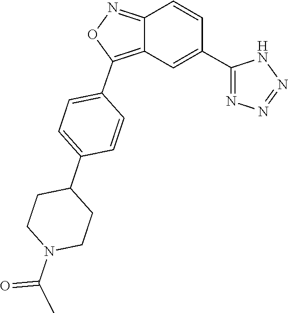 [SMILES: CC(=O)N1CCC(c2ccc(-c3onc4ccc(-c5nnn[nH]5)cc34)cc2)CC1] | ++ | | | | ¹H NMR (400 MHz, CD₃OD) δ 8.75 (s, 1H), 8.08 (d, J = 8.4 Hz, 2H), 8.03 (d, J = 9.3 Hz, 1H), 7.73 (d, J = 9.3 Hz, 1H), 7.64 (s, 1H), 7.50 (d, J = 8.3 Hz, 2H), 4.04 (d, J = 13.7 Hz, 1H), 3.25 (dd, J = 13.1, 2.4 Hz, 1H), 2.98-2.87 (m, 1H), 2.79-2.73 (m, 1H), 2.15 (s, 3H), 1.99 (dd, J = 23.1, 11.2 Hz, 2H), 1.79-1.62 (m, 2H), 0.89-0.79 (m, 1H); ¹³C NMR (101 MHz, CD₃OD) δ 170.00, 168.93, 161.58, 157.32, 148.67, 129.36, |
| | | | | | | 128.48, 127.85, 127.00, |
| | | | | | | 125.70, 120.84, 117.26, |
| | | | | | | 115.98, 46.82, 42.38, |
| | | | | | | 42.06, 33.20, 32.41, |
| | | | | | | 20.51. MS (ESI) for |
| | | | | | | [M + H]⁺ (C₂₁H₂₁N₆O₂⁺): |
| | | | | | | calcd. m/z 389.17; |
| | | | | | | found m/z 389.46. |
| 120 | 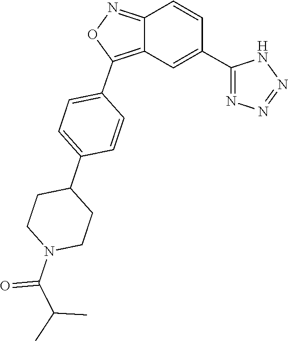 [SMILES: CC(C)C(=O)N1CCC(c2ccc(-c3onc4ccc(-c5nnn[nH]5)cc34)cc2)CC1] | ++ | | | | ¹H NMR (400 MHz CD₃OD) δ 8.74 (s, 1H), 8.07 (d, J = 8.4 Hz, 2H), 8.01 (dd, J = 9.4, 1.4 Hz, 1H), 7.72 (dd, J = 9.4, 0.9 Hz, 1H), 7.48 (d, J = 8.3 Hz, 2H), 4.76 (d, J = 13.3 Hz, 1H), 4.16 (d, J = 13.7 Hz, 1H), 3.24 (t, J = 12.1 Hz, 1H), 2.98-2.88 (m, 2H), 2.71 (dd, J = 12.9, 10.7 Hz, 1H), 2.00 (dd, J = 24.2, 14.3 Hz, 2H), 1.67 (qd, J = 12.7, 6.5 Hz, 2H), 1.14 (dd, J = 15.1, 6.7 Hz, 6H); ¹³C NMR (101 MHz, CD₃OD) δ 176.35, 166.72, 159.59, 157.32, 148.73, 129.36, 127.86, |
| | | | | | | 127.06, 125.77, 121.29, |
| | | | | | | 121.12, 116.22, 113.91, |
| | | | | | | 77.78, 77.46, 77.13, |
| | | | | | | 45.97, 42.58, 42.39, |
| | | | | | | 33.70, 30.02, 19.08, |
| | | | | | | 18.81 MS (ESI) for |
| | | | | | | [M + H]+ |
| | | | | | | (C23H25N6O2+): |
| | | | | | | calcd. m/z 417.20; |
| | | | | | | found m/z 417.50. |
| 121 | 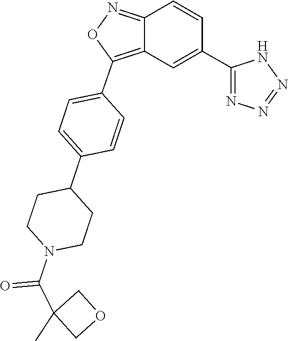 [SMILES: CC1(C(=O)N2CCC(c3ccc(-c4onc5ccc(-c6nnn[nH]6)cc45)cc3)CC2)COC1] | ++ | | | | ¹H NMR (400 MHz, CD₃OD) δ 8.75 (s, 1H), 8.09 (d, J = 8.4 Hz, 2H), 8.02 (dd, J = 9.4, 1.4 Hz, 1H), 7.74 (dd, J = 9.4, 0.9 Hz, 1H), 7.50 (d, J = 8.3 Hz, 2H), 5.00 (d, J = 5.2 Hz, 2H), 4.38 (t, J = 5.7 Hz, 2H), 3.27- 3.10 (m, 4H), 2.99- 2.70 (m, 3H), 2.00 (d, J = 12.0 Hz, 2H), 1.76- 1.63 (m, 5H); ¹³C NMR (101 MHz, CD₃OD) δ 173.56, 166.61, 161.24, 157.35, 148.41, 129.35, 127.84, 127.03, 125.68, 122.30, 121.45, 120.92, 116.20, 77.88, 77.56, 77.23, 44.72, 42.39, 42.25, 33.25, 32.21, |
| | | | | | | 22.77; MS (ESI) for |
| | | | | | | [M + H]⁺ |
| | | | | | | (C₂₅H₂₇N₆O2⁺): calcd. |
| | | | | | | m/z 443.22; found m/z |
| | | | | | | 443.55. |
| 122 | 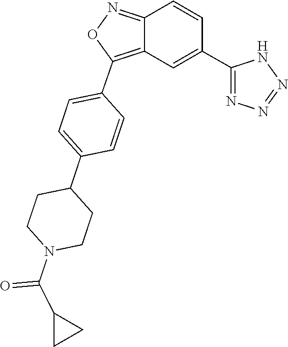 [SMILES: O=C(C1CC1)N1CCC(c2ccc(-c3onc4ccc(-c5nnn[nH]5)cc34)cc2)CC1] | ++ | | | | ¹H NMR (400 MHz, CD₃OD δ 8.75 (s, 1H), 8.08 (d, J = 8.3 Hz, 2H), 8.04-7.99 (m, 1H), 7.73 (d, J = 10.0 Hz, 1H), 7.50 (d, J = 8.3 Hz, 2H), 4.46 (d, J = 13.1 Hz, 1H), 2.96 (t, J = 12.2 Hz, 1H), 2.77 (t, J = 12.3 Hz, 1H), 2.10- 1.87 (m, 4H), 1.84- 1.59 (m, 3H), 0.94 (s, 2H), 0.83 (d, J = 8.1 Hz, 2H); ¹³C NMR (101 MHz, CD₃OD) δ 172.82, 166.73, 159.09, 157.23, 148.84, 129.36, 127.86, 127.01, 125.72, 121.27, 120.93, 116.16, 113.86, 46.05, 42.62, 33.47, 10.76, 6.98; MS |
| | | | | | | (ESI) for [M + H]⁺ |
| | | | | | | (C₂₃H₂₃N₆O2⁺): calcd. |
| | | | | | | m/z 415.19; found m/z |
| | | | | | | 415.50. |
| 123 | 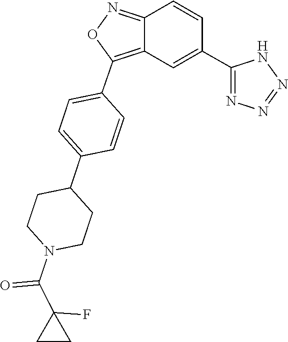 [SMILES: O=C(N1CCC(c2ccc(-c3onc4ccc(-c5nnn[nH]5)cc34)cc2)CC1)C1(F)CC1] | ++ | | | | ¹H NMR (400 MHz, CD₃OD) δ 8.74 (s, 1H), 8.08 (d, J = 8.4 Hz, 2H), 8.03 (dd, J = 9.4, 1.3 Hz, 1H), 7.73 (dd, J = 9.4, 0.8 Hz, 1H), 7.51 (d, J = 8.3 Hz, 2H), 4.58 (d, J = 9.4 Hz, 3H), 3.05- 2.93 (m, 2H), 2.07- 1.97 (m, 2H), 1.85- 1.66 (m, 2H), 1.27 (d, J = 18.2 Hz, 4H); ¹³C NMR (101 MHz, CD₃OD) δ 166.70, 157.31, 156.83, 154.14, 148.74, 129.40, 127.86, 126.99, 125.72, 121.52, 120.87, 116.11, 113.86, 77.90, 77.58, 77.25, 42.50, 33.21, 31.88, 24.56, 11.06; MS (ESI) |
| | | | | | | for [M + H]⁺ |
| | | | | | | (C₂₃H₂₂FN₆O₂⁺): calcd. |
| | | | | | | m/z 433.18; found m/z |
| | | | | | | 433.48. |

### Example 5: 3-(3-Phenylbenzo[c]isoxazol-5-yl)-1, 2,4-oxadiazol-5(4H)-one

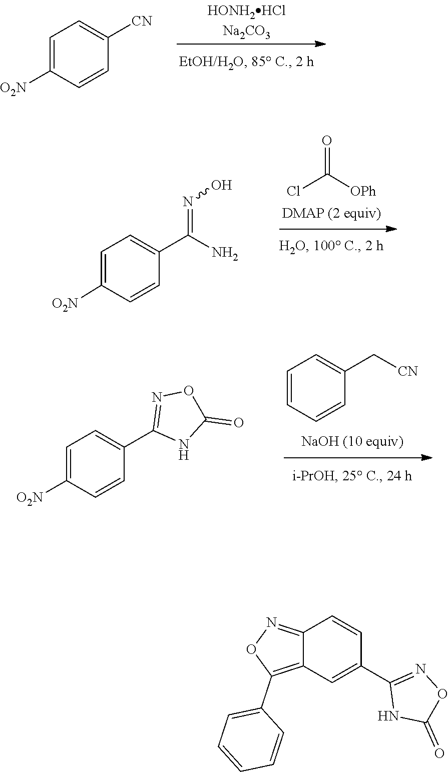

To a mixture of 4-nitrobenzonitrile (1.48 g, 10.0 mmol) in EtOH (30.0 mL) and water (6.0 mL) was added hydroxyamine hydrochloride (2.78 g, 40.0 mmol) and Na₂CO₃ (3.18 g, 40.0 mmol). The mixture was heated to 85° C. for 2 hr before it was cooled to room temperature. The solvent was evaporated under reduced pressure. The residue was then poured into brine. The mixture was extracted with EtOAc for three times. The combined organic phase was washed with brine and dried (Na₂SO₄). The solvent was then evaporated to afford N′-hydroxy-4-nitrobenzimidamide as an off-white solid (1.80 g, 9.94 mmol, 99%) without further purification. ¹H NMR (400 MHz, DMSO-d₆) δ 7.47-7.42 (m, 2H), 7.11-7.05 (m, 2H); MS (ESI) for [M+H]⁺ (C₇H₈N₃O₃⁺): calcd. m/z 182.06; found m/z 182.10; LC-MS: 97% purity; ¹H NMR 4:1 trans:cis.

To a mixture of N′-hydroxy-4-nitrobenzimidamide (1.80 g, 9.94 mmol) and DMAP (2.43 g, 19.9 mmol) in water (20 mL) was added phenyl chloroformate (1.87 mL, 14.9 mmol). The reaction mixture was refluxed at 100° C. for 2 hr before it was cooled to room temperature. To the mixture was added 1 M HCl to adjust pH to 3-4. The off-white precipitate was filtered and washed with water to afford 3-(4-nitrophenyl)-1,2,4-oxadiazol-5(4H)-one as an off-white solid (1.77 g, 8.54 mmol, 86%). ¹H NMR (400 MHz, DMSO-d₆) δ 13.20 (s, 1H), 8.43 (d, J=8.8 Hz, 2H), 8.07 (d, J=8.9 Hz, 2H); MS (ESI) for [M+H]⁺ (C₈H₆N₃O₄⁺): calcd. m/z 208.04; found m/z 208.00; LC-MS: 98% purity.

NaOH pellets were granulated (80.0 mg, 2.00 mmol) and added to 2-PrOH (2.0 mL). The mixture was stirred for 15 minutes before the addition of 3-(4-nitrophenyl)-1,2,4-oxadiazol-5(4H)-one (41.4 mg, 0.200 mmol). After the oxadiazolone was dissolved, benzyl cyanide (58 μL, 0.50 mmol) was added. The reaction mixture was stirred at room temperature and monitored by LCMS. Upon the completion of reaction, to the suspension was added HOAc to adjust pH to 5. The reaction mixture was poured into brine and extracted by EtOAc for three times. The combined organic phase was dried (Na₂SO₄) and the solvent was removed. The residue was purified by reverse phase chromatography to afford the 3-(3-phenylbenzo[c]isoxazol-5-yl)-1,2,4-oxadiazol-5(4H)-one as a yellow solid (50.6 mg, 0.181 mmol, 91%). ¹H NMR (400 MHz, DMSO-d₆) δ 8.57 (s, 1H), 8.21-8.10 (m, 2H), 7.87-7.74 (m, 2H), 7.75-7.62 (m, 3H). ¹³C NMR (101 MHz, DMSO-d₆) δ 166.42, 160.11, 157.04, 156.98, 131.62, 129.73, 127.21, 126.80, 126.70, 121.63, 120.16, 116.51, 113.15. MS (ESI) for [M+H]⁺ (C₁₅H₁₀N₃O₃⁺): calcd. m/z 280.07; found m/z 280.10.

Table 5 describes compounds prepared following procedures described in Example 5 using appropriate reagents. (Note: IC₅₀ (determined from enzyme-coupled assays): ++++ means <10 nM; +++ means between 10-100 nM, ++ means between 100 nM-1 μM; + means between 1-100 μM; − means inactive.)

| | | | | | | Physical Data |
|---|---|---|---|---|---|---|
| | | IP6K1 | IP6K2 | IP6K3 | IPMK | MS mz (M + 1) or |
| Cmpd | Structure | IC₅₀ | IC₅₀ | IC₅₀ | IC₅₀ | and ¹HNMR |
| 124 | 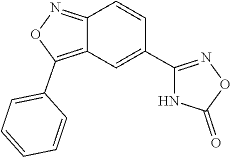 [SMILES: O=c1[nH]c(-c2ccc3noc(-c4ccccc4)c3c2)no1] | ++ | ++ | ++ | +++ | ¹H NMR (400 MHz, DMSO-d₆) δ 8.57 (s, 1H), 8.21-8.10 (m, 2H), 7.87-7.74 (m, 2H), 7.75-7.62 (m, 3H). ¹³C NMR (101 MHz, DMSO-d₆) δ 166.42, 160.11, 157.04, 156.98, 131.62, 129.73, |
| | | | | | | 127.21, 126.80, |
| | | | | | | 126.70, 121.63, |
| | | | | | | 120.16, 116.51, |
| | | | | | | 113.15. MS (ESI) for |
| | | | | | | [M + H]⁺ |
| | | | | | | (C₁₅H₁₀N₃O₃⁺): |
| | | | | | | calcd. m/z 280.07; |
| | | | | | | found m/z 280.10. |
| 125 | 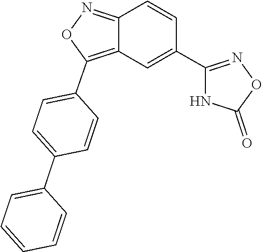 [SMILES: O=c1[nH]c(-c2ccc3noc(-c4ccc(-c5ccccc5)cc4)c3c2)no1] | +++ | +++ | ++++ | ++++ | ¹H NMR (400 MHz, DMSO-d₆) δ 8.63 (s, 1H), 8.25 (dd, J = 8.5, 2.1 Hz, 2H), 7.99 (dd, J = 8.5, 1.8 Hz, 2H), 7.89-7.78 (m, 4H), 7.55 (t, J = 7.6 Hz, 2H), 7.49- 7.43 (m, 1H). ¹³C NMR (101 MHz, DMSO-d₆) δ 166.46, 160.41, 157.40, 157.34, 143.32, 139.13, 129.61, 128.89, 128.18, 127.72, 127.57, |
| | | | | | | 127.33, 126.02, |
| | | | | | | 122.07, 120.42, |
| | | | | | | 116.92, 113.63. MS |
| | | | | | | (ESI) for [M + H]⁺ |
| | | | | | | (C₂₁H₁₄N₃O₃⁺): |
| | | | | | | calcd. m/z 356.10; |
| | | | | | | found m/z 356.10. |
| 126 | 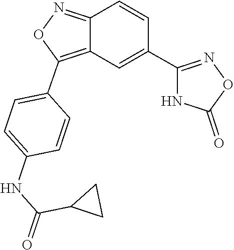 [SMILES: O=C(Nc1ccc(-c2onc3ccc(-c4noc(=O)[nH]4)cc23)cc1)C1CC1] | ++ | ++ | +++ | +++ | ¹H NMR (400 MHz, DMSO-d₆) δ 13.04 (br, 1H), 10.65 (s, 1H), 8.59 (d, J = 1.3 Hz, 1H), 8.13 (d, J = 8.2 Hz, 2H), 7.93 (d, J = 8.2 Hz, 2H), 7.86-7.73 (m, 2H), 1.90-1.80 (m, 1H), 0.98-0.73 (m, 4H). MS (ESI) for [M + H]⁺ (C₁₉H₁₅N₄O₄⁺): calcd. m/z 363.11; found m/z 363.10. |
| 127 | 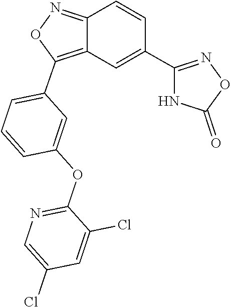 [SMILES: O=c1[nH]c(-c2ccc3noc(-c4cccc(Oc5ncc(Cl)cc5Cl)c4)c3c2)no1] | +++ | +++ | | +++ | ¹H NMR (400 MHz, DMSO-d₆) δ 8.56 (s, 1H), 8.39 (d, J = 2.3 Hz, 1H), 8.20 (d, J = 2.3 Hz, 1H), 8.09- 8.03 (m, 1H), 7.97 (t, J = 2.0 Hz, 1H), 7.87 (d, J = 9.4 Hz, 1H), 7.83-7.73 (m, 2H), 7.49 (dd, J = 8.2, 2.3 Hz, 1H); ¹³C NMR (101 MHz, DMSO- d₆) δ 165.43, 159.76, 157.01, 156.91, 156.81, 153.91, 143.75, 139.48, 131.29, 128.09, 127.28, 125.63, 124.60, 123.76, |
| | | | | | | 121.53, 120.24, |
| | | | | | | 119.55, 118.78, |
| | | | | | | 116.61, 113.40; MS |
| | | | | | | (ESI) for [M + H]⁺ |
| | | | | | | (C₂₀H₁₁Cl₂N₄O₄⁺): |
| | | | | | | calcd. m/z 441.02; |
| | | | | | | found m/z 441.00. |
| 128 | 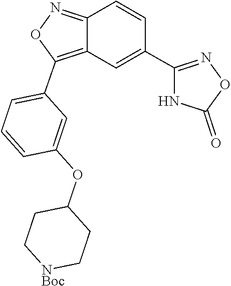 [SMILES: CC(C)(C)OC(=O)N1CCC(Oc2cccc(-c3onc4ccc(-c5noc(=O)[nH]5)cc34)c2)CC1] | +++ | +++ | | +++ | ¹H NMR (400 MHz, CD₃OD) δ 8.53 (t, J = 1.2 Hz, 1H), 7.90 (dd, J = 9.5, 1.3 Hz, 1H), 7.74 (ddd, J = 7.8, 1.7, 0.9 Hz, 1H), 7.69-7.61 (m, 2H), 7.56 (t, J = 8.0 Hz, 1H), 7.20 (ddd, J = 8.4, 2.6, 0.9 Hz, 1H), 4.79 (dt, J = 7.3, 3.8 Hz, 2H), 3.76 (ddd, J = 12.1, 7.4, 3.9 Hz, 2H), 3.41 (t, J = 10.5 Hz, 2H), 2.02 (ddd, J = 14.6, 7.3, 3.7 Hz, 2H), 1.82-1.70 (m, 2H), 1.48 (s, 9H); |
| | | | | | | MS (ESI) for |
| | | | | | | [M + H]⁺ |
| | | | | | | (C₂₅H₂₆N₄O₆Na⁺): |
| | | | | | | calcd. m/z 501.18; |
| | | | | | | found m/z 501.20. |
| 129 | 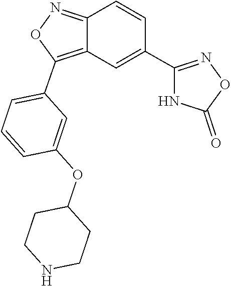 [SMILES: O=c1[nH]c(-c2ccc3noc(-c4cccc(OC5CCNCC5)c4)c3c2)no1] | +++ | +++ | | ++++ | ¹H NMR (400 MHz, DMSO-d₆) δ 9.11 (s, 1H), 8.70 (s, 1H), 7.87-7.79 (m, 3H), 7.74 (t, J = 2.1 Hz, 1H), 7.61 (t, J = 8.0 Hz, 1H), 7.32 (dd, J = 8.1, 2.4 Hz, 1H), 4.93 (dt, J = 7.9, 4.3 Hz, 1H), 3.09 (d, J = 8.9 Hz, 2H), 2.18 (dd, J = 15.6, 5.9 Hz, 2H), 2.00-1.88 (m, 2H) (one proton is overlapping with DMSO moisture); MS (ESI) for [M + H]⁺ |
| | | | | | | (C₂₀H₁₉N₄O₄⁺): |
| | | | | | | calcd. m/z 379.14; |
| | | | | | | found m/z 379.15. |
| 130 | 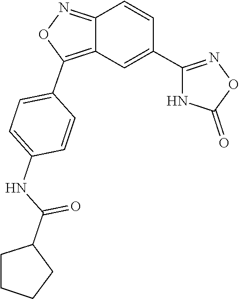 [SMILES: O=C(Nc1ccc(-c2onc3ccc(-c4noc(=O)[nH]4)cc23)cc1)C1CCCC1] | ++++ | ++++ | | +++ | ¹H NMR (500 MHz, DMSO-d₆) δ 10.30 (s, 1H), 8.56 (d, J = 1.3 Hz, 1H), 8.14- 8.08 (m, 2H), 7.97- 7.91 (m, 2H), 7.83- 7.74 (m, 2H), 2.84 (p, J = 8.0 Hz, 1H), 1.95-1.84 (m, 2H), 1.81-1.65 (m, 4H), 1.58 (pt, J = 7.5, 4.1 Hz, 2H); ¹³C NMR (126 MHz, DMSO- d₆) δ 175.09, 166.39, 159.74, 156.93, 156.79, 142.47, 127.60, 127.03, 122.02, 120.98, 119.43, 119.31, |
| | | | | | | 116.33, 112.46; MS |
| | | | | | | (ESI) for [M + H]⁺ |
| | | | | | | (C₂₁H₁₉N₄O₄⁺): |
| | | | | | | calcd. m/z 391.14; |
| | | | | | | found m/z 391.10. |
| 131 | 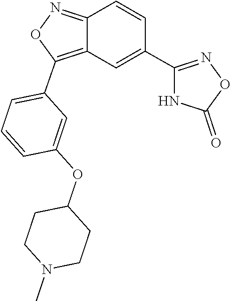 [SMILES: CN1CCC(Oc2cccc(-c3onc4ccc(-c5noc(=O)[nH]5)cc34)c2)CC1] | ++ | ++ | | ++ | ¹H NMR (400 MHz, DMSO-d₆) δ 8.67 (d, J = 52.1 Hz, 1H), 7.86-7.65 (m, 4H), 7.61 (t, J = 8.0 Hz, 1H), 7.34 (d, J = 7.8 Hz, 1H), 4.89 (d, J = 67.5 Hz, 1H), 3.15 (d, J = 12.5 Hz, 1H), 2.80 (d, J = 22.0 Hz, 3H), 2.35-1.89 (m, 4H) (three protons are overlapping with ¹³C NMR (101 MHz, DMSO-d₆) δ 166.23, 159.73, 157.04, 156.85, 131.16, 128.01, 127.29, 122.03, 120.06, |
| | | | | | | 119.91, 119.01, |
| | | | | | | 116.55, 115.14, |
| | | | | | | 114.09, 113.25, |
| | | | | | | 66.43, 48.46, 42.44, |
| | | | | | | 26.36; MS (ESI) for |
| | | | | | | [M + H]⁺ |
| | | | | | | (C₂₁H₂₁N₄O₄⁺): |
| | | | | | | calcd. m/z 393.16; |
| | | | | | | found m/z 393.20. |
| 132 | 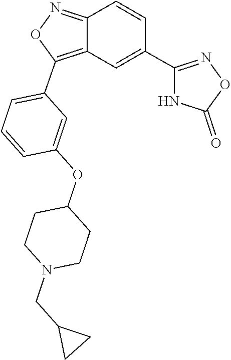 [SMILES: O=c1[nH]c(-c2ccc3noc(-c4cccc(OC5CCN(CC6CC6)CC5)c4)c3c2)no1] | ++ | ++ | | +++ | ¹H NMR (400 MHz, DMSO-d₆) δ 8.65 (s, 1H), 7.85-7.67 (m, 4H), 7.62 (t, J = 8.0 Hz, 1H), 7.33 (d, J = 8.3 Hz, 1H), 4.92 (s, 1H), 3.32-2.87 (m, 4H), 2.33-1.98 (m, 4H), 1.13 (p, J = 5.6 Hz, 1H), 0.65 (q, J = 5.6 Hz, 2H), 0.41 (q, J = 5.2 Hz, 2H); ¹³C NMR (101 MHz, DMSO-d₆) δ 166.19, 160.29, 157.33, 157.26, 157.08, 131.22, 128.07, 127.41, 121.82, 120.40, 120.35, 119.89, 118.93, 116.53, 113.31, 59.82, 5.46, 4.22 |
| | | | | | | (three sp² carbons |
| | | | | | | are missing); MS |
| | | | | | | (ESI) for [M + H]⁺ |
| | | | | | | (C₂₄H₂₅N₄O₄⁺): |
| | | | | | | calcd. m/z 433.19; |
| | | | | | | found m/z 433.20. |
| 133 | 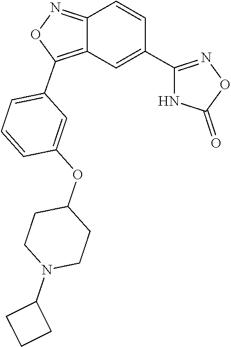 [SMILES: O=c1[nH]c(-c2ccc3noc(-c4cccc(OC5CCN(C6CCC6)CC5)c4)c3c2)no1] | ++ | ++ | | +++ | ¹H NMR (400 MHz, CD₃OD) δ 8.39 (s, 1H), 7.78-7.63 (m, 4H), 7.59 (t, J = 8.0 Hz, 1H), 7.28 (d, J = 8.4 Hz, 1H), 4.97 (s, 1H), 3.71 (d, J = 87.6 Hz, 2H), 3.47-2.94 (m, 5H), 2.53-2.08 (m, 8H), 2.04-1.82 (m, 3H); ¹³C NMR (101 MHz, CD₃OD) δ 167.87, 162.08, 158.70, 158.33, 132.23, 129.94, 128.48, 122.35, 121.77, 121.25, 119.85, 117.56, 115.78, 114.79, 68.13, 60.42, 45.79, 29.50, 27.79, 26.84, |
| | | | | | | 14.33 (one sp² |
| | | | | | | carbon is missing); |
| | | | | | | MS (ESI) for |
| | | | | | | [M + H]⁺ |
| | | | | | | (C₂₄H₂₅N₄O₄⁺): |
| | | | | | | calcd. m/z 433.19; |
| | | | | | | found m/z 433.20. |
| 134 | 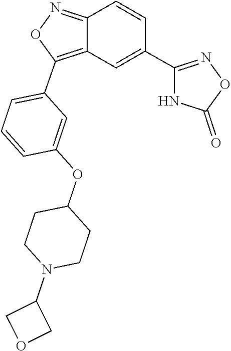 [SMILES: O=c1[nH]c(-c2ccc3noc(-c4cccc(OC5CCN(C6COC6)CC5)c4)c3c2)no1] | ++ | ++ | | ++ | ¹H NMR (400 MHz, DMSO-d₆) δ 8.65 (s, 1H), 7.87-7.70 (m, 4H), 7.62 (t, J = 8.0 Hz, 1H), 7.33 (d, J = 8.3 Hz, 1H), 4.95 (s, 1H), 4.84 (t, J = 7.4 Hz, 2H), 4.69 (t, J = 7.4 Hz, 2H), 4.42 (s, 1H), 3.34-2.92 (m, 4H), 2.31-2.14 (m, 2H), 2.05 (q, J = 7.4, 5.4 Hz, 2H); ¹³C NMR (101 MHz, DMSO-d₆) δ 166.24, 159.81, 157.32, 157.06, 156.91, 131.21, 128.04, 127.33, 121.94, 120.11, 119.89, 118.91, 116.59, 114.62, 113.28, |
| | | | | | | 71.93, 57.58, 48.67, |
| | | | | | | 46.11; MS (ESI) for |
| | | | | | | [M + H]⁺ |
| | | | | | | (C₂₃H₂₃N₄O₅⁺): |
| | | | | | | calcd. m/z 435.17; |
| | | | | | | found m/z 435.10. |
| 135 | 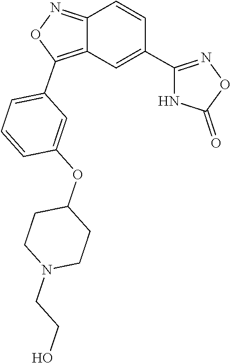 [SMILES: O=c1[nH]c(-c2ccc3noc(-c4cccc(OC5CCN(CCO)CC5)c4)c3c2)no1] | ++ | ++ | | +++ | ¹H NMR (400 MHz, DMSO-d₆) δ 8.69 (s, 1H), 7.92-7.67 (m, 4H), 7.63 (t, J = 8.0 Hz, 1H), 7.34 (dd, J = 8.3, 2.4 Hz, 1H), 5.36 (s, 1H), 4.92 (s, 1H), 3.79 (t, J = 5.2 Hz, 2H), 3.27-3.17 (m, 4H), 2.15 (d, J = 68.9 Hz, 4H) (Two protons overlap with DMSO moister peak); ¹³C NMR (101 MHz, DMSO- d₆) δ 166.20, 159.97, 157.04, 131.16, 128.02, 127.34 121.89, 120.22, 119.86, 118.91, 116.53, 113.27, 57.67, 55.10 (Three |
| | | | | | | sp² carbons and three |
| | | | | | | sp³ carbons are |
| | | | | | | missing); MS (ESI) |
| | | | | | | for [M + H]⁺ |
| | | | | | | (C₂₂H₂₃N₄O₅⁺): |
| | | | | | | calcd. m/z 423.17; |
| | | | | | | found m/z 423.15. |
| 136 | 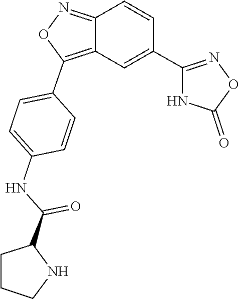 | ++ | ++ | | +++ | ¹H NMR (400 MHz, DMSO-d⁶) δ 13.25 (br, 1H), 11.35 (s, 1H), 9.83 (br, 1H), 8.77 (br, 1H), 8.69 (s, 1H), 8.21 (dd, J = 8.8, 1.9 Hz, 2H), 7.96 (d, J = 8.7 Hz, 2H), 7.77 (qd, J = 8.8, 8.2, 4.1 Hz, 2H), 4.53-4.37 (m, 1H), 3.30 (d, J = 7.8 Hz, 2H), 2.49-2.38 (m, 1H), 2.01 (dh, J = 25.4, 6.5 Hz, 3H); ¹³C NMR (101 MHz, DMSO-d⁶) δ 167.28, 165.87, 159.42, |
| | | | | | | 156.75, 156.55, |
| | | | | | | 140.87, 127.63, |
| | | | | | | 126.87, 121.98, |
| | | | | | | 121.81, 119.74, |
| | | | | | | 119.28, 116.22, |
| | | | | | | 112.44, 59.61. |
| | | | | | | 45.66, 29.53, 23.49; |
| | | | | | | [α]_{D}²⁰ = −28.8°, (c |
| | | | | | | 1.0, DMSO); MS |
| | | | | | | (ESI) for [M + H]⁺ |
| | | | | | | (C₂₀H₁₈N₅O₄⁺): |
| | | | | | | calcd. m/z 392.14; |
| | | | | | | found m/z 392.10. |
| 137 | 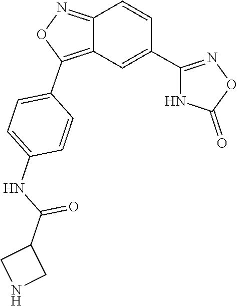 [SMILES: O=C(Nc1ccc(-c2onc3ccc(-c4noc(=O)[nH]4)cc23)cc1)C1CNC1] | ++ | + | | ++ | ¹H NMR (850 MHz, DMSO-d₆) δ 10.87 (s, 1H), 9.26 (br, 1H), 8.95 (br, 1H), 8.67 (s, 1H), 8.16 (d, J = 8.5 Hz, 2H), 7.94 (d, J = 8.3 Hz, 2H), 7.75 (q, J = 9.2 Hz, 2H), 4.13 (dt, J = 32.3, 10.1 Hz, 4H), 3.94-3.88 (m, 1H); ¹³C NMR (214 MHz, DMSO-d⁶) δ 169.27, 166.17, 159.77, 156.94, 156.86, 141.64, 127.71, 127.04, 122.15, 121.61, 119.73, 119.47, 116.30, |
| | | | | | | 112.55; MS (ESI) for |
| | | | | | | [M + H]⁺ |
| | | | | | | (C₁₉H₁₆N₅O₄⁺): |
| | | | | | | calcd. m/z 378.12; |
| | | | | | | found m/z 378.10. |
| 138 | 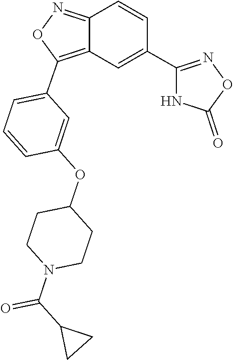 [SMILES: O=C(C1CC1)N1CCC(Oc2cccc(-c3onc4ccc(-c5noc(=O)[nH]5)cc34)c2)CC1] | ++ | +++ | | +++ | ¹H NMR (400 MHz, DMSO-d₆) δ 8.52 (s, 1H), 7.84 (d, J = 9.4 Hz, 1H), 7.78 (d, J = 10.2 Hz, 1H), 7.72 (dd, J = 7.7, 1.5 Hz, 1H), 7.68-7.58 (m, 2H), 7.32 (dd, J = 8.3, 2.5 Hz, 1H), 4.84 (tt, J = 7.9, 3.8 Hz, 1H), 4.12-3.86 (m, 2H), 3.62-3.36 (m, 2H), 2.14-1.90 (m, 3H), 1.62 (d, J = 40.5 Hz, 2H), 0.72 (tt, J = 7.9, 3.0 Hz, 4H); ¹³C NMR (101 MHz, DMSO-d₆) δ 171.06, 166.32, 159.80, 157.75, 157.01, 156.83, 131.13, 127.99, |
| | | | | | | 127.25, 121.71, |
| | | | | | | 120.01, 119.47, |
| | | | | | | 118.95, 116.58, |
| | | | | | | 114.25, 113.26, |
| | | | | | | 72.57, 42.14, 31.24, |
| | | | | | | 30.25, 10.39, 6.92; |
| | | | | | | MS (ESI) for |
| | | | | | | [M + H]⁺ |
| | | | | | | (C₂₄H₂₃N₄O₅⁺): |
| | | | | | | calcd. m/z 447.17; |
| | | | | | | found m/z 447.20. |
| 139 | 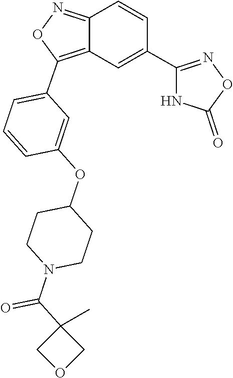 [SMILES: CC1(C(=O)N2CCC(Oc3cccc(-c4onc5ccc(-c6noc(=O)[nH]6)cc45)c3)CC2)COC1] | ++ | +++ | | ++ | ¹H NMR (400 MHz, DMSO-d₆) δ 8.52 (d, J = 1.3 Hz, 1H), 7.84 (d, J = 9.4 Hz, 1H), 7.78 (dd, J = 9.4, 1.4 Hz, 1H), 7.75-7.70 (m, 1H), 7.68-7.58 (m, 2H), 7.31 (dd, J = 8.2, 2.5 Hz, 1H), 4.82 (dd, J = 15.1, 4.9 Hz, 3H), 4.27 (d, J = 5.8 Hz, 2H), 3.90 (dd, J = 13.9, 6.3 Hz, 1H), 3.22-2.99 (m, 3H), 2.02 (d, J = 12.6 Hz, 2H), 1.72-1.59 (m, 2H), 1.55 (s, 3H); ¹³C NMR (101 MHz, DMSO-d₆) δ 172.23, 166.31, 159.75, 157.65, 157.01, 156.79, 131.13, 127.99, |
| | | | | | | 127.25, 121.72, |
| | | | | | | 119.99, 119.53, |
| | | | | | | 119.02, 116.59, |
| | | | | | | 114.24, 113.26, |
| | | | | | | 78.60, 78.47, 72.14, |
| | | | | | | 44.02, 42.05, 38.39, |
| | | | | | | 30.73, 30.02, 22.89; |
| | | | | | | MS (ESI) for |
| | | | | | | [M + H]⁺ |
| | | | | | | C₂₅H₂₅N₄O₆⁺): |
| | | | | | | calcd. m/z 477.18; |
| | | | | | | found m/z 477.20. |

### Example 6: 3-Phenylbenzo[d]isoxazole-5-carboxylic Acid

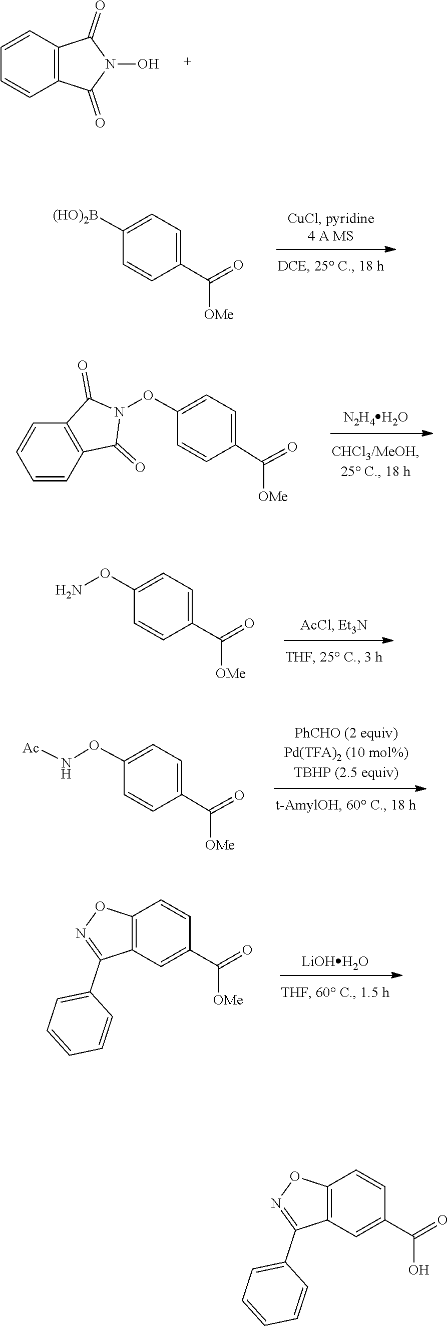

To a mixture of N-hydroxyphthalimide (200 mg, 1.23 mmol), (4-(methoxycarbonyl)phenyl)boronic acid (441 mg, 2.45 mmol), CuCl (121 mg, 1.23 mg) in DCE (6.0 mL) was added freshly activated 4 Å MS (308 mg) and pyridine (109 μL, 1.35 mmol). The reaction mixture was exposed to the air and was stirred at room temperature overnight. Upon completion, the precipitate was filtered and rinsed with CH₂Cl₂. The filtrate was concentrated under reduced pressure and the residue was purified by silica column chromatography (hexane/EtOAc gradient) to afford methyl 4-((1,3-dioxoisoindolin-2-yl)oxy)benzoate as a white solid (272 mg, 1.23 mmol, 75%). ¹H NMR (400 MHz, CDCl₃) δ 8.05 (dd, J=7.1, 2.1 Hz, 2H), 7.97-7.92 (m, 2H), 7.87-7.83 (m, 2H), 7.17 (dd, J=7.1, 2.1 Hz, 2H), 3.90 (s, 3H); MS (ESI) for [M+H]⁺ (C₁₆H₁₂NO₅⁺): calcd. m/z 298.07; found m/z 298.10; LC-MS: 96% purity.

To a solution of methyl 4-((1,3-dioxoisoindolin-2-yl)oxy)benzoate (267 mg, 0.897 mmol) in CHCl₃ (8.1 mL) and MeOH (0.9 mL) was added hydrazine monohydrate (218 μL, 4.49 mmol). The reaction was allowed to stir at room temperature overnight. The white precipitate was filtered and washed by CH₂Cl₂. The filtrate was concentrated and purified by silica column chromatography (hexane/EtOAc gradient) to afford methyl 4-(aminooxy)benzoate as a white solid (134 mg, 0.803 mmol, 90%). ¹H NMR (400 MHz, CDCl₃) δ 7.98 (d, J=9.0 Hz, 2H), 7.17 (d, J=9.0 Hz, 2H), 5.93 (s, 2H), 3.88 (s, 3H); MS (ESI) for [M+H]⁺ (C₈H₁₀NO₃⁺): calcd. m/z 168.07; found m/z 168.10; LC-MS: 98% purity.

To a solution of methyl 4-(aminooxy)benzoate (130 mg, 0.778 mmol) and Et₃N (217 μL, 1.56 mmol) in THF (3.7 mL) was added AcCl (166 μL, 2.34 mmol) at 0° C. The reaction was warmed up to room temperature and stirred for 3 hours. Upon the completion, the reaction mixture was quenched by MeOH and concentrated under reduced pressure. The residue was purified by reverse phase chromatography (MeCN/H₂O gradient) to afford methyl 4-(acetamidooxy)benzoate as a white solid (55.4 mg, 0.265 mmol, 34%) as a white solid. ¹H NMR (500 MHz, CDCl₃) δ 8.04-7.97 (m, 2H), 7.24-7.17 (m, 2H), 3.89 (d, J=0.5 Hz, 3H), 2.19 (d, J=0.5 Hz, 3H) (one NH proton is missing); MS (ESI) for [M+H]⁺ (C₁₀H₁₂NO₄⁺): calcd. m/z 210.08; found m/z 210.10; LC-MS: 95% purity.

To a solution of methyl 4-(acetamidooxy)benzoate (20.9 mg, 0.100 mmol) and Pd(TFA)₂ (3.32 mg, 10.0 μmol) in t-Amyl alcohol (0.4 mL) was added benzaldehyde (20.3 μL, 0.200 mmol) and TBHP (46 μL, 5.5 M in decane, 0.25 mmol). The reaction was degassed, charged with a nitrogen balloon, and heated up to 60° C. overnight. The reaction mixture was then cooled down to room temperature and poured into brine. The mixture was extracted by EtOAc. The combined organic phase was washed by 10% NaHSO₃ and brine and then dried (Na₂SO₄). The solvent was evaporated under reduced pressure. The residue was purified by silica column chromatography (hexane/EtOAc gradient) to afford methyl 3-phenylbenzo[d]isoxazole-5-carboxylate as an off-white solid (7.00 mg, 27.6 μmol, 28%). ¹H NMR (400 MHz, CDCl₃) δ 8.71-8.63 (m, 1H), 8.31 (dd, J=8.8, 1.7 Hz, 1H), 8.02-7.92 (m, 2H), 7.68 (d, J=8.9 Hz, 1H), 7.59 (dd, J=5.4, 2.0 Hz, 3H), 3.98 (s, 3H); MS (ESI) for [M+H]⁺ (C₁₅H₁₂NO₃⁺): calcd. m/z 254.08; found m/z 254.10; LC-MS: 95% purity.

To a solution of methyl 3-phenylbenzo[d]isoxazole-5-carboxylate (7.00 mg, 27.6 μmol) in THE (0.28 mL) was added LiOH H₂O (5.80 mg, 0.138 mmol). The reaction was heated up to 60° C. for 1.5 hours and acidified by TFA (21 μL, 0.28 mmol) after it was cooled down to room temperature. The solvent was removed under reduced pressure. The residue was purified by reverse phase chromatography (MeCN/H₂O gradient) to afford the title compound as a white solid (3.46 mg, 14.5 μmol, 52%). ¹H NMR (500 MHz, CD₃OD) δ 8.68 (s, 1H), 8.34 (d, J=8.8 Hz, 1H), 8.00 (d, J=6.8 Hz, 2H), 7.78 (d, J=8.8 Hz, 1H), 7.70-7.55 (m, 3H); ¹³C NMR (214 MHz, CD₃OD) δ 168.87, 167.28, 159.12, 132.75, 131.92, 130.48, 129.39, 129.15, 128.90, 126.21, 121.86, 111.07; MS (ESI) for [M+H]⁺ (C₁₄H₁₀NO₃⁺): calcd. m/z 240.07; found m/z 240.10; LC-MS: 96% purity.

Table 6 describes compounds prepared following procedures described in Example 6 using appropriate reagents. (Note: IC₅₀ (determined from enzyme-coupled assays): ++++ means <10 nM; +++ means between 10-100 nM, ++ means between 100 nM-1 μM; + means between 1-100 μM; − means inactive.)

| | | IP6K1 | IP6K2 | IP6K3 | IPMK | Physical Data |
|---|---|---|---|---|---|---|
| Cmpd | Structure | IC₅₀ | IC₅₀ | IC₅₀ | IC₅₀ | MS m/z (M + 1) or and ¹HNMR |
| 91 | 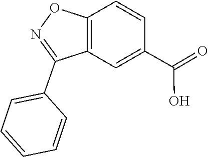 [SMILES: O=C(O)c1ccc2onc(-c3ccccc3)c2c1] | + | + | ++ | | ¹H NMR (500 MHz, CD₃OD) δ 8.68 (s, 1H), 8.34 (d, J = 8.8 Hz, 1H), 8.00 (d, J = 6.8 Hz, 2H), 7.78 (d, J = 8.8 Hz, 1H), 7.70- 7.55 (m, 3H); ¹³C NMR (214 MHz, CD₃OD) δ 168.87, 167.28, 159.12, 132.75, 131.92, 130.48, 129.39, 129.15, 128.90, 126.21, 121.86, 111.07; MS (ESI) for [M + H]⁺ |
| | | | | | | (C₁₄H₁₀NO₃⁺): calcd. m/z |
| | | | | | | 240.07; found m/z 240.10. |

### Example 7: Synthesis of 3-(4-nitrophenyl)-1H-1,2,4-triazole

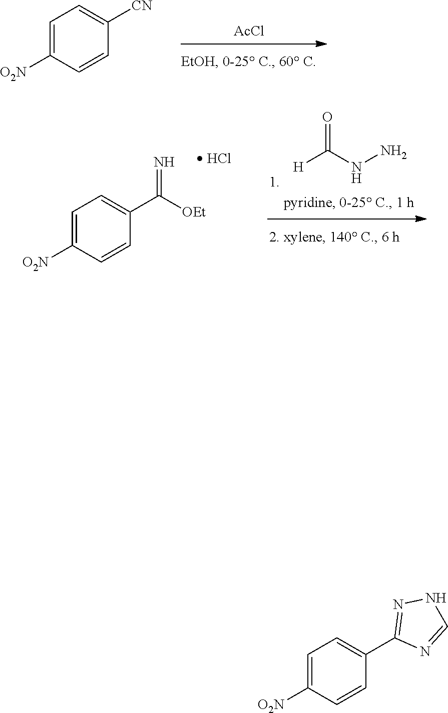

To a solution of 4-nitrobenzonitrile (296 mg, 2.00 mmol) in EtOH (1.5 mL) was added AcCl (1.14 mL, 16.0 mmol) in dropwise at 0° C. under nitrogen atmosphere. The reaction was warmed up to room temperature and stirred for 60 hours. The mixture became a light yellow slurry and was diluted with Et₂O. The resulting precipitate was filtered and washed with Et₂O to afford ethyl 4-nitrobenzimidate hydrochloride as an off-white solid (307 mg, 1.33 mmol, 66%). ¹H NMR (400 MHz, DMSO-d₆) δ 12.11 (s, 1H), 8.42 (d, J=8.4 Hz, 2H), 8.32 (d, J=8.5 Hz, 2H), 7.47-7.17 (m, 1H), 4.63 (q, J=6.8 Hz, 2H), 1.48 (t, J=6.8 Hz, 3H); MS (ESI) for [M+H]⁺ (C₉H₁₁N₂O₃⁺): calcd. m/z 195.08; found m/z 195.10; LC-MS: 98% purity.

To a solution of ethyl 4-nitrobenzimidate hydrochloride (295 mg, 1.28 mmol) in pyridine (1.3 mL) was added formohydrazide (92.0 mg, 1.53 mmol) at 0° C. The reaction was warmed to room temperature slowly over 1 hour. The yellow slurry was diluted with water. The resulting precipitate was filtered and washed with water to afford the formoimidohydrazide as a brown solid without further purification. The formoimidohydrazide was then dissolved in xylene (5.2 mL) and heated to 140° C. for 6 hours. The reaction was then cooled to room temperature and stirred overnight. The precipitate was filtered and washed with hexane and a few drops of methanol to afford the title compound as an off-white solid (128 mg, 0.671 mmol, 52%). ¹H NMR (400 MHz, DMSO-d₆) δ 8.67 (s, 1H), 8.34 (d, J=8.8 Hz, 2H), 8.27 (d, J=8.9 Hz, 2H); MS (ESI) for [M+H]+(C₈H₇N₄O₂⁺): calcd. m/z 191.06; found m/z 191.10; LC-MS: 99% purity.

### Example 8: Synthesis of 1-(4-nitrophenyl)-1H-1,2,4-triazole

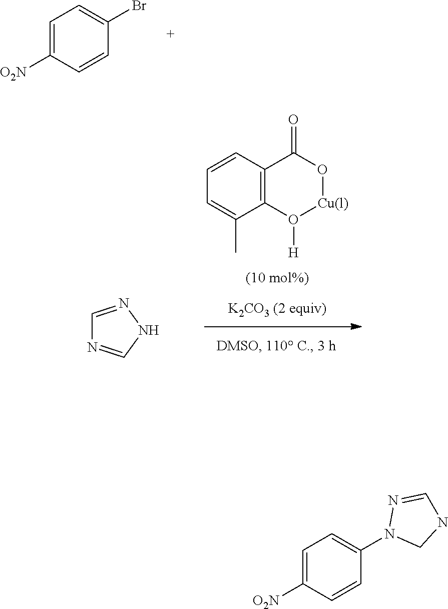

A mixture of 1-bromo-4-nitrobenzen (202 mg, 1.00 mmol), 1H-1,2,4-triazole (104 mg, 1.50 mmol), Cu(I)MeSal (21.5 mg, 0.100 mmol) and K₂CO₃ (276 mg, 2.00 mmol) in DMSO (5.0 mL) was degassed and charged with a nitrogen balloon. The reaction mixture was then heated up to 110° C. under nitrogen atmosphere for 3 hours. Then the reaction was cooled to room temperature and filtered. The filter cake was washed by DMSO and the filtrate was poured into ice. The mixture was extracted by EtOAc for three times. The combined organic phase was washed with brine twice and dried (Na₂SO₄). The solvent was evaporated under reduced pressure. The residue was purified by silica column chromatography to afford the title compound as a white solid (68.9 mg, 0.362 mmol, 36%). ¹H NMR (400 MHz, CDCl₃) δ 8.70 (s, 1H), 8.44-8.37 (m, 2H), 8.17 (s, 1H), 7.96-7.89 (m, 2H); MS (ESI) for [M+H]⁺ (C₈H₇N₄O₂⁺): calcd. m/z 191.06; found m/z 191.10; LC-MS: 96% purity.

### Example 9: Expression and Purification of Human Recombinant IP6Ks

Recombinant human IP6K2 and the human PPIP5K2 kinase domain were prepared as previously described.^{41, 44} Genscript synthesized the codon-optimized cDNAs for expression in Escherichia coli of 6×His-MBP tagged human IP6K1 (N-terminally tagged with 6×-His followed by MBP) and IP6K3 (N-terminally tagged with 6×-His followed by Sumo). The procedures for IP6K1 and IP6K2 expression and purification were identical to those used for IP6K2. The cells were disrupted using a constant cell disruption system (Constant Systems) under 20 kPsi. Recombinant IP6Ks were purified with a Ni-NTA agarose column (Qiagen) followed by a HiTrap Heparin HP column (GE Healthcare. As a final step, a Superdex 200 gel filtration column (GE Healthcare) was used with a running buffer of 150 mM NaCl and 20 mM Tris-HCl, pH 7.5. The purity of these proteins was estimated to be >80% as judged by SDS-PAGE. The purified proteins were stored in aliquots at −80° C.

### Example 10: Use of an Enzyme-Coupled Assay to Measure IP6K Activity

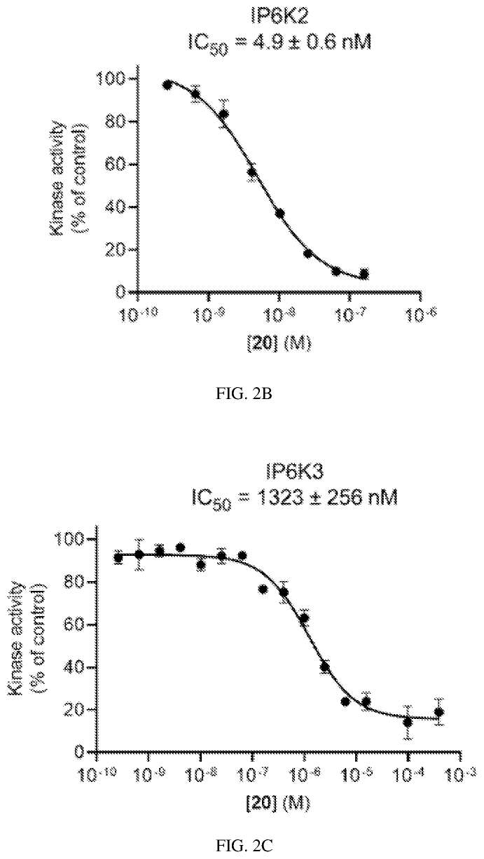

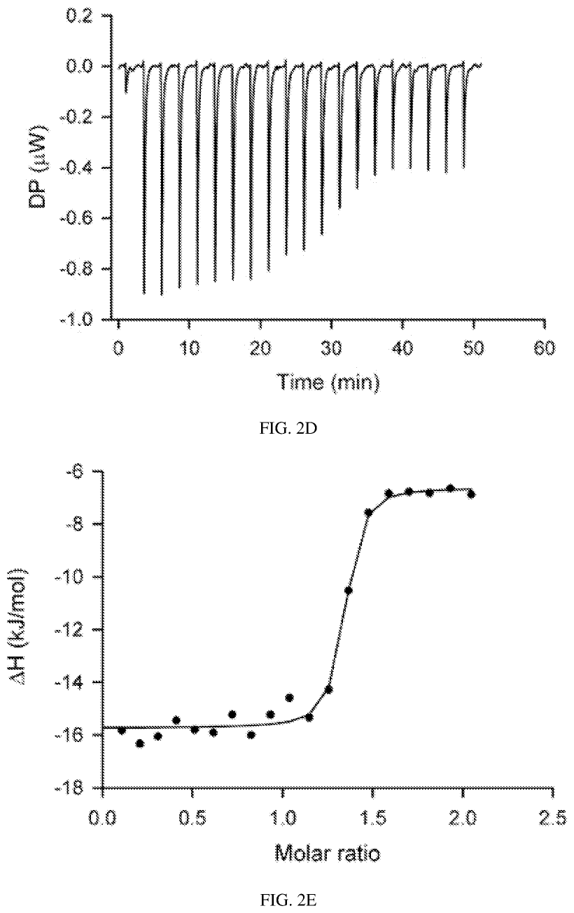

Enzyme activity was assayed at 37° C. in 50 μL reaction mixtures containing 100 nM IP6K1 or IP6K2, or 200 nM IP6K3, and unless otherwise indicated, 5.0 μM human Dipp1,⁴⁵ 20 mM HEPES (pH 7.2), 100 mM KCl, 3.5 mM MgCl₂, 20 μM EDTA, 25 μM InsP₆ and 500 μM ATP for 60-120 min. Pi release was determined with a malachite green colorimetric assay.⁴⁵ Where indicated, TNP (Cayman Chemical) was added to the assays (FIG. 2A-E).

### Example 9: HPLC Assays of Inositol Phosphate Kinase Activities

To assay IP6K activity, each reaction contained trace amounts (40,000 DPM) of ³H-InsP₆ (American Radiolabeled Chemicals, Inc., ART 1915) in a 100 μL incubations containing 20 mM HEPES (pH 7.2), 100 mM KCl, 3.5 mM MgCl₂, 20 μM EDTA, 1.0 mM ATP, 10 μM InsP₆, plus test compound in DMSO (or vehicle control) plus either 4.8 nM IP6K1, 3.5 nM IP6K2, or 8.9 nM IP6K3. To assay PPIP5K2 kinase activity, each reaction contained 12,500 dpm of 5-[³H]InsP₇⁴⁶ in 100 μL incubations containing 1.0 mM ATP, plus 40 nM PPIP5K2 kinase domain. Reactions were quenched after either 20 minutes (for PPIP5K2) or 180 minutes (for IP6Ks) by addition of 0.2 volumes of 2.0 M perchloric acid+1 mg/mL of InsP₆, and subsequently neutralized.

All assays were analyzed by HPLC, using a PartiSphere SAX 120 Å, 5 μm, 4.6×125 mm HPLC column. The elution gradient was generated by mixing Buffer A (1 mm Na₂EDTA) with Buffer B (Buffer A plus 2.5 M NH₄H₂PO₄, pH 3.9) and monitored by a Beta-RAM 6 in-line scintillation detector; 1.0 mL/min elute was mixed with 2.5 mL/min mono-flow scintillation liquid (FIG. 2A-2E).

### Example 11: Isothermal Titration Calorimetry

Calorimetry experiments were performed using a MicroCal PEAQ-ITC (Malvern Panalytical) with 40 μM recombinant MBP-tagged IP6K2 in the sample cell and 400 μM of inhibitor 20 in the syringe, each of which was maintained at 25° C. in buffer containing 20 mM Tris-HCl, pH 7.2, 150 mM KCl, 0.05 mM EDTA and 0.8 mM MgCl₂. The sample cell (volume=204 μL) and the syringe were cleaned before each run. Thermograms were constructed from 20 injections, each of which involved 2.0 μL of ligand delivered for 4.0 seconds, with an equilibration time of 150-300 seconds between each injection. The stirring speed was set to 750 rpm. Data were fitted to a single binding site model using the analysis software provided by the manufacturer. At least three runs were performed (FIG. 2A-2E).

### Example 12: Cell Culture and Assay of Intracellular Inositol Phosphates

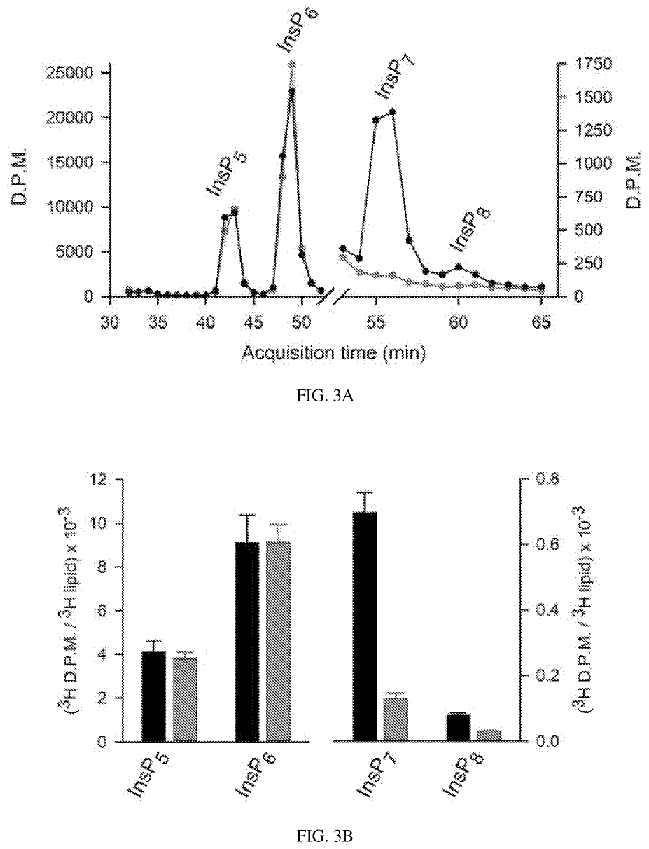

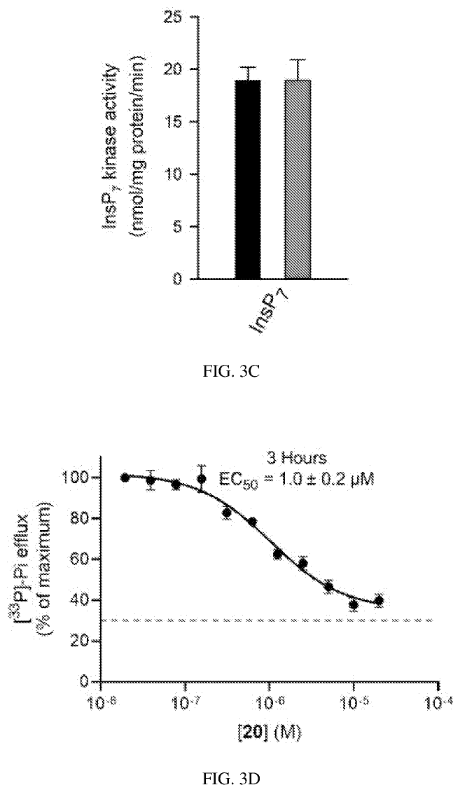

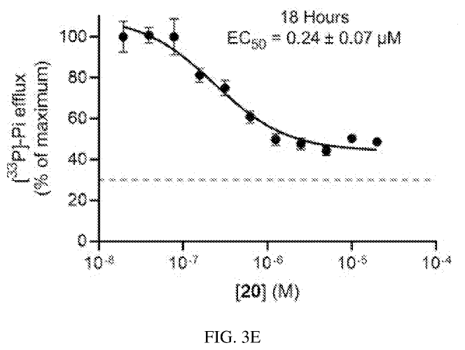

The HCT116 cells were cultured in DMEM/F-12 (Thermo Fisher Scientific) supplemented with 10% fetal bovine serum (FBS; Gemini Bio Products) and 100 units/mL penicillin-streptomycin (Thermo Fisher Scientific) at 37° C. in 95% O₂/5% CO₂. To assay inositol phosphates, 1×10⁶ cells were seeded in a 10-cm dish and cultured for 3 days in 7 mL of medium supplemented with 10 Ci/mL [³H]inositol (American Radiolabeled Chemicals) at which point cultures were 70% confluent. Cells were then incubated in medium with 2.5 μM inhibitor 20 or vehicle control for 3 hours. Cells were acid-quenched, and the inositol phosphates were extracted and resolved by HPLC using a 250×4.6 mm Synchropak Q100 HPLC column. Fractions of 1 minute were collected for 66 minutes, mixed with 2.5 mL of Monoflow-4 (National Diagonstics) and radiolabel was detected with a scintillation counter (FIG. 3A-3E).

### Example 13: Phosphate Efflux Assay

Phosphate efflux from cells was determined by slight modification of previously published procedures.⁴³ HCT116 cells were cultured in a 96-well plates for 1-2 days until 70% confluent. Cells were pre-treated with various concentration of inhibitor 20 for either 3 or 18 hours. The culture medium was then replaced with DMEM (Gibco catalog number: 11971-025) plus 10% FBS and 2.2×10⁵ dpm of [³³P]-Pi for 1 hour. Following this [³³P]-Pi labeling period, cells were washed at room temperature five times with 100 μL phosphate-buffered saline (PBS), then incubated for 2 hours at 37° C. with 100 μL pre-warmed, fresh DMEM in 95% O₂/5% CO₂ in order to assay [³³P]-Pi efflux. Throughout the [³³P]-Pi uptake and efflux protocols, fresh 20 was added at concentrations equivalent to those used during the pre-treatment period. Then the entire 100 μL medium was removed and added to scintillation fluid to assess [³³P]-Pi efflux. Additionally, the remaining cells were lysed with 100 μL PBS plus 1% triton-X100 and residual cell-associated ³³Pi was determined; [³³P]-Pi efflux is depicted as a percentage of total [³³P]-Pi (FIG. 3A-3E).

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

### References ADDIN EN.REFLIST

1. Montesi, L.; El Ghoch, M.; Brodosi, L.; Calugi, S.; Marchesini, G.; Dalle Grave, R., Long-term weight loss maintenance for obesity: a multidisciplinary approach. Diabetes, metabolic syndrome and obesity: targets and therapy 2016, 9, 37.
2. Srivastava, G.; Apovian, C., Future pharmacotherapy for obesity: new anti-obesity drugs on the horizon. Current obesity reports 2018, 7 (2), 147-161.
3. Williams, D. M.; Nawaz, A.; Evans, M., Drug therapy in obesity: a review of current and emerging treatments. Diabetes Therapy 2020, 11 (6), 1199-1216.
4. Kyrou, I.; Randeva, H. S.; Tsigos, C.; Kaltsas, G.; Weickert, M. O., Clinical Problems Caused by Obesity. In Endotext, Feingold, K. R.; Anawalt, B.; Boyce, A.; Chrousos, G.; de Herder, W. W.; Dhatariya, K.; Dungan, K.; Grossman, A.; Hershman, J. M.; Hofland, J.; Kalra, S.; Kaltsas, G.; Koch, C.; Kopp, P.; Korbonits, M.; Kovacs, C. S.; Kuohung, W.; Laferrere, B.; McGee, E. A.; McLachlan, R.; Morley, J. E.; New, M.; Purnell, J.; Sahay, R.; Singer, F.; Stratakis, C. A.; Trence, D. L.; Wilson, D. P., Eds. MDText.com, Inc. Copyright © 2000-2021, MDText.com, Inc.: South Dartmouth (MA), 2000.
5. Bliiher, M., Obesity: global epidemiology and pathogenesis. Nat Rev Endocrinol 2019, 15 (5), 288-298.
6. Ramasamy, A.; Laliberté, F.; Aktavoukian, S. A.; Lejeune, D.; DerSarkissian, M.; Cavanaugh, C.; Smolarz, B. G.; Ganguly, R.; Duh, M. S., Direct and Indirect Cost of Obesity Among the Privately Insured in the United States: A Focus on the Impact by Type of Industry. Journal of occupational and environmental medicine 2019, 61 (11), 877-886.
7. Wadden, T. A.; Berkowitz, R. I.; Sarwer, D. B.; Prus-Wisniewski, R.; Steinberg, C., Benefits of lifestyle modification in the pharmacologic treatment of obesity: a randomized trial. Archives of internal medicine 2001, 161 (2), 218-227.
8. Kim, G. W.; Lin, J. E.; Blomain, E. S.; Waldman, S. A., Antiobesity pharmacotherapy: new drugs and emerging targets. Clinical Pharmacology & Therapeutics 2014, 95 (1), 53-66.
9. Vilar-Gomez, E.; Martinez-Perez, Y.; Calzadilla-Bertot, L.; Torres-Gonzalez, A.; Gra-Oramas, B.; Gonzalez-Fabian, L.; Friedman, S. L.; Diago, M.; Romero-Gomez, M., Weight Loss Through Lifestyle Modification Significantly Reduces Features of Nonalcoholic Steatohepatitis. Gastroenterology 2015, 149 (2), 367-78.e5; quiz e14-5.
10. Wing, R. R.; Lang, W.; Wadden, T. A.; Safford, M.; Knowler, W. C.; Bertoni, A. G.; Hill, J. O.; Brancati, F. L.; Peters, A.; Wagenknecht, L., Benefits of modest weight loss in improving cardiovascular risk factors in overweight and obese individuals with type 2 diabetes. Diabetes Care 2011, 34 (7), 1481-6.
11. Zhu, Q.; Ghoshal, S.; Rodrigues, A.; Gao, S.; Asterian, A.; Kamenecka, T. M.; Barrow, J. C.; Chakraborty, A., Adipocyte-specific deletion of Ip6k1 reduces diet-induced obesity by enhancing AMPK-mediated thermogenesis. The Journal of clinical investigation 2016, 126 (11), 4273-4288.
12. Chakraborty, A.; Koldobskiy, M. A.; Bello, N. T.; Maxwell, M.; Potter, J. J.; Juluri, K. R.; Maag, D.; Kim, S.; Huang, A. S.; Dailey, M. J., Inositol pyrophosphates inhibit Akt signaling, thereby regulating insulin sensitivity and weight gain. Cell 2010, 143 (6), 897-910.
13. Boregowda, S. V.; Ghoshal, S.; Booker, C. N.; Krishnappa, V.; Chakraborty, A.; Phinney, D. G., IP6K1 reduces mesenchymal stem/stromal cell fitness and potentiates high fat diet-induced skeletal involution. Stem Cells 2017, 35 (8), 1973-1983.
14. Mukherjee, S.; Haubner, J.; Chakraborty, A., Targeting the Inositol Pyrophosphate Biosynthetic Enzymes in Metabolic Diseases. Molecules 2020, 25 (6), 1403.
15. Zhu, Q.; Ghoshal, S.; Tyagi, R.; Chakraborty, A., Global IP6K1 deletion enhances temperature modulated energy expenditure which reduces carbohydrate and fat induced weight gain. Molecular metabolism 2017, 6 (1), 73-85.
16. Moritoh, Y.; Oka, M.; Yasuhara, Y.; Hozumi, H.; Iwachidow, K.; Fuse, H.; Tozawa, R., Inositol hexakisphosphate kinase 3 regulates metabolism and lifespan in mice. Scientific reports 2016, 6 (1), 1-13.
17. Jadav, R. S.; Kumar, D.; Buwa, N.; Ganguli, S.; Thampatty, S. R.; Balasubramanian, N.; Bhandari, R., Deletion of inositol hexakisphosphate kinase 1 (IP6K1) reduces cell migration and invasion, conferring protection from aerodigestive tract carcinoma in mice. Cellular signalling 2016, 28(8), 1124-1136.
18. Ghosh, S.; Shukla, D.; Suman, K.; Lakshmi, B. J.; Manorama, R.; Kumar, S.; Bhandari, R., Inositol hexakisphosphate kinase 1 maintains hemostasis in mice by regulating platelet polyphosphate levels. Blood, The Journal of the American Society of Hematology 2013, 122 (8), 1478-1486.
19. Hou, Q.; Liu, F.; Chakraborty, A.; Jia, Y.; Prasad, A.; Yu, H.; Zhao, L.; Ye, K.; Snyder, S. H.; Xu, Y., Inhibition of IP6K1 suppresses neutrophil-mediated pulmonary damage in bacterial pneumonia. Science translational medicine 2018, 10 (435).
20. Chakraborty, A., The inositol pyrophosphate pathway in health and diseases. Biological Reviews 2018, 93 (2), 1203-1227.
21. Rao, F.; Xu, J.; Fu, C.; Cha, J. Y.; Gadalla, M. M.; Xu, R.; Barrow, J. C.; Snyder, S. H., Inositol pyrophosphates promote tumor growth and metastasis by antagonizing liver kinase B1. Proceedings of the National Academy of Sciences 2015, 112 (6), 1773-1778.
22. Mukherjee, S.; Chakraborty, M.; Ulmasov, B.; McCommis, K.; Zhang, J.; Carpenter, D.; Msengi, E. N.; Haubner, J.; Guo, C.; Pike, D. P.; Ghoshal, S.; Ford, D. A.; Neuschwander-Tetri, B. A.; Chakraborty, A., Pleiotropic actions of IP6K1 mediate hepatic metabolic dysfunction to promote nonalcoholic fatty liver disease and steatohepatitis. Molecular Metabolism 2021, 101364.
23. Shears, S. B., Intimate connections: Inositol pyrophosphates at the interface of metabolic regulation and cell signaling. Journal of cellular physiology 2018, 233 (3), 1897-1912.
24. Wilson, Miranda S. C.; Livermore, Thomas M.; Saiardi, A., Inositol pyrophosphates: between signalling and metabolism. Biochemical Journal 2013, 452 (3), 369-379.
25. Illies, C.; Gromada, J.; Fiume, R.; Leibiger, B.; Yu, J.; Juhl, K.; Yang, S.-N.; Barma, D. K.; Falck, J. R.; Saiardi, A., Requirement of inositol pyrophosphates for full exocytotic capacity in pancreatic β cells. Science 2007, 318 (5854), 1299-1302.
26. Zhang, X.; Li, N.; Zhang, J.; Zhang, Y.; Yang, X.; Luo, Y.; Zhang, B.; Xu, Z.; Zhu, Z.; Yang, X., 5-IP7 is a GPCR messenger mediating neural control of synaptotagmin-dependent insulin exocytosis and glucose homeostasis. Nature Metabolism 2021, 3 (10), 1400-1414.
27. Szijgyarto, Z.; Garedew, A.; Azevedo, C.; Saiardi, A., Influence of inositol pyrophosphates on cellular energy dynamics. Science 2011, 334 (6057), 802-805.
28. Rao, F.; Cha, J.; Xu, J.; Xu, R.; Vandiver, M. S.; Tyagi, R.; Tokhunts, R.; Koldobskiy, M. A.; Fu, C.; Barrow, R., Inositol pyrophosphates mediate the DNA-PK/ATM-p53 cell death pathway by regulating CK2 phosphorylation of Tti1/Tel2. Molecular cell 2014, 54 (1), 119-132.
29. Jadav, R. S.; Chanduri, M. V.; Sengupta, S.; Bhandari, R., Inositol pyrophosphate synthesis by inositol hexakisphosphate kinase 1 is required for homologous recombination repair. Journal of Biological Chemistry 2013, 288 (5), 3312-3321.
30. Gu, C.; Nguyen, H.-N.; Ganini, D.; Chen, Z.; Jessen, H. J.; Gu, Z.; Wang, H.; Shears, S. B., KO of 5-InsP7 kinase activity transforms the HCT116 colon cancer cell line into a hypermetabolic, growth-inhibited phenotype. Proceedings of the National Academy of Sciences 2017, 114 (45), 11968-11973.
31. Kröber, T.; Bartsch, S. M.; Fiedler, D., Pharmacological tools to investigate inositol polyphosphate kinases-Enzymes of increasing therapeutic relevance. Advances in biological regulation 2021, 100836.
32. Padmanabhan, U.; Dollins, D. E.; Fridy, P. C.; York, J. D.; Downes, C. P., Characterization of a Selective Inhibitor of Inositol Hexakisphosphate Kinases. Journal of Biological Chemistry 2009, 284 (16), 10571-10582.
33. Ghoshal, S.; Zhu, Q.; Asteian, A.; Lin, H.; Xu, H.; Ernst, G.; Barrow, J. C.; Xu, B.; Cameron, M. D.; Kamenecka, T. M.; Chakraborty, A., TNP [N2-(m-Trifluorobenzyl), N6-(p-nitrobenzyl) purine]ameliorates diet induced obesity and insulin resistance via inhibition of the IP6K1 pathway. Molecular metabolism 2016, 5 (10), 903-917.
34. Wormald, M. M.; Ernst, G.; Wei, H.; Barrow, J. C., Synthesis and characterization of novel isoform-selective IP6K1 inhibitors. Bioorganic & medicinal chemistry letters 2019, 29 (19), 126628.
35. Shears, S. B.; Wang, H., Inositol phosphate kinases: Expanding the biological significance of the universal core of the protein kinase fold. Advances in biological regulation 2019, 71, 118-127.
36. Knight, Z. A.; Shokat, K. M., Features of selective kinase inhibitors. Chemistry & biology 2005, 12 (6), 621-637.
37. Puhl-Rubio, A. C.; Stashko, M. A.; Wang, H.; Hardy, P. B.; Tyagi, V.; Li, B.; Wang, X.; Kireev, D.; Jessen, H. J.; Frye, S. V.; Shears, S. B.; Pearce, K. H., Use of Protein Kinase-Focused Compound Libraries for the Discovery of New Inositol Phosphate Kinase Inhibitors. SLAS DISCOVERY: Advancing Life Sciences R&D 2018, 23 (9), 982-988.
38. Liao, G.; Ye, W.; Heitmann, T.; Ernst, G.; DePasquale, M.; Xu, L.; Wormald, M.; Hu, X.; Ferrer, M.; Harmel, R. K., Identification of Small-Molecule Inhibitors of Human Inositol Hexakisphosphate Kinases by High-Throughput Screening. ACS Pharmacology & Translational Science 2021, 4 (2), 780-789.
39. Terao, Y.; Takahashi, M.; Hara, R.; Hidaka, K.; Furukawa, H.; Yamasaki, T.; Kasai, S. Preparation of 4-aryloxy-2′-oxo-1′,2′-dihydrospiro[cyclohexane-1,3′-indole]-5′-carboxylic acid and 4-aryloxy-2′-oxo-1′,2′-dihydrospiro[cyclohexane-1,3′-pyrrolo[3,2-b]pyridine]-5′-carboxylic acid derivatives as IP6K inhibitors. WO2018182051A1, 2018.
40. Moritoh, Y.; Abe, S.-i.; Akiyama, H.; Kobayashi, A.; Koyama, R.; Hara, R.; Kasai, S.; Watanabe, M., The enzymatic activity of inositol hexakisphosphate kinase controls circulating phosphate in mammals. Nature communications 2021, 12 (1), 1-15.
41. Wang, H.; DeRose, E. F.; London, R. E.; Shears, S. B., IP6K structure and the molecular determinants of catalytic specificity in an inositol phosphate kinase family. Nature communications 2014, 5, 4178.
42. Fujii, M.; York, J. D., A role for rat inositol polyphosphate kinases rIPK2 and rIPK1 in inositol pentakisphosphate and inositol hexakisphosphate production in rat-1 cells. Journal of Biological Chemistry 2005, 280 (2), 1156-1164.
43. Li, X.; Gu, C.; Hostachy, S.; Sahu, S.; Wittwer, C.; Jessen, H. J.; Fiedler, D.; Wang, H.; Shears, S. B., Control of XPR1-dependent cellular phosphate efflux by InsP8 is an exemplar for functionally-exclusive inositol pyrophosphate signaling. Proceedings of the National Academy of Sciences 2020, 117 (7), 3568-3574.
44. Wang, H.; Falck, J.; Hall, T. M. T.; Shears, S. B., Structural basis for an inositol pyrophosphate kinase surmounting phosphate crowding. Nature chemical biology 2012, 8 (1), 111-116.
45. Zong, G.; Jork, N.; Hostachy, S.; Fiedler, D.; Jessen, H. J.; Shears, S. B.; Wang, H., New structural insights reveal an expanded reaction cycle for inositol pyrophosphate hydrolysis by human DIPP1. The FASEB Journal 2021, 35 (2), e21275.
46. Weaver, J. D.; Wang, H.; Shears, S. B., The kinetic properties of a human PPIP5K reveal that its kinase activities are protected against the consequences of a deteriorating cellular bioenergetic environment. Bioscience reports 2013, 33 (2), e00022.
47. Ghoshal, S.; Zhu, Q.; Asteian, A.; Lin, H.; Xu, H.; Ernst, G.; Barrow, J. C.; Xu, B.; Cameron, M. D.; Kamenecka, T. M.; Chakraborty, A., TNP [N2-(m-Trifluorobenzyl), N6-(p-nitrobenzyl)purine]ameliorates diet induced obesity and insulin resistance via inhibition of the IP6K1 pathway. Molecular metabolism 2016, 5 (10), 903-17.

1. A compound of Formula II: 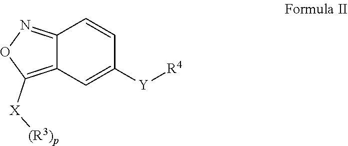
or a pharmaceutically acceptable salt thereof, wherein
X is aryl or heteroaryl;
R³ is, in each instance, selected from the group consisting of alkyl, cycloalkyl, heterocycloalkyl, aryl, heteroaryl, halo, -D, —OH, —OR⁵, —NH—COR⁶, —CONR⁷R⁸, —SO₂—NHR⁹, and NR¹⁰R¹¹
p is an integer from 0 to 5;
Y is present or absent, and when present selected from the group consisting of alkyl, cycloalkyl, and alkenyl; and
R⁴ is selected from the group consisting of —CO₂H, —CO₂R⁵, —CONR⁷R⁸, —SO₂—NHR⁶, —SO₃H, —P(O)(OH)(OR⁹), heteroaryl, and heterocycloalkyl; wherein
R⁵ is selected from alkyl, aryl, heteroaryl, and heterocycloalkyl; R⁶ is selected from alkyl, cycloalkyl, aryl, and heteroaryl; R⁷ and R⁸ are each independently selected from hydrogen, alkyl, cycloalkyl, heterocycloalkyl, aryl, and heteroaryl; R⁹ is selected from alkyl, aryl, heteroaryl, and heterocycloalkyl; and R¹⁰ and R¹¹ are each independently selected from hydrogen, alkyl, cycloalkyl, heterocycloalkyl, aryl, and heteroaryl.

2. The compound of claim 1, wherein Y is absent.

3. The compound of claim 2, wherein R⁴ is selected from the group consisting of —CO₂H, —CONR⁷R⁸, heteroaryl, and heterocycloalkyl.

4. The compound of claim 3, wherein R⁴ is selected from the group consisting of

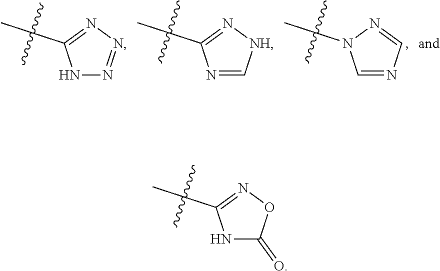 [SMILES: *c1nnn[nH]1 ; *c1nc[nH]n1 ; *n1cncn1 ; "," ; "and" ; *c1noc(=O)[nH]1]

5. (canceled)

6. (canceled)

7. (canceled)

8. The compound of claim 4, wherein X is aryl.

9. The compound of claim 1, wherein the compound is selected from a compound of Formula III, IV, and V:

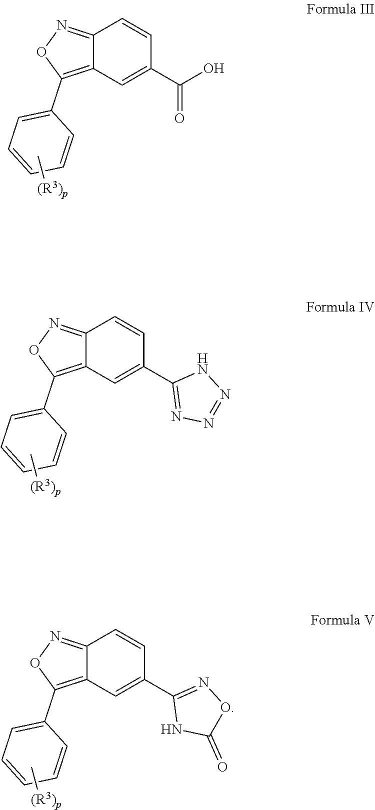

10. The compound of claim 9, wherein R³ is aryl or halo.

11. The compound of claim 9, wherein p is 1 and R³ is selected from —OR⁵, NH—COR⁶, alkyl, aryl, heteroaryl, and heterocycloalkyl, wherein R⁶ is alkyl, cycloalkyl or heterocycloalkyl.

12. (canceled)

13. The compound of claim 11, wherein the heterocycloalkyl is piperidinyl.

14. (canceled)

15. The compound of claim 11, wherein R⁵ is heteroaryl or heterocycloalkyl.

16. The compound of claim 15, wherein R⁵ is pyridinyl or piperdinyl.

17. (canceled)

18. (canceled)

19. A compound having one of the following structures:

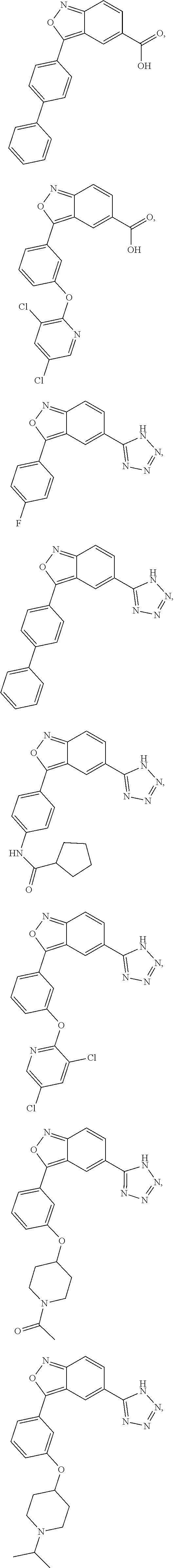

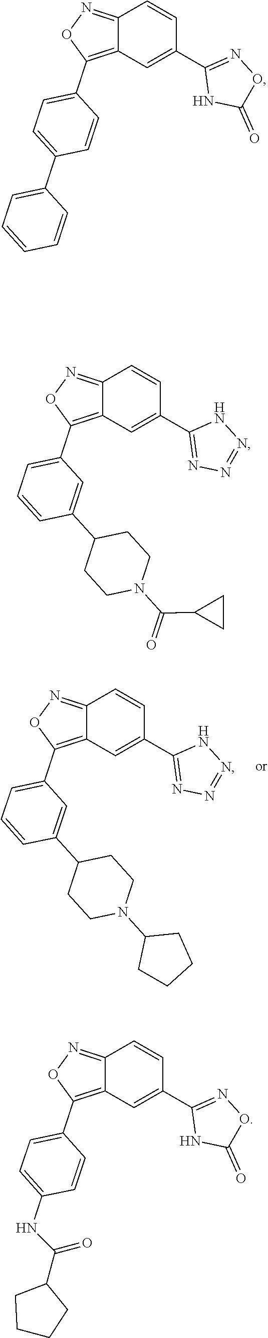 [SMILES: O=c1[nH]c(-c2ccc3noc(-c4ccc(-c5ccccc5)cc4)c3c2)no1 ; O=C(C1CC1)N1CCC(c2cccc(-c3onc4ccc(-c5nnn[nH]5)cc34)c2)CC1 ; c1cc(-c2onc3ccc(-c4nnn[nH]4)cc23)cc(C2CCN(C3CCCC3)CC2)c1 ; O=C(Nc1ccc(-c2onc3ccc(-c4noc(=O)[nH]4)cc23)cc1)C1CCCC1]

20. A pharmaceutical composition comprising a compound according to claim 1 or a pharmaceutically acceptable salt thereof, and one or more pharmaceutically acceptable carrier or excipient.

21. A method of preventing or treating a disease or disorder mediated by IP6K and/or IPMK, the method comprising administering a therapeutically effective amount of a compound of claim 1 to a subject in need thereof.

22. The method of claim 21, wherein the disease or disorder is selected from obesity, obesity related diseases, hyperphosphataemia, cancer, fungi infections, parasitic infections, and viral infections.

23. The method of claim 22, wherein the obesity related diseases are non-alcoholic fatty liver disease (NAFLD) or non-alcoholic steatohepatitis (NASH).

24. The method of claim 22, wherein the cancer is glioblastoma and the viral infection is corona virus.

25. (canceled)

26. The method of claim 22, wherein the disorder is hyperphosphataemia.

27. The method of claim 22, wherein the fungi infection is caused by a yeast strain selected from Cryptococcus neoformans and Candida albicans and the parasitic infection is caused by parasite Toxoplasma gondii.

28. (canceled)

29. (canceled)

30. A kit for treating a disease or disorder mediated by IP6K and/or IPMK, the kit comprising: 1) a pharmaceutical composition comprising a compound of Formula II from claim 1, and 2) instructions for use.

31. (canceled)
